(19) United States
(12) Patent Application Publication
(10) Pub. No.: US 2024/0317820 A1
(43) Pub. Date: Sep. 26, 2024

(54) IMPROVED BACILLUS PRODUCTION HOST

(71) Applicant: BASF SE, Ludwigshafen (DE)

(72) Inventors: Max Fabian Felle, Ludwigshafen (DE); Christopher Sauer, Ludwigshafen (DE); Mathis Appelbaum, Ludwigshafen (DE); Stefan Jenewein, Ludwigshafen (DE); Tobias Klein, Kaiserlautern (DE); Thomas Schweder, Greifswald (DE)

(21) Appl. No.: 18/572,974

(22) PCT Filed: Jun. 24, 2022
(86) PCT No.: PCT/EP2022/067440

§ 371 Date: Dec. 21, 2023

(30) Foreign Application Priority Data
Jun. 24, 2021 (EP) 21181351.4

(51) Int. Cl. C07K 14/32; C12N 9/54; C12N 15/75

(52) U.S. Cl. CPC C07K 14/32; C12N 9/54; C12N 15/75

(57) ABSTRACT

The present invention relates to a Bacillus host cell for increased production of biological compounds. Specifically, the invention relates to a Bacillus host with genetic modifications that lead to decreased production of components of biofilm and increased levels of phosphorylated De-gll. Said Bacillus host comprises at least one gene expression cassette with a polynucleotide encoding at least one polypeptide of interest, operably linked to a promoter for the production of compounds. The present invention further relates to a method for increased production of at least one polypeptide of interest based on cultivating the bacterial host cell of the present invention.

### FIELD OF THE INVENTION

The present invention relates to a Bacillus host cell for increased production of biological compounds. Specifically, the invention relates to a Bacillus host with genetic modifications that lead to decreased production of components of biofilm and increased levels of phosphorylated DegU. Said Bacillus host comprises at least one gene expression cassette with a polynucleotide encoding at least one polypeptide of interest, operably linked to a promoter for the production of compounds. The present invention further relates to a method for increased production of at least one polypeptide of interest based on cultivating the bacterial host cell of the present invention.

### BACKGROUND

Microorganisms of the Bacillus genus are widely applied as industrial workhorses for the production of valuable compounds, such as chemicals, polymers and proteins, in particular proteins like washing- and/or cleaning-active enzymes or enzymes used for feed and food applications. The biotechnological production of these useful substances is conducted via fermentation of such Bacillus species and subsequent purification of the product. Bacillus species are capable of secreting significant amounts of protein to the fermentation broth. This allows a simple product purification process compared to intracellular production and explains the success of Bacillus in industrial application.

Gram-positive microorganisms of the genus Bacillus have evolved cellular differentiation, adaptation and survival mechanisms to cope with changing environmental conditions such as heat, drought, water excess, nutrient limitations. Temporal and spatial distinct differentiation of Bacillus cells provides a survival strategy with individual cells fulfilling different functions like competence development and swarming motility, biofilm formation, production of extracellular degradative enzymes and synthesis antibiotics and spores. These differentiation processes are regulated via complex cell-to-cell signaling networks with integration via intracellular signal transduction cascades finally leading to differentiated gene expression.

Among different cellular regulator systems, the DegS-DegU two-component system controls various cellular processes in B. subtilis and related Bacillus species. In fact, the response regulator DegU and its cognate sensor kinase DegS control the expression of more than 100 genes (Mäder, U., Homuth, G.; 2002; Molecular Genetics and Genomics, 268(4), 455-467). Hereby the phosphorylation status of DegU as well as the overall level of phosphorylated DegU (DegU-P) within the cell determines differential gene expression (Verhamme, Daniel T.; Nicola R.; 2007; In: Molecular microbiology 65 (2), S. 554-568). Development of genetic competence requires unphosphorylated DegU, provided by basal expression of the degSU operon from the promoter located upstream of the operon. Expression of genes required for motility are activated by low levels of DegU-P, whereas biofilm formation is induced by medium levels of DegU-P with DegU-P controlling expression of bs/A, yvcA and the pga operon for synthesis poly-gamma-glutamic acid. Finally, high levels of DegU-P lead to enhanced exoenzyme production, while simultaneously repressing cellular processes activated by lower levels of DegU-P including biofilm formation (Msadek T, Dedonder R; 1990; J Bacteriol 172(2):824-834; Verhamme, Daniel T.; Nicola R.; 2007; In: Molecular microbiology 65 (2), S. 554-568). The required level of DegU-P for exoenzyme production is achieved by activation of a DegU-P positive feedback loop via the promoter immediately upstream of the degU gene.

In addition to DegS, the phosphorylation status of DegU is affected by DegQ, DegR, RapG, and PhrG. DegQ and DegR promote high levels of DegU-P by enhancing the DegS kinase activity and stabilizing DegU-P, respectively (Amory, A., Kunst, F., Aubert, E., Klier, A., & Rapoport, G; (1987); Journal of bacteriology, 169(1), 324-333.; Mukai, K.; Tanaka, T.; 1992; Journal of bacteriology 174 (24), S. 7954-7962.

In contrast, the response regulator aspartate phosphatase RapG controls DegU-P activity by interaction with the DNA binding domain of DegU-P thereby preventing DegU-P from binding to its target promoter which is countered by binding of PhrG to RapG (Ogura, M., Shimane, K., Asai, K., Ogasawara, N., & Tanaka, T.; 2003; In: Molecular microbiology, 49(6), 1685-1697.). Several mutations resulting in reduced genetic competence and enhanced exoenzyme secretion were mapped to genes encoding components of the DegS/DegU system. These so called “hy” (hyper-)mutations comprise but are not limited to the degU32 (DegU-H12L), degU31 (DegU-V131L), degS100 (DegS V236M), degS200 (DegS-G218E), degS-S76D (DegS-S76D), and degQ36 allele (Amory, A., Kunst, F., Aubert, E., Klier, A., & Rapoport, G; (1987); Journal of bacteriology, 169(1), 324-333.; Msadek T, Dedonder R; 1990; J Bacteriol 172(2):824-834). The degU32 allele encodes a more stable variant of DegU-P leading to higher levels of DegU-P and, consequently, enhanced exoenzyme expression (Msadek T, Dedonder R; 1990; J Bacteriol 172(2): 824-834). Similarly, the degS200 allele results in higher DegU-P levels due to reduced phosphatase activity of the DegS(hy) protein (Tanaka T, Kawata M, Mukai K; 1991; J Bacteriol. 173(17):5507-5515). Unlike degU32 and degS200 that result in amino acid changes within the respective proteins, the degQ36 mutation described for Bacillus subtilis encoding for a C->T mutation within the −10 box of the degQ promoter representing the actual wildtype degQ allele in undomesticated strains of Bacillus species (U.S. Pat. No. 5,264,350). As a consequence of domestication, in particular screening for high transformability (genetic competence), strains with reduced degQ expression evolved resulting in improved transformation efficiency but lower exoenzyme production. Thus, degQ36 represents the reverse mutation of the degQ allele found in domesticated strains (McLoon, Anna L; Losick, Richard; 2011; Journal of bacteriology 193 (8), S. 2027-2034.

In addition, overexpression of a degQ gene has been described in Bacillus subtilis to enhance extracellular enzyme production (U.S. Pat. Nos. 5,017,477, 5,264,350).

Biofilms of Bacillus species are characterized by the formation of a matrix exopolysaccharides (poly-N-acetyl glucosamine) and an amyloid-like protein TasA. The BslA protein (also referred to as hydrophobin), encoded by the yuaB gene, makes up the repellent surface layer, coats the biofilm containing colony and promotes complex colony morphology.

Biofilm formation in Bacillus requires expression of the operon tapA-sipW-tasA for production and processing of the TasA protein and expression of the epsA-O operon for synthesis and processing of the Eps (exopolysaccharides) (Branda, Steven S.; Kolter, Roberto; 2006; Molecular microbiology 59 (4), S. 1229-1238. The expression of tapA-sipW-tasA and epsA-O is complexly regulated, including control by the global regulator SinR, the transcriptional repressor AbrB, the anti-repressor proteins Sinl and SlrA and the regulator SIrR (formerly Slr) (Kobayashi, Kazuo, 2008; Molecular microbiology 69 (6), S. 1399-1410).

The global regulator SinR acts as a negative regulator repressing the expression of the two biofilm operons. The regulators Sinl and SlrA control SinR repression by binding to SinR and formation of inactive Sinl/SinR and SlrA/SinR heterocomplexes. But, the role of Sinl and SlrA in antagonizing SinR and the degree of overlapping functionality is poorly understood (Kobayashi, Kazuo; 2008; Molecular microbiology 69 (6), S. 1399-1410). SlrR acts as an activator of transcription of the two biofilm operons and the sirR expression is negatively regulated by SinR.

AbrB negatively regulates the transcription of the tapA-sipW-tasA operon. The expression of abrB and sinR are controlled by the abundance of phosphorylated SpoOA master regulator. The expression of the yuaB gene encoding for the BslA protein is regulated by the phosphorylated transcriptional activator DegU.

Only a limited number of studies have analyzed biofilm formation in the context of Bacillus strain optimization for increased production of compounds. Inactivation of sirR was shown to result in increased protease productivity in Bacillus subtilis (WO2003083125), however inactivation of the epsA-O operon (yveF-yvek; WO2003083125) did not result in increased protease productivity under identical cultivation conditions. Contrary to this observation deletion of the epsA-O operon in B. licheniformis strain 2709 resulted in 1.2-fold increased protease activity, with the aprE protease gene under control of the promoter PaprE (Zhou, C., Li, D.; Microb Cell Fact19,45 (2020). In contrast to enhanced protease expression upon deletion of epsA-O operon, overexpression of the epsE gene located within the epsA-O operon resulted in higher activity of the PaprE (Cairns, Lynne S.; Stanley-Wall, Nicola R.; 2013; Molecular microbiology 90 (1), S. 6-21). Inactivation of the tasA gene in Bacillus amyloliquefaciens reduced biofilm formation in pellicle formation, however, did not have a positive impact on amylase or levansucrase (SacB) productivity (Feng, Jun; Gao, Weixia; 2015; Scientific reports 5, S. 13814). Yet another study shows that inactivation of the epsA-O operon in Bacillus licheniformis results in increased transformation efficiency (WO 14/205096).

More interestingly, although TasA and Eps are major components of the Bacillus biofilm matrix, the transcription of tapA-sipW-tasA and epsA-O is not directly controlled by DegU-P. Instead, high levels of DegU-P promote biofilm formation by regulating transcription of the hydrophobic coat protein BslA, the lipoprotein YvcA and the genes involved in poly-gamma-glutamate (pga) production, namely ywsC (pgsB), ywtA (pgsC), ywtB (pgsA), ywtC (pgsE) (Stanley and Lazazzera; 2005; Molecular microbiology 57 (4), S. 1143-1158; Verhamme, Daniel T.; Nicola R.; 2007; In: Molecular microbiology 65 (2), S. 554-568)). On the contrary, high levels of DegU-P inhibit expression of tapA-sipW-tasA and epsA-O operons (Marlow, Victoria L.; Stanley-Wall, Nicola R.; 2014; Journal of bacteriology 196 (1), S. 16-27).

Hence, the role of biofilm formation and impact of the DegS-DegU two component regulatory systems on production of compounds remains elusive. For high-level production of compounds by recombinant production hosts, there is need for genetically modified Bacillus cells with reduced differentiation potential and optimized for recombinant production.

It remains the need for host cells allowing for enhanced production of a polypeptide of interest, preferably with an increased purity.

### BRIEF SUMMARY OF THE INVENTION

It has been found in the studies underlying the present invention that a Bacillus host cell with i) genetic modifications that lead to decreased production of components of biofilm, such as exopolymeric substances (EPS) and/or TasA and ii) genetic modifications that lead to increased levels of phosphorylated DegU allows for an efficient production of at least one polypeptide of interest, e.g. an exoenzyme, in said host cell.

Accordingly, the present invention relates to a modified Bacillus host cell comprising
i) a genetic modification which reduces the amount of exopolymeric substances (EPS) and/or a genetic modification which reduces the amount of the biofilm extracellular matrix component TasA, and
ii) a genetic modification which increases the amount of phosphorylated DegU as compared to a control cell.

In an embodiment, the genetic modification which reduces the amount of exopolymeric substances (EPS) is a genetic modification that reduces expression of the epsA-O operon.

In an embodiment, the genetic modification which reduces the amount of the biofilm extracellular matrix component TasA is a genetic modification that reduces expression of the tapA-sipW-tasA operon.

In a preferred embodiment, the genetic modification which reduces the amount of exopolymeric substances (EPS) and/or the genetic modification which reduces the amount of the biofilm extracellular matrix component TasA, is selected from
a) a genetic modification that inactivates the tapA-sipW-tasA operon,
b) a genetic modification that inactivates the epsA-O operon,
c) a genetic modification that inactivates the activator gene remA,
d) a genetic modification that inactivates the activator gene remB, and
e) a genetic modification that inactivates the sirA gene.

In a preferred embodiment, the genetic modification that inactivates the tapA-sipW-tasA operon is a deletion of tapA-sipW-tasA operon, or of a portion thereof.

In a preferred embodiment, the genetic modification that inactivates the epsA-O operon is a deletion of epsA-O operon, or of a portion thereof.

In a preferred embodiment, the genetic modification that inactivates the activator gene remA, is a deletion or missense mutation, preferably missense mutation, of the remA gene.

In a preferred embodiment, the genetic modification that inactivates the activator gene remB, is a deletion or missense mutation, preferably missense mutation, of the remB gene.

In another preferred embodiment, the both activator genes remA and remB are inactivated, preferably by missense mutation(s) of the remA and/or remB gene, preferably resulting in one or more non-conservative amino acid substitutions in the corresponding protein.

In a preferred embodiment, the genetic modification that inactivates the sirA gene is is a deletion of said gene, or of a portion thereof.

In a preferred embodiment of the modified host cell of the invention, the genetic modification which increases the amount of phosphorylated DegU is a genetic modification selected from
u1) a genetic modification causing increased expression of at least one of the genes selected from the group consisting of degU, degS, degQ and degR,
u2) a genetic modification causing decreased expression of the rapG gene or increased expression of the phrG gene,
u3) a genetic modification stabilizing the DegU phosphorylation state, such as a degU32 mutation,
u4) a genetic modification increasing autophosphorylation activity of the DegS protein, such as a DegS-S76D mutation, and/or
u5) a genetic modification reducing phosphatase activity of the DegS protein, such as a degS200 mutation.

In a preferred embodiment of the modified host cell of the invention, the genetic modification which increases the amount of phosphorylated DegU is a genetic modification is a degU32 mutation. Thus, the host cell comprises said mutation. Preferably, the degU32 gene is expressed under the control of the native promoters of the degU gene. Alternatively or additionally, the degQ gene may be overexpressed, e.g. by introducing and expressing an additional copy of degQ.

In a preferred embodiment of the modified host cell of the invention, the genetic modification which increases the amount of phosphorylated DegU is a mutation that causes increased expression of degQ, such as the introduction and expression of an additional copy of the degQ gene in the host cell.

In a preferred embodiment of the modified host cell of the invention, the host cell belongs to the species Bacillus pumilus, Bacillus velezensis, Bacillus amyloliquefaciens, Bacillus alcalophilus, Bacillus licheniformis, Bacillus paralicheniformis, Bacillus lentus, Bacillus clausii, Bacillus halodurans, Bacillus megaterium, Bacillus methanolicus, Geobacillus stearothermophilus (Bacillus stearothermophilus), Bacillus mojavensis, Bacillus globigii, or Bacillus subtilis. For example, the host cell belongs to the species. Bacillus pumilus, Bacillus velezensis, Bacillus amyloliquefaciens, Bacillus licheniformis or Bacillus subtilis.

In a preferred embodiment of the modified host cell of the invention, the host cell comprises an expression cassette for a polypeptide of interest. Preferably, the polypeptide of interest is an enzyme, such as an exoenzyme (i.e., an enzyme that is secreted by the cell into the fermentation broth). Preferably, the enzyme is a heterologous enzyme. For example, the enzyme selected from the group consisting of amylase, protease, lipase, phospholipase, mannanase, phytase, xylanase, phosphatase, glucoamylase, nuclease, galactosidase, endoglucanase and cellulase. The present invention further concerns a method for producing a polypeptide of interest, comprising
a) providing the modified Bacillus host cell of the present invention,
b) cultivating the host cell under conditions which allow for the expression of the polypeptide of interest, and
c) optionally isolating the polypeptide of interest from the cultivation medium.

In an embodiment of the method of the present invention, the cultivation in step b) is carried out as fedbatch cultivation.

### BRIEF DESCRIPTION OF THE DRAWINGS

FIG. 1: DegSU two component system. Model of the DegS-DegU two component regulatory system in Bacillus species. Depending on its degree of phosphorylation, DegU-P promotes motility, biofilm formation and exoenzyme production, while simultaneously repressing phenotypes controlled by lower levels of DegU-P. Unphosphorylated DegU is required for developing genetic competence. Increasing levels of DegU-P are indicated by the size of the grey circle including “P”. Phosphorylation of DegU is conducted by the sensor kinase DegS. Moreover, the phosphorylation status of DegU is affected by DegQ, DegR, RapG, and PhrG. DegQ and DegR promote high levels of DegU-P by enhancing the DegS kinase activity and stabilizing DegU-P, respectively. In contrast, the response regulator aspartate phosphatase RapG controls DegU-P activity by interaction with the DNA binding domain of DegU-P thereby preventing the degU-P from binding to its target promoter which is countered by binding of PhrG to RapG.

FIG. 2: Alignment of the promoter sequences within the 5′ region of the degQ gene from the indicated Bacillus species. The nucleotide position −61 to −119 relative to the translational start codon is depicted. The sequence numbering is relative to the promoter sequence of Bacillus subtilis. The −35 box and the −10 box of the core promoter region is shown. Within the −10 box the C-to T transition in domesticated Bacillus subtilis strain 168 is indicated.

FIG. 3: Protease productivity of different Bacillus licheniformis strains. The relative protease activity in percent is plotted against the indicated Bacillus licheniformis strains for the 72 h timepoints of fed-batch cultivation with the Bacillus licheniformis strain M409 set to 100% (grey/black checked design). The negative control strain Bacillus licheniformis strain M309 lacks the protease BLAP expression cassette integrated into the pga locus compared to Bacillus licheniformis M409. Protease activity of Bacillus licheniformis mutants carrying the wildtype degU (grey) or the degU32 allele (dark grey) are indicated. The mutation introduced into either of the two strain backgrounds is indicated as ‘key mutation’ next to the corresponding strain number.

FIG. 4: Protease productivity of different Bacillus licheniformis strains. The relative protease activity in percent is plotted against the indicated Bacillus licheniformis strains for the 72 h timepoints of fed-batch cultivation with the Bacillus licheniformis strain PC36 set to 100% (grey design). Protease activity of Bacillus licheniformis mutants carrying an additional copy of the degQ gene under control of a strong constitutive promoter integrated into the cat locus (grey/white design). The mutation additionally introduced into the strain background is indicated as ‘key mutation’ next to the corresponding strain number.

FIG. 5: SDS-PAGE of supernatant from B. licheniformis strains after 72 h of simulated fed-batch cultivation.

1-3=control strain; 4=ForD deletion strain; 5-7=DegQ overexpression strain Overexpression of DegQ leads to loss of ForD from the extracellular proteome; M=Precision Plus Protein Standard with indicated size in kDa of selected bands; filled arrow-head highlights the ForD protein band.

FIG. 6: SDS-PAGE of supernatant from B. licheniformis protease expression strains after 72 h of simulated fed-batch cultivation.

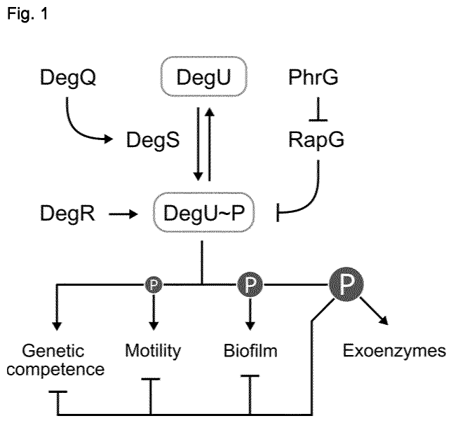

1-3=control strain; 4=ForD deletion strain; 5-7=DegQ overexpression strain; Overexpression of DegQ leads to loss of ForD from the extracellular proteome; M=Precision Plus Protein Standard with indicated size in kDa of selected bands; filled arrowhead highlights the ForD protein band; open arrowhead indicates the heterologous protease.

Table A contains an overview on genes that can be modified in accordance with the present invention. The table contains information on the function/activity of the genes. Further included are the sequences of the genes for Bacillus subtilis and Bacillus licheniformis, as well as the polypeptides encoded by said genes.

### DETAILED DESCRIPTION OF THE INVENTION

It is to be understood that as used in the specification and in the claims, “a” or “an” can mean one or more, depending upon the context in which it is used. Thus, for example, reference to “a cell” can mean that at least one cell can be utilized.

Further, it will be understood that the term “at least one” as used herein means that one or more of the items referred to following the term may be used in accordance with the invention. For example, if the term indicates that at least one feed solution shall be used this may be understood as one feed solution or more than one feed solutions, i.e. two, three, four, five or any other number of feed solutions. Depending on the item the term refers to the skilled person understands as to what upper limit the term may refer, if any.

The term “about” as used herein means that with respect to any number recited after said term an interval accuracy exists within in which a technical effect can be achieved. Accordingly, about as referred to herein, preferably, refers to the precise numerical value or a range around said precise numerical value of ±20%, preferably ±15%, more preferably ±10%, and even more preferably ±5%.

The term “comprising” as used herein shall not be understood in a limiting sense. The term rather indicates that more than the actual items referred to may be present, e.g., if it refers to a method comprising certain steps, the presence of further steps shall not be excluded. However, the term “comprising” also encompasses embodiments where only the items referred to are present, i.e. it has a limiting meaning in the sense of “consisting of”.

The terms “polynucleotide”, “nucleic acid sequence”, “nucleotide sequence”, “nucleic acid”, “nucleic acid molecule” are used interchangeably herein and refer to nucleotides, typically deoxyribonucleotides, in a polymeric unbranched form of any length. The terms “polypeptide” and “protein” are used interchangeably herein and refer to amino acids in a polymeric form of any length, linked together by peptide bonds.

The terms “coding for” and “encoding” are used interchangeably herein. Typically, the terms refer to the property of specific sequences of nucleotides in a polynucleotide, such as a gene, a cDNA, or an mRNA, to serve as templates for synthesis of other macromolecules such as a defined sequence of amino acids. Thus, a gene codes for a protein, if transcription and translation of mRNA corresponding to that gene produces the protein in a cell or other biological system.

The term “inactivating” a gene preferably, means that the expression of the gene has been reduced as compared to expression in a control cell. Preferably, expression of the gene in the bacterial host cell of the present invention has been reduced by at least 40% such as at least 50%, at least 60%, at least 70%, at least 80%, or at least 90% as compared to the corresponding expression in the control cell. More preferably, said expression has been reduced by at least 95%. Most preferably, it has been reduced by 100%, i.e. has been eliminated completely. The inactivation of a gene as referred to herein may be achieved by any method deemed appropriate. In an embodiment, the gene has been inactivated by mutation, i.e. by mutating the gene. Preferably, said mutation is a deletion, said gene has been deleted.

As used herein, the “deletion” of a gene refers to the deletion of the entire coding sequence, deletion of part of the coding sequence, or deletion of the coding sequence including flanking regions. The end result is that the deleted gene is effectively non-functional. In simple terms, a “deletion” is defined as a change in either nucleotide or amino acid sequence in which one or more nucleotides or amino acid residues, respectively, have been removed (i.e., are absent). Thus, a deletion strain has fewer nucleotides or amino acids than the respective wild-type organism.

Methods for generation of chromosomal gene deletion, substitution, mutation in Bacillus are known in the art. Examples for gene inactivation by means of homologous recombination are given in WO03083125 and Kostner et al. (Kostner D, Ehrenreich A. Microbiology (Reading). 2017 November; 163(11)), or by means of CRISPR/Cas technologies as described e.g. in WO2020206202 and WO2020206197.

Further, a gene may have been inactivated by gene silencing. Gene silencing can be achieved by introducing into said bacterial host cell antisense expression constructs that result in antisense RNAs complementary to the mRNA of the gene, thereby inhibiting expression of said genes. Alternatively, the expression of said gene can be inhibited by blocking transcriptional initiation or transcriptional elongation through the mechanism of CRISPR-inhibition (WO18009520).

Some of the genetic modifications introduced into the host cell shall cause increased expression of gene, such as of degU, degS, degQ and/or degR. Methods for increasing expression of genes or gene products are well documented in the art and include, for example, overexpression driven by appropriate promoters. In some embodiments, the increased expression is achieved by introducing and expressing the gene into the host cell (under control of a suitable promoter). For example, increased expression of degQ was achieved by introducing the degQ gene into the host cell and expressing said degQ gene. Thus, an additional copy of the degQ was introduced.

Furthermore, increased expression of a nucleic acid may be achieved by introducing isolated nucleic acids which serve as promoter in an appropriate position (typically upstream) of a gene so as to upregulate expression of a nucleic acid encoding gene. For example, endogenous promoters may be altered in vivo by mutation, deletion, and/or substitution, or isolated promoters may be introduced into a host cell in the proper orientation and distance from the nucleic acid encoding gene so as to increase the expression of the gene.

### The Host Cell

The term “host cell” in accordance with the present invention refers to a bacterial cell. In a preferred embodiment, the host cell belongs to the genus of Bacillus. For example, the host cell may be a Bacillus pumilus, Bacillus velezensis, Bacillus amyloliquefaciens, Bacillus alcalophilus, Bacillus licheniformis, Bacillus lentus, Bacillus clausii, Bacillus halodurans, Bacillus megaterium, Bacillus methanolicus, Bacillus globigii, or Bacillus subtilis host cell. In a preferred embodiment, the host cell is a Bacillus licheniformis host cell. For example, the host cell may be a host cell of the Bacillus licheniformis strain ATCC14580 (which is the same as DSM13, see Veith et al. “The complete genome sequence of Bacillus licheniformis DSM13, an organism with great industrial potential.” J. Mol. Microbiol. Biotechnol. (2004) 7:204-211). In another preferred embodiment, the host cell is a Bacillus subtilis host cell. For example, the host cell may be a host cell of the Bacillus subtilis strain NCIB 3610. In some embodiments, the host cell is not a Bacillus subtilis host cell with reduced degQ expression levels, preferably, wherein the Bacillus subtilis host cell does not comprise the mutation T to C at position-85 relative to the translational start codon of the deqQ gene, such as in Bacillus subtilis 168. Preferably, the Bacillus subtilis host cell is not Bacillus subtilis 168.

In another preferred embodiment, the host cell is a Bacillus velezensis host cell. For example, the host cell may be a host cell of the Bacillus velezensis strain FZB42.

In another preferred embodiment, the host cell is a Bacillus amyloliquefaciens host cell. For example, the host cell may be a host cell of the Bacillus amyloliquefaciens strain XH7. In another preferred embodiment, the host cell is a Bacillus pumilus host cell. For example, the host cell may be a host cell of the Bacillus pumilus strain DSM27.

In another preferred embodiment, the host cell is a Bacillus lentus host cell. For example, the host cell may be a host cell of the Bacillus lentus strain DSM9.

In another preferred embodiment, the host cell is a Bacillus alcalophilus host cell. For example, the host cell may be a host cell of the Bacillus alcalophilus strain ATCC27647.

In another preferred embodiment, the host cell is a Bacillus methanolicus host cell. For example, the host cell may be a host cell of the Bacillus methanolicus strain PB1 (DSM16454) or Bacillus methanolicus strain MGA3 (ATCC53907).

The Bacillus host cell of the present invention shall be a modified host cell. Specifically, the host shall comprise
i) at least one (i.e. one or more than one) genetic modification that leads to decreased production of components of biofilm (as compared to a control cell) and
ii) at least one (i.e. one or more than one) genetic modification that leads to increased levels of phosphorylated DegU (as compared to a control cell).

The terms “increased” and “enhanced” are used interchangeably herein and shall mean in the sense of the application an increase, preferably of at least 10%, at least 20%, at least 30%, at least 40%, at least 50%, at least 60%, at least 70%, at least 80%, at least 90% or 100%. The terms “decreased” and “reduced” are used interchangeably herein and shall mean in the sense of the application a reduction, preferably of at least 10%, at least 20%, at least 30%, at least 40%, at least 50%, at least 60%, at least 70%, at least 80%, at least 90%, or at least 95%. In some embodiments, the level of a gene product or its activity is reduced by 100%. Thus, the activity is eliminated completely.

Preferably, the increase or decrease is with respect to a control cell. A “control cell” as referred to herein is a control cell of the same species which does not carry the respective modification, preferably which differs from the host cell only in that it does not carry the respective modification. Thus, the control cell is an unmodified cell, such as a wild-type cell, i.e. an unmodified wildtype cell, preferably a Bacillus licheniformis cell, which does not carry the respective modification. Preferably, the control cell is a Bacillus licheniformis cell, which differs from the host cell only in that it does not carry the respective modification.

Formosin D (ForD, SEQ ID NO: 215) is a bacteriocin which is produced by B. licheniformis, but not by B. subtilis. It has been shown in the studies underlying the present invention that overexpression of DegQ leads to loss of ForD from the extracellular proteome. Thus, overexpression of DegQ in B. licheniformis would also allow for obtaining the polypeptide of interest with a high purity. As DegQ overexpression leads to increased levels of phosphorylated DegU, other genetic modifications which lead to increased levels of phosphorylated DegU might also lead to higher purity.

Accordingly, the modified Bacillus licheniformis host cell described herein, preferably, comprises reduced expression of the gene coding for Formosin D (ForD, bacteriocin), preferably compared to a Bacillus licheniformis control cell that does not comprise modification described herein. Preferably, the modified Bacillus licheniformis host cell comprises no modification in the endogenous forD gene, i.e., the modified Bacillus licheniformis host cell still comprises an unmodified endogenous forD gene. Preferably, the modified Bacillus licheniformis host cell described herein comprises a reduced expression of the gene coding for the Formosin D protein, preferably compared to a Bacillus licheniformis control cell that does not comprise modification described herein, wherein the Formosin D protein is encoded by a sequence having with increased preference at least 60%, at least 70%, at least 80%, at least 90%, at least 95%, at least 99%, or 100% identity to SEQ ID NO: 214. Preferably, the modified Bacillus licheniformis host cell described herein comprises a reduced expression of the gene coding for the Formosin D protein, preferably compared to a Bacillus licheniformis control cell that does not comprise modification described herein, wherein the Formosin D protein has with increased preference at least 60%, at least 70%, at least 80%, at least 90%, at least 95%, at least 99%, or 100% identity to SEQ ID NO: 215. Preferably, the modified Bacillus licheniformis host cell described herein comprises a reduced expression of the gene coding for the Formosin D protein, preferably compared to a Bacillus licheniformis control cell that does not comprise modification described herein, wherein the Formosin D protein has with increased preference at least 60%, at least 70%, at least 80%, at least 90%, at least 95%, at least 99%, or 100% identity to SEQ ID NO: 215.

Preferably, the modified Bacillus licheniformis host cell described herein produces a compound of interest, preferably a polypeptide of interest, with increased purify, preferably compared to a Bacillus licheniformis control cell that does not comprise modification described herein. Thus, preferably, the modified Bacillus licheniformis host cell described herein comprises an increased purity of the compound of interest, preferably compared to a Bacillus licheniformis control cell that does not comprise modification described herein. Preferably, the modified Bacillus licheniformis host cell described herein produces a compound of interest, preferably a polypeptide of interest, with reduced amount of Formosin D (ForD), preferably compared to a Bacillus licheniformis control cell that does not comprise modification described herein. Preferably, the modified Bacillus licheniformis host cell described herein produces a compound of interest, preferably a polypeptide of interest, with reduced amount of the Formosin D protein, preferably compared to a Bacillus licheniformis control cell that does not comprise modification described herein, wherein the Formosin D protein has with increased preference at least 60%, at least 70%, at least 80%, at least 90%, at least 95%, at least 99%, or 100% identity to SEQ ID NO: 215. Preferably, the modified Bacillus licheniformis host cell described herein comprises an increased production of the compound of interest, preferably compared to a Bacillus licheniformis control cell that does not comprise modification described herein. Preferably, the modified Bacillus licheniformis host cell described herein comprises an increased purity of the compound of interest, preferably with reduced amount of Formosin D, preferably compared to a Bacillus licheniformis control cell that does not comprise modification described herein and comprises an increased production of the compound of interest, preferably compared to a Bacillus licheniformis control cell that does not comprise modification described herein.

Genetic Modifications that Lead to Increased Levels of Phosphorylated DegU

The host cell shall comprise at least one (i.e. one or more than one) genetic modification that leads to increased levels of phosphorylated DegU. “Phosphorylated DegU” is herein also referred to as DegU-P.

Preferably, the genetic modification which increases the amount of phosphorylated DegU is a genetic modification selected from
u1) a genetic modification causing increased expression of at least one of the genes selected from the group consisting of degU, degS, degQ and degR,
u2) a genetic modification causing decreased expression of the rapG gene or increased expression of the phrG gene,
u3) a genetic modification stabilizing the DegU phosphorylation state, such as a degU32 mutation,
u4) a genetic modification increasing autophosphorylation activity of the DegS protein, such as a DegS-S76D mutation, and/or
u5) a genetic modification reducing phosphatase activity of the DegS protein, such as a degS200 mutation.

The transcriptional regulatory protein DegU is a member of the two-component regulatory system DegS/DegU which plays an important role in the transition growth phase. The DegU protein is well-known in the art. Preferably, the DegU protein is a Bacillus DegU-Protein.

In an embodiment, the DegU protein is from Bacillus licheniformis (or is a variant thereof). Said protein has a sequence as shown in SEQ ID NO: 173 (and is encoded by a polynucleotide comprising a sequence as shown in SEQ ID NO: 169). Typically, the variant comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99%, but less than 100% identical to SEQ ID NO: 173.

In an embodiment, the DegU protein is from Bacillus subtilis (or is a variant thereof). Said protein has a sequence as shown in SEQ ID NO: 163 (and is encoded by a polynucleotide comprising a sequence as shown in SEQ ID NO: 159). The term “variant” is defined elsewhere herein. Typically, the variant comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99%, but less than 100% identical to SEQ ID NO: 163.

Preferably, the DegU protein is from Bacillus licheniformis, preferably has a sequence as shown in SEQ ID NO: 173 or a variant thereof.

The term “Phosphorylated DegU” as used herein preferably refers to the DegU protein which is phosphorylated at a position corresponding to position 56 of SEQ ID NO: 163 or to position 56 of SEQ ID NO: 173.

The amount of DegU-P can be preferably increased by mutations in the DegS/DegU two-component regulatory system.

In an embodiment, the amount of DegU-P can be increased by a genetic modification causing increased expression of degU (or a variant thereof), degS (or a variant thereof), degQ (or a variant thereof) or degR (or a variant thereof). The term “DegU” has been defined above.

DegS is a signal transduction histidine-protein kinase/phosphatase. The kinase plays an important role in the transition growth phase and is involved in the control of expression of different cellular functions. The protein acts as both a protein kinase that undergoes autophosphorylation and transfers the phosphate to DegU.

In an embodiment, the DegS protein is from Bacillus licheniformis (or is a variant thereof). Said protein has a sequence as shown in SEQ ID NO: 172 (and is encoded by a polynucleotide comprising a sequence as shown in SEQ ID NO: 168). Typically, the variant comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99%, but less than 100% identical to SEQ ID NO: 172.

In an embodiment, the DegS protein is from Bacillus subtilis (or is a variant thereof). Said protein has a sequence as shown in SEQ ID NO: 162 (and is encoded by a polynucleotide comprising a sequence as shown in SEQ ID NO: 158). Typically, the variant comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99%, but less than 100% identical to SEQ ID NO: 162.

Preferably, the DegS protein is from Bacillus licheniformis, preferably has a sequence as shown in SEQ ID NO: 172 or a variant thereof.

DegQ is a degradation enzyme regulation protein which stimulates the phosphotransfer from phospho-DegS to DegU.

In an embodiment, the DegQ protein is from Bacillus licheniformis (or is a variant thereof). Said protein has a sequence as shown in SEQ ID NO: 170 (and is encoded by a polynucleotide comprising a sequence as shown in SEQ ID NO: 166). Typically, the variant comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99%, but less than 100% identical to SEQ ID NO: 170.

In an embodiment, the DegQ protein is from Bacillus subtilis (or is a variant thereof). Said protein has a sequence as shown in SEQ ID NO: 160 (and is encoded by a polynucleotide comprising a sequence as shown in SEQ ID NO: 156). Typically, the variant comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99%, but less than 100% identical to SEQ ID NO: 160.

Preferably, the DegQ protein is from Bacillus licheniformis, preferably has a sequence as shown in SEQ ID NO: 170 or a variant thereof.

DegR is regulatory protein which stabilizes the phosphorylated form of DegU. Typically, the stabilization contributes to increased levels of phosphorylated DegU and results in enhanced production of exoenzymes e.g. alkaline protease, and neutral protease.

In an embodiment, the DegR protein is from Bacillus licheniformis (or is a variant thereof). Said protein has a sequence as shown in SEQ ID NO: 171 (and is encoded by a polynucleotide comprising a sequence as shown in SEQ ID NO: 167). Typically, the variant comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99%, but less than 100% identical to SEQ ID NO: 171.

In an embodiment, the DegR protein is from Bacillus subtilis (or is a variant thereof). Said protein has a sequence as shown in SEQ ID NO: 161 (and is encoded by a polynucleotide comprising a sequence as shown in SEQ ID NO: 157). Typically, the variant comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99%, but less than 100% identical to SEQ ID NO: 161.

Preferably, the DegR protein is from Bacillus licheniformis, preferably has a sequence as shown in SEQ ID NO: 171 or a variant thereof.

In another embodiment, the amount of DegU-P is increased by a genetic modification stabilizing the DegU phosphorylation state, such as a degU32 mutation. The degU32 mutation, also referred to as DegU H12L hyperphosphorylation mutant, is well-known in the art. The mutant comprises a H12L substitution, i.e. the amino acid Histidine at position 12 is replaced with a Leucine. The degU32 mutation is herein also referred to as “DegU H12L mutation”. The mutation may be introduced by the using the CRISPR-Cas9 technology as described in the Examples section.

In an embodiment, the degU32 mutant polypeptide comprises an amino acid sequence as shown in SEQ ID NO: 165, or is a variant thereof. The term “variant” is defined elsewhere herein. Typically, the variant comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99%, but less than 100% identical to SEQ ID NO: 165. It is to be understood that the variant comprises the H12L substitution.

In another embodiment, the degU32 mutant polypeptide comprises an amino acid sequence as shown in SEQ ID NO: 175 or is a variant thereof. Typically, the variant comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99%, but less than 100% identical to SEQ ID NO: 175. It is to be understood that the variant comprises the H12L substitution.

Preferably, the degU32 mutant polypeptide comprises an amino acid sequence as shown in SEQ ID NO: 175, or is a variant thereof.

Preferably, the degU32 mutant polypeptide is expressed in the modified host cell. This can be achieved by introducing and expressing encoding the degU32 mutant polypeptide (or variant thereof) in the host cell. Preferably, the polynucleotide is operably linked to a suitable promoter. The term “promoter” is defined elsewhere herein. Also preferably, the degU32 mutation is introduced into the chromosomal degU gene so that the degU32 gene is expressed within the native degS/degU operon and its transcriptional and translational regulation. This can be e.g. achieved by using the CRISPR-Cas9 technology.

As set forth above, the mutation which increases the amount of phosphorylated DegU may be also a mutation causing decreased expression of rapG or increased expression of phrG. In an embodiment, the RapG protein is from Bacillus licheniformis (or is a variant thereof). Said protein has a sequence as shown in SEQ ID NO: 153 (and is encoded by a polynucleotide comprising a sequence as shown in SEQ ID NO: 130). Typically, the variant comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99%, but less than 100% identical to SEQ ID NO: 153.

In an embodiment, the RapG protein is from Bacillus subtilis (or is a variant thereof). Said protein has a sequence as shown in SEQ ID NO: 107 (and is encoded by a polynucleotide comprising a sequence as shown in SEQ ID NO: 84). Typically, the variant comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99%, but less than 100% identical to SEQ ID NO: 107.

Preferably, the RapG protein is from Bacillus licheniformis, preferably has a sequence as shown in SEQ ID NO: 153 or a variant thereof.

In an embodiment, the PhrG peptide is from Bacillus licheniformis (or is a variant thereof). Said protein has a sequence as shown in SEQ ID NO: 152 (and is encoded by a polynucleotide comprising a sequence as shown in SEQ ID NO: 129). Typically, the variant comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99%, but less than 100% identical to SEQ ID NO: 152.

In an embodiment, the PhrG peptide is from Bacillus subtilis (or is a variant thereof). Said protein has a sequence as shown in SEQ ID NO: 106 (and is encoded by a polynucleotide comprising a sequence as shown in SEQ ID NO: 83). Typically, the variant comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99%, but less than 100% identical to SEQ ID NO: 106.

Preferably, the PhrG protein is from Bacillus licheniformis, preferably has a sequence as shown in SEQ ID NO: 152 or a variant thereof.

As set forth above, the mutation which increases the amount of phosphorylated DegU may be also a genetic modification increasing autophosphorylation activity of the DegS protein, such as a DegS-X76D, preferably DegS-S76D mutation.

The DegS-X76D mutant comprises a X76D substitution, i.e. a substitution at position 76 according to the numbering of SEQ ID NO: 172 to Aspartic Acid, preferably the amino acid Serine at position 76 is replaced with Aspartic Acid at amino acid position 76 of the DegS protein according to the numbering of SEQ ID NO: 172. In an embodiment, the DegS-X76D mutant polypeptide comprises an amino acid sequence as shown in SEQ ID NO: 164 or 174, preferably SEQ ID NO: 174, or is a variant thereof. Typically, the variant comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99%, but less than 100% identical to SEQ ID NO: 164 or 174, preferably SEQ ID NO: 174. It is to be understood that the variant comprises the X76D substitution, preferably S76D substitution. Preferably, the mutant polypeptide is expressed in the modified host cell. This can be achieved by introducing and expressing encoding the mutant polypeptide (or variant thereof) in the host cell. Preferably, the polynucleotide is operably linked to a suitable promoter.

As set forth above, the mutation which increases the amount of phosphorylated DegU may be also a genetic modification reducing phosphatase activity of the DegS protein, such as a degS200 mutation. DegS has been defined above.

The degS200 mutation, also referred to as DegS-X218E mutant, comprises a X218E, preferably a G218E substitution, i.e. a substitution at position 218 according to the numbering of SEQ ID NO: 172 to Glutamic Acid, preferably the amino acid Glycine is replaced with Glutamic Acid at position 218 of the DegS protein according to the numbering of SEQ ID NO: 172. In an embodiment, the DegS-G218E mutant polypeptide comprises an amino acid sequence as shown in SEQ ID NO: 185 or 186, preferably SEQ ID NO: 186, or is a variant thereof. Typically, the variant comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99%, but less than 100% identical to SEQ ID NO: 185 or 186, preferably SEQ ID NO: 186. It is to be understood that the variant comprises the X218E substitution, preferably G218E substitution. It is understood within the scope of the invention that an amino acid exchange X218D, preferably G218D, would lead to the same effect. Preferably, the mutant polypeptide is expressed in the modified host cell. This can be achieved by introducing and expressing encoding the mutant polypeptide (or variant thereof) in the host cell. Preferably, the polynucleotide is operably linked to a suitable promoter.

In an embodiment, the host cell comprises two genetic modifications which increase the amount of phosphorylated DegU. For example, the host cell comprises a genetic modification causing increased expression of degQ and a degU32 mutation.

Genetic Modifications that Lead to Decreased Levels of Exopolymeric Substances (EPS) and/or TasA

The host cell as set forth herein shall comprise a mutation which reduces the amount of exopolymeric substances (EPS) and/or a mutation which reduces the amount of the biofilm extracellular matrix component TasA as compared to a control cell.

Bacterial exopolymeric substances (EPS) are molecules released in response to the physiological stress encountered in the natural environment. Exopolymeric substances are structural components of the extracellular matrix in which cells are embedded during biofilm development.

The TasA polypeptide is a major component of biofilm matrix. It forms amyloid fibers. In an embodiment, the TasA polypeptide is the Bacillus subtilis TasA polypeptide (or a variant thereof).

In an embodiment, the TasA polypeptide is the Bacillus licheniformis TasA polypeptide (or a variant thereof). The Bacillus licheniformis TasA polypeptide comprises an amino acid sequence as shown in SEQ ID NO: 151. It is encoded by a polynucleotide comprising a nucleic sequence as shown in SEQ ID NO: 128. Typically, the variant comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99%, but less than 100% identical to SEQ ID NO: 151.

The Bacillus subtilis TasA polypeptide comprises an amino acid sequence as shown in SEQ ID NO: 105. It is encoded by a polynucleotide comprising a nucleic sequence as shown in SEQ ID NO: 82. Typically, the variant comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99%, but less than 100% identical to SEQ ID NO: 105.

Preferably, the TasA protein is from Bacillus licheniformis, preferably has a sequence as shown in SEQ ID NO: 151 or a variant thereof.

Preferably, the mutation which reduces the amount of the biofilm extracellular matrix component TasA is a mutation that causes a reduced expression of the tapA-sipW-tasA operon. Said reduced expression may be achieved by inactivating the tapA-sipW-tasA operon, or a portion thereof. For example, tapA-sipW-tasA operon, or a portion thereof, may be deleted. A portion of the operon may be the tasA gene, the sipW gene or the tapA gene. The nucleic acid sequences as well as the amino acid sequences of the aforementioned genes/polypeptides are shown in Table A (for Bacillus licheniformis and Bacillus subtilis).

Preferably, the mutation which reduces the amount of exopolymeric substances (EPS) is a mutation that causes a reduced expression of the epsA-O operon. The epsA-O operon comprises the following genes: epsA, epsB, epsC, epsD, epsE, epsF, epsG, epsH, epsI, epsJ, epsK, epsL, epsM, epsN, and epsO. The nucleic acid sequences as well as the amino acid sequences of the aforementioned genes/polypeptides are shown in Table A (for Bacillus licheniformis and Bacillus subtilis). Preferably, the mutation that inactivates the epsA-O operon is a deletion of epsA-O operon, or of a portion thereof. In some embodiments, at least one gene selected from epsA, epsB, epsC, epsD, epsE, epsF, epsG, epsH, epsI, epsJ, epsK, epsL, epsM, epsN, and epsO is deleted. Preferably, at least one gene selected from epsA, epsB, epsC, epsD, epsE, epsF, epsG, epsH, epsI, epsJ, epsK, epsL, epsM, epsN, and epsO is deleted. More preferably, at least one gene selected from epsD, epsE, epsK, epsM and epsN is deleted. Most preferably, at least the epsE gene is deleted.

More preferably, at least 6 genes of the aforementioned genes, i.e. at least 6 genes selected from epsA, epsB, epsC, epsD, epsE, epsF, epsG, epsH, epsI, epsJ, epsK, epsL, epsM, epsN, and epsO are deleted (preferably, at least one of the at least 6 genes is the epsE gene). In some embodiments, the entire epsA-O operon is deleted.

Alternatively or additionally, the genetic modification that leads to decreased levels of exopolymeric substances (EPS) and/or TasA may be a mutation that inactivates the gene remA. Thus, it is envisaged to inactivate said gene. Without being bound to theory, the present inventors believe that a reduction of function of the RemA protein in the Bacillus host cell leads to an increased production of a compound of interest by the Bacillus host cell. Thus, preferably, the host comprises an altered RemA protein, preferably wherein the alteration of the RemA protein confers a loss of RemA-mediated transcription activation. Preferably, the alteration of the RemA protein confers a reduced DNA binding affinity of the RemA protein. For example, said gene, or a portion thereof, may be deleted in order to inactivate the protein The nucleic acid sequence as well as the amino acid sequence of the aforementioned genes/polypeptides are shown in Table A [for Bacillus licheniformis (amino acid sequence SEQ ID NO: 154) and Bacillus subtilis (amino acid sequence SEQ ID NO: 108).

Alternatively or additionally, the genetic modification that leads to decreased levels of exopolymeric substances (EPS) and/or TasA may be a mutation that inactivates the gene remB. Thus, it is envisaged to inactivate said gene. For examples, said gene, or a portion thereof, may be deleted. The nucleic acid sequence as well as the amino acid sequence of the aforementioned genes/polypeptides are shown in Table A (for Bacillus licheniformis and Bacillus subtilis). In an embodiment, the RemA polypeptide is the Bacillus licheniformis RemA polypeptide (or a variant thereof).

The Bacillus licheniformis RemA polypeptide comprises an amino acid sequence as shown in SEQ ID NO: 154. It is encoded by a polynucleotide comprising a nucleic sequence as shown in SEQ ID NO: 131. Typically, the variant comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99%, but less than 100% identical to SEQ ID NO: 154.

The Bacillus subtilis RemA polypeptide comprises an amino acid sequence as shown in SEQ ID NO: 108. It is encoded by a polynucleotide comprising a nucleic sequence as shown in SEQ ID NO: 85. Typically, the variant comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99%, but less than 100% identical to SEQ ID NO: 108.

Preferably, the RemA protein is from Bacillus licheniformis, preferably has a sequence as shown in SEQ ID NO: 154 or a variant thereof.

In an embodiment, the RemB polypeptide is the Bacillus licheniformis RemB polypeptide (or a variant thereof). The Bacillus licheniformis RemB polypeptide comprises an amino acid sequence as shown in SEQ ID NO: 207 It is encoded by a polynucleotide comprising a nucleic sequence as shown in SEQ ID NO: 206. Typically, the variant comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99%, but less than 100% identical to SEQ ID NO: 207.

The Bacillus subtilis RemB polypeptide comprises an amino acid sequence as shown in SEQ ID NO: 205. It is encoded by a polynucleotide comprising a nucleic sequence as shown in SEQ ID NO: 204. Typically, the variant comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99%, but less than 100% identical to SEQ ID NO: 205.

Preferably, the RemB protein is from Bacillus licheniformis, preferably has a sequence as shown in SEQ ID NO: 207 or a variant thereof.

In one embodiment, the alteration of the RemA protein is caused by one or more point mutations, in the gene coding for the RemA protein. Preferably, the one or more point mutations in the gene coding for the RemA protein are selected from the group consisting of missense mutations, nonsense mutation, and frame-shift mutations. Preferably, the one or more point mutations in the gene coding for the RemA protein is one or more missense mutations. Preferably, the one or more point mutations in the remA gene result in an inactivation of the RemA protein in the Bacillus host cell.

Preferably, the one or more missense point mutations in the gene coding for the RemA protein are at positions coding for conserved amino acids in the RemA protein. A conserved amino acid position in a protein can also be described as a position having an IC value equal or greater 2.0. The IC (Information Content) value as used herein is the computed value R_Sequence (I) as is described by Schneider, T. D.; Stephens, R. M. Sequence Logos: A New Way to Display Consensus Sequences. Nucleic Acids Res. 1990, 18 (20), 6097-6100, with using 20 states for amino acid sequences. Preferably, the one or more missense point mutations in the gene coding for the RemA protein are at positions coding conserved amino acid positions of SEQ ID NO: 154, with an IC value equal or greater than 2.0, preferably equal or greater than 2.5, more preferably equal or greater than 3.0, or even more preferably equal or greater than 3.2, most preferably equal or greater than 3.5. Preferably, the one or more missense point mutations in the gene coding for the RemA protein are at positions coding for conserved amino acid positions of SEQ ID NO: 154 with an IC value equal or greater than 3.2, most preferably equal or greater than 3.5.

Preferably, the one or more point mutations in the gene coding for the RemA protein result in non-conservative amino acid substitutions (as defined herein, see, e.g., Table 10) in the RemA protein. Thus, preferably the altered RemA protein comprises one or more non-conservative amino acid exchanges. Preferably the altered RemA protein comprises one or more nonconservative amino acid exchanges that lead to a reduced function of the RemA protein in the Bacillus cell. Preferably the altered RemA protein comprises one or more non-conservative amino acid exchanges that lead to an inactivation of the RemA protein in the Bacillus cell. Preferably, the one or more point mutations in the gene coding for the RemA protein result in nonconservative amino acid substitutions, preferably inactivating substitutions, at conserved amino acid positions of SEQ ID NO: 154 with an IC value equal or greater than 2.0, preferably equal or greater than 2.5, more preferably equal or greater than 3.0, or even more preferably equal or greater than 3.2, most preferably equal or greater than 3.5. Preferably, the one or more missense point mutations in the gene coding for the RemA protein are at positions coding for conserved amino acid positions of SEQ ID NO: 154 with an IC value equal or greater than 3.2, most preferably equal or greater than 3.5. Preferably, the one or more point mutations in the gene coding for the RemA protein result in non-conservative amino acid substitutions, preferably as shown in Table 10, preferably inactivating substitutions, at conserved amino acid positions of SEQ ID NO: 154 with an IC value equal or greater than 3.0, or even more preferably equal or greater than 3.2, most preferably equal or greater than 3.5.

Preferably, the one or more point mutations in the gene coding for the RemA protein result in non-conservative amino acid substitutions, preferably inactivating substitutions, at one or more of amino acid positions corresponding to amino acid positions 5-77 of SEQ ID NO: 154, preferably at one or more amino acid positions corresponding to amino acid positions selected from the group consisting of 18, G9, F10, G11, N12, R18, S27, P29, K31, R32, D45, T47, G49, R50, T52, D59, L65, S66, T72, and R76 of SEQ ID NO: 154, most preferably at one or more amino acid position selected from amino acid positions corresponding to R18 and P29 of SEQ ID NO: 154. Preferably the altered RemA protein comprises at least one of the substitutions X18W and X29S at amino acid position R18 and P29 of SEQ ID NO: 154. Preferably the altered RemA protein comprises the substitutions X18W and X29S at amino acid position R18 and P29 of SEQ ID NO: 154.

The term “amino acid positions corresponding to amino acid positions” followed by certain amino acid positions indicated by number or residue and number of SEQ ID NO: 154 shall mean that for referring to certain amino acid positions in a particular RemA protein a sequence alignment is made with SEQ ID NO: 154 and the amino acid numbering of SEQ ID NO: 154 at a certain amino acid position is used for reference (i.e., according to the numbering of SEQ ID NO: 154).

Preferably, the altered RemA protein comprises an amino acid sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% identity to SEQ ID NO: 154, or 108. Preferably, the altered RemA protein comprises an amino acid sequence having at least 80%, at least 85%, at least 90%, at least 91%, at least 92%, at least 93%, at least 94%, at least 95%, at least 96%, at least 97%, at least 98%, or at least 99%, but less than 100% identity to SEQ ID NO: 154 or 108, preferably SEQ ID NO: 154.

Preferably, the altered RemA protein comprises an amino acid sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% identity to SEQ ID NO: 154 or 108, preferably SEQ ID NO: 154 and one or more amino acid substitutions, preferably one or more non-conservative amino acid substitutions, preferably inactivating substitutions, at one or more of amino acid positions corresponding to amino acid positions 5-77 of SEQ ID NO: 154, preferably at one or more amino acid positions corresponding to amino acid positions selected from the group consisting of 18, G9, F10, G11, N12, R18, S27, P29, K31, R32, D45, T47, G49, R50, T52, D59, L65, S66, T72, and R76 of SEQ ID NO: 154, most preferably at one or more, preferably both, amino acid position selected from amino acid positions corresponding to R18 and P29 of SEQ ID NO: 154. Preferably, the altered RemA protein comprises an amino acid sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% identity to SEQ ID NO: 154 and one or more nonconservative amino acid substitutions, preferably inactivating substitutions, at one or more of amino acid positions corresponding to amino acid positions 5-77 of SEQ ID NO: 154, preferably at one or more amino acid positions corresponding to amino acid positions selected from the group consisting of 18, G9, F10, G11, N12, R18, S27, P29, K31, R32, D45, T47, G49, R50, T52, D59, L65, S66, T72, and R76 of SEQ ID NO: 154, most preferably at one or more amino acid position selected from amino acid positions corresponding to R18 and P29 of SEQ ID NO: 154. Preferably the altered RemA protein comprises at least one of the substitutions X18W and X29S at amino acid position R18 and P29 of SEQ ID NO: 154. Preferably the altered RemA protein comprises the substitutions X18W and X29S at amino acid position R18 and P29 of SEQ ID NO: 154. Preferably the altered RemA protein comprises an amino acid sequence having at least 80%, at least 85%, at least 90%, at least 91%, at least 92%, at least 93%, at least 94%, at least 95%, at least 96%, at least 97%, at least 98%, or at least 99%, but less than 100% identity to SEQ ID NO: 154 and comprises at least one, preferably both, of the substitutions R18W and P29S at amino acid position R18 and P29 of SEQ ID NO: 154.

Alternatively or additionally, the genetic modification that leads to decreased levels of exopolymeric substances (EPS) and/or TasA may be a mutation that inactivates the slrA gene. Thus, it is envisaged to inactivate said gene. For example, said gene, or a portion thereof, may be deleted. Alternatively, the mutation that inactivates the slrA gene, is a missense mutation of the slrA gene. The nucleic acid sequences as well as the amino acid sequences of the aforementioned genes/polypeptides are shown in Table A (for Bacillus licheniformis and Bacillus subtilis). In a preferred embodiment, the host cell of the present invention comprises at least the following modifications: a DegU H12L mutation and a remA or remB, preferably remA, missense mutation.

In a preferred embodiment, the host cell of the present invention comprises at least the following modifications: a DegU H12L mutation and a mutation that inactivates the sirA gene (such as a deletion of the sirA gene).

In a preferred embodiment, the host cell of the present invention comprises at least the following modifications: a DegU H12L mutation, and a mutation that inactivates the epsA-O operon (such as a deletion of epsA-O operon, or of a portion thereof).

In a preferred embodiment, the host cell of the present invention comprises at least the following modifications: a DegU H12L mutation, and a mutation that inactivates the tapA-sipW-tasA operon (such as a deletion of tapA-sipW-tasA operon, or of a portion thereof).

In a preferred embodiment, the host cell of the present invention comprises at least the following modifications: a DegU H12L mutation, a mutation that inactivates the epsA-O operon (such as a deletion of epsA-O operon, or of a portion thereof), and a mutation that inactivates the tapA-sipW-tasA operon (such as a deletion of tapA-sipW-tasA operon, or of a portion thereof). In a preferred embodiment, the host cell of the present invention comprises at least the following modifications: a DegU H12L mutation a mutation causing increased expression of degQ (such as the introduction and expression of an additional copy of the degQ gene) and a mutation that inactivates the epsA-O operon (such as a deletion of epsA-O operon, or of a portion thereof). Said host cell may comprise one or more further modifications such as a modification that inactivates the tapA-sipW-tasA operon, a remA modification, a remB modification, and/or a slrA modification as described herein. Preferably, said host cell may comprise one or more further modifications such as a modification that inactivates the tapA-sipW-tasA operon, a remA modification, and/or a slrA modification as described herein.

In a preferred embodiment, the host cell of the present invention comprises at least the following modifications: a DegU H12L mutation, and a mutation that inactivates the tapA-sipW-tasA operon (such as a deletion of tapA-sipW-tasA operon, or of a portion thereof).

In a preferred embodiment, the host cell of the present invention comprises at least the following modifications: a DegU H12L mutation, a mutation causing increased expression of degQ (such as the introduction and expression of an additional copy of the degQ gene), and a mutation that inactivates the tapA-sipW-tasA operon (such as a deletion of tapA-sipW-tasA operon, or of a portion thereof). Said host cell may comprise one or more further modifications such as a modification that inactivates the tapA-sipW-tasA operon, the epsA-O operon, a remA modification, a remB modification and/or slrA modification as described herein. Preferably, said host cell may comprise one or more further modifications such as a modification that inactivates the tapA-sipW-tasA operon, the epsA-O operon, a remA modification, and/or slrA modification as described herein.

In a preferred embodiment, the host cell of the present invention comprises at least the following modifications: a mutation causing increased expression of degQ (such as the introduction and expression of an additional copy of the degQ gene), and a remA or remB missense mutation. Preferably, the host cell of the present invention comprises at least the following modifications: a mutation causing increased expression of degQ (such as the introduction and expression of an additional copy of the degQ gene), and a remA missense mutation.

In a preferred embodiment, the host cell of the present invention comprises at least the following modifications: a mutation causing increased expression of degQ (such as the introduction and expression of an additional copy of the degQ gene), a mutation that inactivates the epsA-O operon (such as a deletion of epsA-O operon, or of a portion thereof), a mutation that inactivates the tapA-sipW-tasA operon (such as a deletion of tapA-sipW-tasA operon, or of a portion thereof).

In a preferred embodiment, the host cell of the present invention comprises at least the following modifications: a mutation causing increased expression of degQ (such as the introduction and expression of an additional copy of the degQ), and a mutation that inactivates the epsA-O operon (such as a deletion of epsA-O operon, or of a portion thereof).

In a preferred embodiment, the host cell of the present invention comprises at least the following modifications: a mutation causing increased expression of degQ (such as the introduction and expression of an additional copy of the degQ), and a mutation that inactivates the tapA-sipW-tasA operon (such as a deletion of tapA-sipW-tasA operon, or of a portion thereof). Moreover, the host cell may comprise a polynucleotide encoding a polypeptide of interest (as described elsewhere herein in more detail).

Furthermore, it is envisaged that the modified host cell as set forth herein does not produce poly-gamma-glutamate (pga) or produces a reduced amount of pga. Accordingly, at least one gene involved in poly-gamma-glutamate (pga) production has been inactivated (such as deleted). Preferably, the at least one gene involved in poly-gamma-glutamate (pga) is at least one gene selected from ywsC (pgsB), ywtA (pgsC), ywtB (pgsA) and ywtC (pgsE). Preferably, all aforementioned genes, i.e. ywsC (pgsB), ywtA (pgsC), ywtB (pgsA) and ywtC (pgsE) have been inactivated (such as deleted).

Furthermore, it is envisaged that the modified host cell is not capable to sporulate. This may be achieved by inactivating (such as deleting) at least one gene involved in sporulation. Genes involved in sporulation are well known in the art (EP1391502), comprising but not limited to sigE, sigF, spolIGA, spolIE, sigG, spolVCB, yqfD. In a preferred embodiment, the sigF gene is deleted.

### Polypeptide of Interest

The host cell of the present invention shall further comprise at least one polynucleotide encoding a polypeptide of interest (operably linked to a promoter).

The term “polypeptide of interest” as used herein refers to any protein, peptide or fragment thereof which is intended to be produced in the bacterial host cell. A protein, thus, encompasses polypeptides, peptides, fragments thereof as well as fusion proteins and the like.

Preferably, the polypeptide of interest is an enzyme, such as an exoenzyme. An exoenzyme (or extracellular enzyme), is an enzyme that is secreted by the host cell.

In a particularly preferred embodiment, the enzyme is classified as an oxidoreductase (EC 1), a transferase (EC 2), a hydrolase (EC 3), a lyase (EC 4), an isomerase (EC 5), or a ligase (EC 6). In a preferred embodiment, the protein of interest is an enzyme suitable to be used in detergents, feed and food applications.

Most preferably, the enzyme is a hydrolase (EC 3), preferably, a glycosidase (EC 3.2) or a peptidase (EC 3.4). Especially preferred enzymes are enzymes selected from the group consisting of an amylase (in particular an alpha-amylase (EC 3.2.1.1), beta-beta amylase (EC 3.2.1.2), a cellulase (EC 3.2.1.4), an endo-1,3-beta-xylanase xylanase (EC 3.2.1.32), an endo-1,4-beta-xylanase (EC 3.2.1.8), a lactase (EC 3.2.1.108), a galactosidase (EC 3.2.1.23 and EC 3.2.1.24), a mannanase (EC 3.2.1.24 and EC 3.2.1.25), a lipase (EC 3.1.1.3), a phytase (EC 3.1.3.8), a nuclease (EC 3.1.11 to EC 3.1.31), and a protease (EC 3.4); in particular an enzyme selected from the group consisting of amylase, protease, lipase, mannanase, phytase, xylanase, phosphatase, ß-galactosidase, lactase glucoamylase, nuclease, and cellulase, preferably, amylase, mannanase, lactase or protease, preferably, an amylase and a protease. Most preferred is a serine protease (EC 3.4.21), preferably a subtilisin protease.

In particular, the following proteins of interest are preferred:

Enzymes having proteolytic activity are called “proteases” or “peptidases”. Proteases are active proteins exerting “protease activity” or “proteolytic activity”. Proteases are members of class EC 3.4. Proteases include aminopeptidases (EC 3.4.11), dipeptidases (EC 3.4.13), dipeptidylpeptidases and tripeptidyl-peptidases (EC 3.4.14), peptidyl-dipeptidases (EC 3.4.15), serinetype carboxypeptidases (EC 3.4.16), metallocarboxypeptidases (EC 3.4.17), cysteine-type carboxypeptidases (EC 3.4.18), omega peptidases (EC 3.4.19), serine endopeptidases (EC 3.4.21), cysteine endopeptidases (EC 3.4.22), aspartic endopeptidases (EC 3.4.23), metalloendopeptidases (EC 3.4.24), threonine endopeptidases (EC 3.4.25), endo-peptidases of unknown catalytic mechanism (EC 3.4.99). Commercially available protease enzymes include but are not limited to Lavergy™ Pro (BASF); Alcalase®, Blaze®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coro-Nase®, Coronase® Ultra, Neutrase®, Everlase® and Esperase® (Novozymes A/S), those sold under the tradename Maxatase®, Maxacal®,
Maxapem®, Purafect®, Purafect® Prime, Pura-fect MAR, Purafect Ox®, Purafect OxP®, Puramax®, Properase®, FN2®, FN3®, FN4®, Ex-Cellase®, Eraser®, Ultimase®, Opticlean®, Effectenz®, Preferenz® and Optimase® (Dan-isco/DuPont), Axapem™ (Gist-Brocases N.V.), Bacillus lentus Alkaline Protease, and KAP (Bacillus alkalophilus subtilisin) from Kao. At least one protease may be selected from serine proteases (EC 3.4.21). Serine proteases or serine peptidases (EC 3.4.21) are characterized by having a serine in the catalytically active site, which forms a covalent adduct with the substrate during the catalytic reaction. A serine protease may be selected from the group consisting of chymotrypsin (e.g., EC 3.4.21.1), elastase (e.g., EC 3.4.21.36), elastase (e.g., EC 3.4.21.37 or EC 3.4.21.71), granzyme (e.g., EC 3.4.21.78 or EC 3.4.21.79), kallikrein (e.g., EC 3.4.21.34, EC 3.4.21.35, EC 3.4.21.118, or EC 3.4.21.119,) plasmin (e.g., EC 3.4.21.7), trypsin (e.g., EC 3.4.21.4), thrombin (e.g., EC 3.4.21.5,) and subtilisin (also known as subtilopeptidase, e.g., EC 3.4.21.62), the latter hereinafter also being referred to as “subtilisin”. Preferably, the protease is a protease variant of Bacillus lentus alkaline protease (BLAP), preferably BLAP comprising the substitution R101E (according to BPN′ numbering). Proteases according to the invention have proteolytic activity. The methods for determining proteolytic activity are well-known in the literature (see e.g. Gupta et al. (2002), Appl. Microbiol. Bio-technol. 60: 381-395).

In an embodiment, the polynucleotide encoding at least one polypeptide of interest is heterologous to the bacterial host cell. The term “heterologous” (or exogenous or foreign or recombinant or non-native) polypeptide or protein as used throughout the specification is defined herein as a polypeptide or protein that is not native to the host cell. Similarly, the term “heterologous” (or exogenous or foreign or recombinant or non-native) polynucleotide refers to a polynucleotide that is not native to the host cell.

In an embodiment, the at least one polynucleotide encoding a polypeptide of interest is present on a plasmid. The term “plasmid” refers to an extrachromosomal circular DNA, i.e. a vector that is autonomously replicating in the host cell. Thus, a plasmid is understood as extrachromosomal vector.

In another embodiment, the at least one polynucleotide encoding a polypeptide of interest is stably integrated into the bacterial chromosome.

### Promoter

The at least one polynucleotide encoding a polypeptide of interest shall be operably linked to a promoter.

The term “operably linked” as used herein refers to a functional linkage between the promoter sequence and the polynucleotide encoding a polypeptide of interest, such that the promoter sequence is able to initiate transcription of the polynucleotide encoding a polypeptide of interest (herein also referred to as gene of interest).

A “promoter” or “promoter sequence” is a nucleotide sequence located upstream of a gene on the same strand as the gene that enables that gene's transcription. A promoter is followed by the transcription start site of the gene. A promoter is recognized by RNA polymerase (together with any required transcription factors), which initiates transcription. A functional fragment or functional variant of a promoter is a nucleotide sequence which is recognizable by RNA polymerase, and capable of initiating transcription.

An “active promoter fragment”, “active promoter variant”, “functional promoter fragment” or “functional promoter variant” describes a fragment or variant of the nucleotide sequences of a promoter, which still has promoter activity.

A promoter can be an “inducer-dependent promoter” or an “inducer-independent promoter” comprising constitutive promoters or promoters which are under the control of other cellular regulating factors.

The person skilled in the art is capable to select suitable promoters for expressing the third alanine racemase and the polypeptide of interest. For example, the polynucleotide encoding the polypeptide of interest is, preferably, operably linked to an “inducer-dependent promoter” or an “inducer-independent promoter”. Further, the polynucleotide encoding the third alanine racemase is, preferably, operably linked to an “inducer-independent promoter”, such as a constitutive promoter.

An “inducer dependent promoter” is understood herein as a promoter that is increased in its activity to enable transcription of the gene to which the promoter is operably linked upon addition of an “inducer molecule” to the fermentation medium. Thus, for an inducer-dependent promoter, the presence of the inducer molecule triggers via signal transduction an increase in expression of the gene operably linked to the promoter. The gene expression prior activation by the presence of the inducer molecule does not need to be absent, but can also be present at a low level of basal gene expression that is increased after addition of the inducer molecule. The “inducer molecule” is a molecule, the presence of which in the fermentation medium is capable of affecting an increase in expression of a gene by increasing the activity of an inducer-dependent promoter operably linked to the gene. Preferably, the inducer molecule is a carbohydrate or an analog thereof. In one embodiment, the inducer molecule is a secondary carbon source of the Bacillus cell. In the presence of a mixture of carbohydrates, cells selectively take up the carbon source that provide them with the most energy and growth advantage (primary carbon source). Simultaneously, they repress the various functions involved in the catabolism and uptake of the less preferred carbon sources (secondary carbon source). Typically, a primary carbon source for Bacillus is glucose and various other sugars and sugar derivates being used by Bacillus as secondary carbon sources. Secondary carbon sources include e.g. mannose or lactose without being restricted to these.

Examples of inducer dependent promoters are given in the table below by reference to the respective operon:

| Operon | Regulator a) | Type b) | Inducer | Organism |
|---|---|---|---|---|
| sacPA | SacT | AT | sucrose | B. subtilis |
| sacB | SacY | AT | sucrose | B. subtilis |
| bgl PH | LicT | AT | β-glucosides | B. subtilis |
| licBCAH | LicR | A | oligo-β-glucosides | B. subtilis |
| levDEFG sacL | LevR | A | fructose | B. subtilis |
| mtlAD | MtlR | A | mannitol | B. subtilis |
| manPA-yjdF | ManR | A | mannose | B. subtilis |
| manR | ManR | A | mannose | B. subtilis |
| bglFB bglG | BglG | AT | β-glucosides | E. coli |
| lacTEGF | LacT | AT | lactose | L. casei |
| lacZYA | lacI | R | Allolactose; IPTG | E. coli |
| | | | (Isopropyl β-D-1- | |
| | | | thiogalactopy- | |
| | | | ranoside) | |
| araBAD | araC | AR | L-arabinose | E. coli |
| xylAB | XylR | R | Xylose | B. subtilis |

a: transcriptional regulator protein
b: A: activator
AT: antiterminator
R: repressor
AR: activator/repressor

In contrast thereto, the activity of promoters that do not depend on the presence of an inducer molecule (herein called ‘inducer-independent promoters’) are either constitutively active or can be increased regardless of the presence of an inducer molecule that is added to the fermentation medium.

Constitutive promoters are independent of other cellular regulating factors and transcription initiation is dependent on sigma factor A (sigA). The sigA-dependent promoters comprise the sigma factor A specific recognition sites ‘-35’-region and ‘-10’-region.

Preferably, the ‘inducer-independent promoter’ sequence is selected from the group consisting of constitutive promoters not limited to promoters Pveg, PlepA, PserA, PymdA, Pfba and derivatives thereof with different strength of gene expression (Guiziou et al, (2016): Nucleic Acids Res. 44(15), 7495-7508), the aprE promoter of Subtilisin encoding aprE gene of Bacilli, the bacteriophage SPO1 promoters P4, P5, P15 (WO15118126), the cryIIIA promoter from Bacillus thuringiensis (WO9425612), the amyQ promoter from Bacillus amyloliquefaciens, the amyL promoter and promoter variants from Bacillus licheniformis (U.S. Pat. No. 5,698,415) and combinations thereof, or active fragments or variants thereof, preferably an aprE promoter sequence. In a preferred embodiment, the promoter which is operably linked to the polynucleotide encoding the polypeptide of interest is a DegU regulated promoter. Accordingly, the promoter shall be a promoter which is activated by DegU. Activation of a DegU regulated promoter occurs via binding of DegU to the promoter (which leads to increased expression of the gene operably linked thereto). Whether a promoter is bound and upregulated (i.e. increased) by DegU can be assessed as described in Ogura et al. (Ogura, M., Shimane, K., Asai, K., Ogasawara, N., & Tanaka, T.; 2003; In: Molecular microbiology, 49(6), 1685-1697). The term increased has been defined herein above.

The DegS-DegU two-component regulatory system of Bacilli controls various cellular differentiation processes in particular during the transition from the exponential to the stationary growth phase and the effect of high levels of DegU-P levels on the enhanced transcription of over 100 genes described (Mäder U, Homuth G., Mol Genet Genomics. 2002 December; 268(4):455-67.) The 5′ gene regulatory regions of those genes comprising transcription factor binding sites e.g. DegU-P, core-promoter, 5′-UTR and Shine Dalgarno sequences are in the scope of the present invention.

Preferred gene regulatory regions are selected but not limited from the genes encoding for extracellular enzymes such as AprE, Bpr, SacB, or synthesis of poly-gamma-glutamate (ywsC (pgsB), ywtA (pgsC), ywtB (pgsA), ywtC (pgsE)). Most preferably, the gene regulatory regions comprise DegU-P binding sites.

The promoter of the bpr gene from Bacillus subtilis with its core promoter region and the DegU binding sites are well described. The transcriptional start site (TSS) is at nucleotide nt −84 relative to the start ATG of the bpr gene. The core promoter encompasses nucleotides nt −1 to nt −38 relative to the TSS and the three DegU binding sites reside within nt −66 to nt −154. More preferably the functional bpr promoter fragment comprises nucleotides nt −1 to nt −175 relative to the TSS (Tsukahara K, Ogura M. FEMS Microbiol Lett. 2008 March; 280(1):8-13).

Likewise, the transcriptional activation of the poly-gamma glutamate synthesis genes by high levels of DegU-P has been described (Ohsawa T, Ogura M. Biosci Biotechnol Biochem. 2009 September; 73(9):2096-102) defining the regions of DegU-P binding to nucleotides nt −24 to nt −47 relative to the transcriptional start site.

The DegU-P binding site with the 5′-regulatory region of the levansucrase gene sacB has been mapped and the stimulatory effect with a degU32(Hy) strain background on the sacB promoter compared to the refence strain shown (Henner D J, Hoch J A., J Bacteriol. 1988 January; 170(1):296-300; Shimotsu H, Henner D J., J Bacteriol. 1986 October; 168(1)).

The native promoter from the gene encoding the Bacillus subtilisin Carlsberg protease, also referred to as aprE promoter, is well described in the art. The aprE gene is transcribed by sigma factor A (sigA) and its expression is highly controlled by several regulators-DegU acting as activator of aprE expression, whereas AbrB, ScoC (hpr) and SinR are repressors of aprE expression (Ferrari, E J. A. Hoch. 1988; J Bacteriol 170: 289-295; Henner, D. J., J. A. Hoch. 1988; J. Bacteriol. 170: 296-300; Park, S. S., R. H. Doi. 1989; J Bacteriol 171: 2657-2665; Gaur, N. K., I. Smith. 1991; J Bacteriol 173: 678-686; Kallio, P. T., M. A. Strauch. 1991; Journal of Biological Chemistry 266: 13411-13417).

The core promoter region comprising the sigma factor A binding sites −35 and −10 have been mapped to the region nt −1-nt −45 relative to the transcriptional start site (Park, S. S., R. H. Doi. 1989; J Bacteriol 171: 2657-2665).

WO0151643 describes the increase of expression by mutating the −35 site of the wild type aprE promoter from TACTAA to the canonical TTGACA-35 site motif (Helmann, J. D. 1995; Nucleic Acids Res. 23: 2351-2360).

The transcriptional start site (TSS) is located at nt −58 relative to the start GTG of the aprE gene. The 5′UTR comprises the ribosome binding site (Shine Dalgarno) and a sequence within nt −58-nt −33 relative to the start GTG forming a very stable stem-loop structure of the 5′-end of the mRNA being responsible for high mRNA transcript stability of up to 25 min (Hambraeus, et al., 2000, Microbiology. 146 Pt 12:3051-3059; Hambraeus et al., 2002, Microbiology. 148(Pt 6):1795-1803).

The region of nt −141-nt −161 relative to the transcriptional start site has been shown to be responsible for full induction in a DegU (SacU) and DegQ (SacQ) dependent manner, whereas regions 5′ of nt −200 up to nt −600 are negatively regulated by ScoC (Hpr) (Henner, D. J., J. A. Hoch. 1988; J. Bacteriol. 170: 296-300). More in depth analysis of DegU-P binding sequences revealed additional regions between nt −70 to −nt −27 relative to the TSS (Shimane K, Ogura M.; J Biochem. 2004 September; 136(3):387-97).

The binding site of the repressing transition state regulator ArbB has been mapped to nt −58 to +nt 15 relative to the transcriptional start site (Strauch, M. A., J. A. Hoch. 1989; EMBO J 8: 1615-1621).

The binding sites of the repressor SinR have been mapped to nt −233 to nt −268 relative to the transcriptional start site (Gaur, N. K., I. Smith. 1991; J Bacteriol 173: 678-686).

Jacobs et al (Jacobs M, Flock J I. 1985; Nucleic Acids Res 13: 8913-8926; Jacobs, M. F. 1995. Expression of the subtilisin Carlsberg-encoding gene in Bacillus licheniformis and Bacillus subtilis. Gene 152: 69-74) discloses the sequence of the aprE (subC) gene and its 5′ region of the Bacillus licheniformis NCIB6816 strain (GenBank accession No. X03341). The regulation of the expression of the subtilisin Carlsberg aprE (subC) gene and the DNA sequences involved are described. The transcriptional start site (TSS) is located at nt −73 and accordingly the 5′ UTR comprising nt −73 to nt −1 relative to the start ATG. The ribosome binding site (Shine Dalgarno) is located at position nt −16 to nt −9. The recognition sequence-10-site (TATAAT-box) of the sigma factor A is highly conserved and located at nt −84 to nt −79 whereas the −35 site (TACCAT) located 17 nt upstream of the −10 site is less conserved compared to standard sigma factor A dependent promoters in Bacillus (Helmann et al., 1995, Nucleic Acids Res. 23: 2351-2360). Promoter truncations from the 5′ end comprising nt −122 to nt −1 and nt −181 to nt −1 (mutant 771 and mutant 770, respectively, as described in Jacobs et al., 1995) show 20-40 fold reduced subtilisin Carlsberg protease expression activities compared to expression with promoter fragment nt −225 to nt −1 (mutant 769, as described in Jacobs et al., 1995) in Bacillus subtilis strains with elevated regulators DegU (degU32H) or DegQ (degQ36H). Therefore, the binding sites of the regulator degU stimulating subtilisin Carlsberg expression lie within the region comprising nt −225 to nt −182.

WO9102792 discloses the functionality of the promoter of the alkaline protease gene for the large-scale production of subtilisin Carlsberg-type protease in Bacillus licheniformis and its production in a fermentation process.

The promoters of the Bacillus pumilus genes aprE1 and aprE2 encoding for Subtilisin proteases have been applied for the expression of recombinant protease and amylase in Bacillus pumilus (Küppers T, Wiechert W. Microb Cell Fact. 2014 Mar. 24; 13(1):46). In particular the PaprE1-III promoter variant comprising nucleotides nt −382 relative to the start ATG showed very high productivity compared to PaprE1-IV promoter variant (nt −357 relative to the start ATG). An “aprE promoter”, “aprE-type promoter” or “aprE promoter sequence” is the nucleotide sequence (or parts or variants thereof) located upstream of an aprE gene, i.e., a gene coding for a Bacillus subtilisin Carlsberg protease, on the same strand as the aprE gene that enables that aprE gene's transcription. The term “transcription start site” or “transcriptional start site” shall be understood as the location where the transcription starts at the 5′ end of a gene sequence. In prokaryotes the first nucleotide, referred to as +1 is in general an adenosine (A) or guanosine (G) nucleotide. In this context, the terms “sites” and “signal” can be used interchangeably herein.

According to the present invention the promoter preferably comprises a degU binding motif. DegU is a transcription factor known to increase expression of aprE-type promoters. Thus, the presence of a corresponding transcription factor binding site is preferred.

Optionally the promoter further comprises one more binding motifs of regulatory factors selected from ScoC (hpr), SinR and AbrB. These regulatory factors are primarily negative regulators.

However, presence of such binding sites is preferred to improve correct timing of expression of the target gene during an industrial fermentation process.

Further optionally the promoter comprises a 5′UTR. This is a transcribed but not translated region downstream of the −1 promoter position. Such untranslated region for example should contain a ribosome binding site to facilitate translation in those cases where the target gene codes for a peptide or polypeptide.

In a particularly preferred embodiment, the DegU regulated promoter is an aprE promoter.

An “aprE promoter” or “aprE promoter sequence” is the nucleotide sequence (or parts or variants thereof) located upstream of an aprE gene, i.e., a gene coding for a Bacillus Subtilisin Carlsberg protease, on the same strand as the aprE gene that enables that aprE gene's transcription.

In a preferred embodiment, the aprE promoter comprises a sequence as shown in SEQ ID NO: 187, 188, 189, 190, 191, 192, 193, 194, 202 or 203, in particular SEQ ID NO: 188, 192 or 194.

In the studies underlying the present invention, a promoter having a sequence as shown in SEQ ID NO: 188 was used for expressing a polynucleotide of interest.

The native promoter from the gene encoding the Carlsberg protease, also referred to as aprE promoter, is well described in the art. The aprE gene is transcribed by sigma factor A (sigA) and its expression is highly controlled by several regulators-DegU acting as activator of aprE expression, whereas AbrB, ScoC (hpr) and SinR are repressors of aprE expression.

WO9102792 discloses the functionality of the promoter of the alkaline protease gene for the large-scale production of subtilisin Carlsberg-type protease in Bacillus licheniformis. In particular, WO9102792 describes the 5′ region of the subtilisin Carlsberg protease encoding aprE gene of Bacillus licheniformis (FIG. 27) comprising the functional aprE gene promoter and the 5′UTR comprising the ribosome binding site (Shine Dalgarno sequence).

The term “transcription start site” or “transcriptional start site” shall be understood as the location where the transcription starts at the 5′ end of a gene sequence. In prokaryotes the first nucleotide, referred to as ±1 is in general an adenosine (A) or guanosine (G) nucleotide. In this context, the terms “sites” and “signal” can be used interchangeably herein. The term “expression” or “gene expression” means the transcription of a specific gene or specific genes or specific nucleic acid construct. The term “expression” or “gene expression” in particular means the transcription of a gene or genes or genetic construct into structural RNA (e.g., rRNA, tRNA) or mRNA with or without subsequent translation of the latter into a protein. The process includes transcription of DNA and processing of the resulting mRNA product. Further optionally the promoter comprises a 5′UTR. This is a transcribed but not translated region downstream of the −1 promoter position. Such untranslated region for example should contain a ribosome binding site to facilitate translation in those cases where the target gene codes for a peptide or polypeptide.

With respect to the 5′UTR the invention in particular teaches to combine the promoter of the present invention with a 5′UTR comprising one or more stabilizing elements. This way the mRNAs synthesized from the promoter region may be processed to generate mRNA transcript with a stabilizer sequence at the 5′ end of the transcript. Preferably such a stabilizer sequence at the 5′end of the mRNA transcripts increases their half-life as described by Hue et al, 1995, Journal of Bacteriology 177: 3465-3471. Suitable mRNA stabilizing elements are those described in
WO08148575, preferably SEQ ID NO. 1 to 5 of WO08140615, or fragments of these sequences which maintain the mRNA stabilizing function, and in
WO08140615, preferably Bacillus thuringiensis CryIIIA mRNA stabilising sequence or bacteriophage SP82 mRNA stabilising sequence, more preferably a mRNA stabilising sequence according to SEQ ID NO. 4 or 5 of WO08140615, more preferably a modified mRNA stabilising sequence according to SEQ ID NO. 6 of WO08140615, or fragments of these sequences which maintain the mRNA stabilizing function.

Preferred mRNA stabilizing elements are selected from the group consisting of aprE, grpE, cotG, SP82, RSBgsiB, CryIIIA mRNA stabilizing elements, or according to fragments of these sequences which maintain the mRNA stabilizing function. A preferred mRNA stabilizing element is the grpE mRNA stabilizing element (corresponding to SEQ ID NO. 2 of WO08148575). The 5′UTR also preferably comprises a modified rib leader sequence located downstream of the promoter and upstream of a ribosome binding site (RBS). In the context of the present invention a rib leader is herewith defined as the leader sequence upstream of the riboflavin biosynthetic genes (rib operon) in a Bacillus cell, more preferably in a Bacillus subtilis cell. In Bacillus subtilis, the rib operon, comprising the genes involved in riboflavin biosynthesis, include ribG (ribD), ribB (ribE), ribA, and ribH genes. Transcription of the riboflavin operon from the rib promoter (Prib) in B. subtilis is controlled by a riboswitch involving an untranslated regulatory leader region (the rib leader) of almost 300 nucleotides located in the 5′-region of the rib operon between the transcription start and the translation start codon of the first gene in the operon, ribG. Suitable rib leader sequences are described in WO2015/1181296, in particular pages 23-25, incorporated herein by reference.

The degQ gene may be expressed under control of any promoter deemed appropriate, such as a constitutive promoter. In an embodiment, the promoter comprises a sequence as shown in SEQ ID NO: 58. Alternatively, the gene is expressed under control of the PaprE promoter (having a sequence as shown in SEQ ID 188) the Pveg promoter or the PspoVG promoter.

In accordance with the present invention, the bacterial host cell shall comprise at least two mutations. The mutations shall modify (i.e. increase or decrease) the activity and/or amount of gene products, such as of polypeptides, or variants thereof, as described elsewhere herein. The term “variant”

Variants of a parent polypeptide may have an amino acid sequence which is at least n percent identical to the amino acid sequence of the respective parent polypeptide with n being an integer between 50 and 100, preferably 50, 55, 60, 65, 70, 75, 80, 85, 90, 91, 92, 93, 94, 95, 96, 97, 98 or 99 compared to the full length polypeptide sequence. Variant enzymes described herein which are n percent identical when compared to a parent enzyme have enzymatic activity. In some embodiments, a variant of a parent polypeptide comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, at least 99%, but below 100% identical to an amino acid sequence of the parent polypeptide.

Variants may be, thus, defined by their sequence identity when compared to a parent polypeptide. Sequence identity usually is provided as “% sequence identity” or “% identity”. To determine the percent-identity between two amino acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gapopen=10.0, gapextend=0.5 and matrix=EBLOSUM62). The preferred alignment for the purpose of this invention is that alignment, from which the highest sequence identity can be determined.

After aligning the two sequences, in a second step, an identity value shall be determined from the alignment. Therefore, according to the present invention the following calculation of percent-identity applies:

%-identity=(identical residues/length of the alignment region which is showing the respective sequence of this invention over its complete length)*100. Thus, sequence identity in relation to comparison of two amino acid sequences according to this embodiment is calculated by dividing the number of identical residues by the length of the alignment region which is showing the respective sequence of this invention over its complete length. This value is multiplied with 100 to give “%-identity”.

For calculating the percent identity of two DNA sequences the same applies as for the calculation of percent identity of two amino acid sequences with some specifications. For DNA sequences encoding for a protein the pairwise alignment shall be made over the complete length of the coding region from start to stop codon excluding introns. For non-protein-coding DNA sequences the pairwise alignment shall be made over the complete length of the sequence of this invention, so the complete sequence of this invention is compared to another sequence, or regions out of another sequence. Moreover, the preferred alignment program implementing the Needleman and Wunsch algorithm (J. Mol. Biol. (1979) 48, p. 443-453) is “NEEDLE” (The European Molecular Biology Open Software Suite (EMBOSS)) with the programs default parameters (gapopen=10.0, gapextend=0.5 and matrix=EDNAFULL).

In one embodiment, a variant polypeptide comprises 1-30, 1-20, 1-10, or 1-5 amino acid substitutions, preferably, such substitutions are not pertaining to the functional domains of an enzyme. Variants may be defined by their sequence similarity when compared to a parent polypeptide. Sequence similarity usually is provided as “% sequence similarity” or “%-similarity”. For calculating sequence similarity in a first step a sequence alignment has to be generated as described above. In a second step, the percent-similarity has to be calculated, whereas percent sequence similarity takes into account that defined sets of amino acids share similar properties, e.g., by their size, by their hydrophobicity, by their charge, or by other characteristics. Herein, the exchange of one amino acid with a similar amino acid is referred to as “conservative mutation”. Polypeptide variants comprising conservative mutations appear to have a minimal effect on protein folding resulting in certain polypeptide, preferably enzyme, properties being substantially maintained when compared to the polypeptide properties of the parent polypeptide. For determination of %-similarity according to this invention the following applies, which is also in accordance with the BLOSUM62 matrix, which is one of the most used amino acids similarity matrix for database searching and sequence alignments. An amino acid exchange is, typically, defined as similar if the value of the BLOSUM62 substitution matrix for the pair of letters is positive. Table 9 shows conservative exchanges.

TABLE 9

Amino acid A is similar to amino acid S
Amino acid D is similar to amino acids E; N
Amino acid E is similar to amino acids D; K; Q
Amino acid F is similar to amino acids W; Y
Amino acid H is similar to amino acids N; Y
Amino acid I is similar to amino acids L; M; V
Amino acid K is similar to amino acids E; Q; R
Amino acid L is similar to amino acids I; M; V
Amino acid M is similar to amino acids I; L; V
Amino acid N is similar to amino acids D; H; S
Amino acid Q is similar to amino acids E; K; R
Amino acid R is similar to amino acids K; Q
Amino acid S is similar to amino acids A; N; T
Amino acid T is similar to amino acid S
Amino acid V is similar to amino acids I; L; M
Amino acid W is similar to amino acids F; Y
Amino acid Y is similar to amino acids F; H; W.

Conservative amino acid substitutions may occur over the full length of the sequence of a polypeptide sequence of a functional protein such as an enzyme. In one embodiment, such mutations are not pertaining to the functional domains of an enzyme. In another embodiment conservative mutations are not pertaining to the catalytic centers of an enzyme.

Therefore, according to the present invention the following calculation of percent-similarity applies:

%-similarity=[(identical residues+similar residues)/length of the alignment region which is showing the respective sequence of this invention over its complete length]*100. Thus, sequence similarity in relation to comparison of two amino acid sequences herein is calculated by dividing the number of identical residues plus the number of similar residues by the length of the alignment region which is showing the respective sequence of this invention over its complete length. This value is multiplied by 100 to give “%-similarity”.

Especially, variant polypeptide comprising conservative mutations which are at least m percent similar to the respective parent sequences with m being an integer between 50 and 100, preferably 50, 55, 60, 65, 70, 75, 80, 85, 90, 91, 92, 93, 94, 95, 96, 97, 98 or 99 compared to the full length polypeptide sequence, are expected to have essentially unchanged polypeptide properties. In another embodiment conservative mutations are not pertaining to the catalytic centers of an enzyme. In one embodiment, a variant polypeptide comprises 1-30, 1-20, 1-10, or 1-5 conservative amino acid substitutions, preferably, such substitutions are not pertaining to the functional domains of an enzyme.

Likewise, the exchange of one amino acid with a non-similar amino acid is referred to as “nonconservative mutation”. Enzyme variants comprising non-conservative mutations appear to have an effect on protein folding resulting in certain enzyme properties being different when compared to the enzyme properties of the parent enzyme. Hence, an amino acid exchange is defined as non-conservative if the value of the BLOSUM62 substitution matrix for the pair of letters is negative. Table 10 shows non-conservative exchanges.

TABLE 10
Amino acid A is non-similar to amino acids D, E, F, H, I, K, L, M, N, P, Q, R, W, Y
Amino acid C is non-similar to amino acids D, E, F, G, H, I, K, L, M, N, P, Q, R, S, T, V, W, Y
Amino acid D is non-similar to amino acids A, C, F, G, H, I, K, L, M, P, R, T, V, W, Y
Amino acid E is non-similar to amino acids A, C, F, G, I, L, M, P, T, V, W, Y
Amino acid F is non-similar to amino acids A, C, D, E, G, H, K, N, P, Q, R, S, T, V
Amino acid G is non-similar to amino acids C, D, E, F, H, I, K, L, M, P, Q, R, T, V, W, Y
Amino acid H is non-similar to amino acids A, C, D, F, G, I, K, L, M, P, S, T, V, W
Amino acid I is non-similar to amino acids A, C, D, E, G, H, K, N, P, Q, R, S, T, W, Y
Amino acid K is non-similar to amino acids A, C, D, F, G, H, I, L, M, P, T, V, W, Y
Amino acid L is non-similar to amino acids A, C, D, E, G, H, K, N, P, Q, R, S, T, W, Y
Amino acid M is non-similar to amino acids A, C, D, E, G, H, K, N, P, R, S, T, W, Y
Amino acid N is non-similar to amino acids A, C, F, I, L, M, P, V, W, Y
Amino acid P is non-similar to amino acids A, C, D, E, F, G, H, I, K, L, M, N, Q, R, S, T, V, W, Y
Amino acid Q is non-similar to amino acids A, C, F, G, I, L, P, T, V, W, Y
Amino acid R is non-similar to amino acids A, C, D, F, G, I, L, M, P, S, T, V, W, Y
Amino acid S is non-similar to amino acids C, F, H, I, L, M, P, R, V, W, Y
Amino acid T is non-similar to amino acids C, D, E, F, G, H, I, K, L, M, P, Q, R, W, Y
Amino acid V is non-similar to amino acids C, D, E, F, G, H, K, N, P, Q, R, S, W, Y
Amino acid W is non-similar to amino acids A, C, D, E, G, H, I, K, L, M, N, P, Q, R, S, T, V
Amino acid Y is non-similar to amino acids A, C, D, E, G, I, K, L, M, N, P, Q, R, S, T, V

A variant of a parent polypeptide may have the activity or function of the parent polypeptide. Table A herein below provides information on the activity/function of the parent polypeptides that may be modified in accordance with the present invention. Variant enzymes described herein with m percent-similarity when compared to a parent enzyme, thus have enzymatic activity.

### Method for Producing a Polypeptide of Interest

The present invention further contemplates the use of the modified Bacillus host cell of the present invention for producing a polypeptide of interest. For producing the polypeptide of interest, the modified Bacillus host cell shall comprise at least one polynucleotide encoding the polypeptide of interest, wherein said polynucleotide is operably linked to a promoter. Accordingly, the host cell shall comprise an expression cassette for at least one polypeptide of interest. Thus, the present invention relates to a method for producing a polypeptide of interest, comprising
a) providing the modified Bacillus host cell of the invention comprising an expression cassette for a polypeptide of interest.
b) cultivating the host cell under conditions which allow for the expression of the polypeptide of interest, and
c) optionally isolating the polypeptide of interest from the cultivation medium.

The explanations and definitions given herein above in connection with the modified host cell of the present invention apply mutatis mutandis to the method of the present invention. The term “cultivating” as used herein refers to keeping alive and/or propagating the modified host cell comprised in a culture at least for a predetermined time. The term encompasses phases of exponential cell growth at the beginning of growth after inoculation as well as phases of stationary growth. The cultivation conditions shall allow for the expression, i.e. the production, of the polypeptide of interest. Such conditions can be chosen by the skilled person without further ado. Exemplary conditions for the cultivation of the modified host cell are described in Example 1. In an embodiment of the method of the present invention, the cultivation in step b) is carried out as fed batch cultivation.

The method of the present invention, if applied, allows for increasing the expression, i.e. the production, of the at least one polypeptide of interest. Preferably, expression is increased as compared to the expression in an unmodified control cell. In a preferred embodiment, expression of the at least one polypeptide of interest is increased by at least 10%, 20% or by at least 40%, such as by at least 50%, or at least 80% as compared to the expression in the control cell. For example, expression of the at least one polypeptide of interest may be increased by 20% to 100%, such as by 40% to 60%, as compared to the control cell. Further, it is envisaged that the expression is increased by at least 100%, 150%, 200%. 250% or 300%, such as by 200% to 300%. Typically, the expression can be measured by determining the amount of the polypeptide in the host cell and/or in the cultivation medium.

If the modified host cell is a B. licheniformis cell, the polypeptide of interest is advantageously produced with an increased purity (due to the decreased expression of ForD) as compared to an unmodified control cell. The method of the present invention, if applied, thus allows for increasing the purity of the compound of interest. Preferably, the purity regarding a particular contamination is increased by at least 10%, at least 20%, at least 30%, at least 40%, at least 50%, at least 60%, at least 70%, at least 80%, at least 90%, or 100% as compared to the purity of the compound of interest produced by a control cell.

Preferably, the Bacillus licheniformis host cell comprises a reduced expression of Formosin D compared to an unmodified control cell, preferably, wherein the Formosin D comprises with increasing preference an amino acid sequence having at least 80%, at least 90%, at least 95%, at least 99%, or 100% identity to SEQ ID NO: 215. Preferably, the modified Bacillus licheniformis host cell comprises no modification in the endogenous forD gene.

### EXAMPLES

### Materials and Methods

The following examples only serve to illustrate the invention. The numerous possible variations that are obvious to a person skilled in the art also fall within the scope of the invention. Unless otherwise stated the following experiments have been performed by applying standard equipment, methods, chemicals, and biochemicals as used in genetic engineering, molecular biology and fermentative production of chemical compounds by cultivation of microorganisms. See also Sambrook et al. (Sambrook, J. and Russell, D. W. Molecular cloning. A laboratory manual, 3^{rd} ed, Cold Spring Harbor Laboratory Press, Cold Spring Harbor, N Y. 2001).

Electrocompetent Bacillus licheniformis Cells and Electroporation

Transformation of DNA into Bacillus licheniformis (U.S. Pat. No. 5,352,604) is performed via electroporation. Preparation of electrocompetent Bacillus licheniformis cells and transformation of DNA is performed as essentially described by Brigidi et al (Brigidi, P., Mateuzzi, D. (1991). Biotechnol. Techniques 5, 5) with the following modification: Upon transformation of DNA, cells are recovered in 1 ml LBSPG buffer and incubated for 60 min at 37° C. (Vehmaanperä J., 1989, FEMS Microbio. Lett., 61: 165-170) following plating on selective LB-agar plates.

In order to overcome the Bacillus licheniformis specific restriction modification system of Bacillus licheniformis strains DSM641, plasmid DNA is isolated from Ec #098 cells as described below. For transfer into Bacillus licheniformis restrictase knockout strains, plasmid DNA is isolated from E. coli INV110 cells (Life technologies).

### Plasmid Isolation

Plasmid DNA was isolated from Bacillus and E. coli cells by standard molecular biology methods described in (Sambrook, J. and Russell, D. W. Molecular cloning. A laboratory manual, 3rd ed, Cold Spring Harbor Laboratory Press, Cold Spring Harbor, N Y. 2001) or the alkaline lysis method (Birnboim, H. C., Doly, J. (1979). Nucleic Acids Res 7(6): 1513-1523). Bacillus cells were in comparison to E. coli treated with 10 mg/ml lysozyme for 30 min at 37° C. prior to cell lysis.

### Plasmids

Plasmid p689-T2A-lac

The E. coli plasmid p689-T2A-lac comprises the lacZ-alpha gene flanked by BpiI restriction sites, again flanked 5′ by the T1 terminator of the E. coli rrnB gene and 3′ by the TO lambda terminator and was ordered as gene synthesis construct (SEQ ID 027).

pEC194RS—Bacillus Temperature Sensitive Deletion Plasmid

The plasmid pE194 is PCR-amplified with oligonucleotides SEQ ID 009 and SEQ ID 010 with flanking PvuII sites, digested with restriction endonuclease PvuII and ligated into vector pCE1 digested with restriction enzyme SmaI. pCE1 is a pUC 18 derivative, where the BsaI site within the ampicillin resistance gene has been removed by a silent mutation. The ligation mixture was transformed into E. coli DH10B cells (Life technologies). Transformants were spread and incubated overnight at 37° C. on LB-agar plates containing 100 μg/ml ampicillin. Plasmid DNA was isolated from individual clones and analyzed for correctness by restriction digest. The resulting plasmid is named pEC194S.

The type-II-assembly mRFP cassette is PCR-amplified from plasmid pBSd141R (accession number: KY995200) (Radeck, J., Meyer, D., Lautenschlager, N., and Mascher, T. 2017. Bacillus SEVA siblings: A Golden Gate-based toolbox to create personalized integrative vectors for Bacillus subtilis. Sci. Rep. 7: 14134) with oligonucleotides SEQ ID 011 and SEQ ID NO: 12, comprising additional nucleotides for the restriction site BamHI. The PCR fragment and pEC194S were restricted with restriction enzyme BamHI following ligation and transformation into E. coli DH10B cells (Life technologies). Transformants were spread and incubated overnight at 37° C. on LB-agar plates containing 100 μg/ml ampicillin. Plasmid DNA was isolated from individual clones and analyzed for correctness by restriction digest. The resulting plasmid pEC194RS carries the mRFP cassette with the open reading frame opposite to the reading frame of the erythromycin resistance gene.

pDel003—aprE Gene Deletion Plasmid

The gene deletion plasmid for the aprE gene of Bacillus licheniformis was constructed with plasmid pEC194RS and the gene synthesis construct SEQ ID 021 comprising the genomic regions 5′ and 3′ of the aprE gene flanked by BsaI sites compatible to pEC194RS. The type-II-assembly with restriction endonuclease BsaI was performed as described (Radeck et al., 2017; Sci. Rep. 7: 14134) and the reaction mixture subsequently transformed into E. coli DH10B cells (Life technologies). Transformants were spread and incubated overnight at 37° C. on LB-agar plates containing 100 μg/ml ampicillin. Plasmid DNA was isolated from individual clones and analyzed for correctness by restriction digest. The resulting aprE deletion plasmid is named pDel003.

pDel005—sigF Gene Deletion Plasmid

The gene deletion plasmid for the sigF gene (spollAC gene) of Bacillus licheniformis was constructed as described for pDel003, however the gene synthesis construct SEQ ID 024 comprising the genomic regions 5′ and 3′ of the sigF gene flanked by BsaI sites compatible to pEC194RS was used. The resulting sigF deletion plasmid is named pDel005.

pDel006—Restrictase Gene Deletion Plasmid

The gene deletion plasmid for the restrictase gene (SEQ ID 014) of the restriction modification system of Bacillus licheniformis DSM641(SEQ ID 013) was constructed with plasmid pEC194RS and the gene synthesis construct SEQ ID 015 comprising the genomic regions 5′ and 3′ of the restrictase gene flanked by BsaI sites compatible to pEC194RS. The type-II-assembly with restriction endonuclease BsaI was performed as described above and the reaction mixture subsequently transformed into E. coli DH10B cells (Life technologies). Transformants were spread and incubated overnight at 37° C. on LB-agar plates containing 100 μg/ml ampicillin. Plasmid DNA was isolated from individual clones and analyzed for correctness by restriction digest. The resulting restrictase deletion plasmid is named pDel006.

pDel007—Poly-Gamma-Glutamate Synthesis Genes Deletion Plasmid

The deletion plasmid for deletion of the genes involved in poly-gamma-glutamate (pga) production, namely ywsC (pgsB), ywtA (pgsC), ywtB (pgsA), ywtC (pgsE) of Bacillus licheniformis was constructed as described for pDel006, however the gene synthesis construct SEQ ID 018 comprising the genomic regions 5′ and 3′ flanking the ywsC(pgsB), ywtA (pgsC), ywtB (pgsA), ywtC (pgsE) genes flanked by BsaI sites compatible to pEC194RS was used. The resulting pga deletion plasmid is named pDel007.

pMA110—Pga::BLAP Integration Plasmid

The pga::BLAP integration plasmid for integration of the BLAP gene expression cassette into the poly-gamma-glutamate synthesis locus was constructed by a two-step cloning strategy. The BLAP protease expression cassette comprising the PaprE promoter was PCR amplified from pCB56C (U.S. Pat. No. 5,352,604) using oligonucleotides Seq ID 028 and Seq ID NO: 029 and subcloned into p689-T2A-lac in a type-II-assembly reaction with restriction endonuclease BpiI. The resulting plasmid p689-BLAP was sequence verified. The plasmid for exchange of the pga synthesis locus by the BLAP expression cassette was constructed in a second type-II-assembly reaction with restriction endonuclease BsaI with plasmids pEC194RS and p689-BLAP, and 5′ and 3′ homology regions of the pga synthesis locus (Seq ID 001 and Seq ID 02) provided as gene synthesis constructs flanked by BsaI restriction sites compatible with pEC194RS and p689-BLAP to allow for directed cloning. The resulting sequence verified plasmid was named pMA110.

### Plnt010—CAT::Psy-degQ Integration Plasmid

The CAT::Psy-degQ integration plasmid for integration of the Bacillus licheniformis degQ gene expression cassette into the chloramphenicol-acetyltransferase (CAT) locus was constructed by a two-step cloning strategy. The degQ expression cassette comprising a synthetic promoter variant of the Bacillus subtilis Pveg promoter referred to as Psy (SEQ ID 058) and the degQ gene fragment of Bacillus licheniformis (SEQ ID 059) were ordered as gene synthesis fragments with flanking BpiI restriction endonuclease sites and subcloned into p689-T2A-lac in a type-II-assembly reaction with restriction endonuclease BpiI. The resulting plasmid p689-Psy-degQ was sequence verified. The plasmid for exchange of the CAT locus by the degQ expression cassette was constructed in a second type-II-assembly reaction with restriction endonuclease BsaI with plasmids pEC194RS and p689-Psy-degQ, and 5′ and 3′ homology regions of the CAT locus (Seq ID 041 and Seq ID 042) provided as gene synthesis constructs flanked by BsaI restriction sites compatible with pEC194RS and p689-Psy-degQ to allow for directed cloning. The resulting sequence verified plasmid was named plnt010.

Plasmid pJOE8999.1:

Altenbuchner J. 2016. Editing of the Bacillus subtilis genome by the CRISPR-Cas9 system. Appl Environ Microbiol 82:5421-5.

Plasmid pJOE-T2A

To allow for type-II-assembly (T2A) based one-step-cloning of the sgRNA and the homology regions for DSB repair the CRISPR/Cas9 plasmid pJOE8889.1 was modified as follows. The type-II-assembly mRFP cassette from plasmid pBSd141R (accession number: KY995200) (Radeck et al., J 2017; Sci. Rep. 7: 14134) was modified such to remove multiple restriction sites and the BpiI restriction sites and ordered as gene synthesis fragment with flanking SfiI restriction sites (SEQ ID 030). The plasmid is named p #732. Plasmid p #732 and plasmid pJOE8999.1 were digested with SfiI (New England Biolabs, NEB) and the mRFP cassette of p #732 ligated into SfiI-digested pJOE8999.1 following transformation into competent E. coli DH10B cells. Positive clones were screened on IPTG/X-Gal and kanamycin (20 μg/ml) containing LB agar plates for purple colonies (blue-white screening and mRFP1 expression). The resulting sequence-verified plasmid was named pJOE-T2A.

Plasmid pCC027—T2A CRISPR Destination Vector

Plasmid pCC027 is a derivative of the plasmid pJOE-T2A, where the promoter PmanP was replaced by a promoter fragment (SEQ ID 031) comprising in the 5′ to 3′ orientation the terminator region of pMutin2 (accession number AF072806) followed by a Pveg promoter variant derived from Guiziou et al (Guiziou, S., V. Sauveplane, H. J. Chang, C. Clerte, N. Declerck, M. Jules, and J. Bonnet. 2016. A part toolbox to tune genetic expression in Bacillus subtilis. Nucleic Acids Res. 44: 7495-7508) by the Gibson assembly method (NEBuilder® HiFi DNA Assembly Cloning Kit, New England Biolabs).

pDel004—amyB Gene Deletion Plasmid

The gene deletion plasmid for the amyB gene of Bacillus licheniformis was constructed as described for pDel003, however the gene synthesis construct SEQ ID 217 comprising the genomic regions 5′ and 3′ of the amyB gene flanked by BsaI sites compatible to pEC 194RS was used. The resulting amyB deletion plasmid is named pDel004.

pDel034—remA Loss of Function Plasmid

To inactivate RemA, the wildtype allele of Bacillus licheniformis was exchanged by a mutated copy of the remA gene at its native locus, resulting in expression of a RemA with the combined loss of function mutations R18W and P29S (Winkelman, J. TKearns, D. B. (2009): Journal of bacteriology 191 (12), S. 3981-3991). The remA R18W, P29S gene with the 5′ and 3′ flanking regions flanked by BsaI sites compatible to pEC194RS was ordered as gene synthesis construct SEQ ID 218. The gene editing plasmid was constructed as described for pDel003. The resulting remA editing plasmid was named pDel034.

pDel023—Formosin D Deletion Plasmid

The gene deletion plasmid for the formosin D gene (forD gene) of Bacillus licheniformis was constructed as described for pDel003, however the gene synthesis construct SEQ ID NO: 216 comprising the genomic regions 5′ and 3′ of the forD gene with mutated RBS sequence flanked by BsaI sites compatible to pEC194RS was used. The resulting forD deletion plasmid is named pDel023.

### General Procedure for Construction of Target Specific CRISPR/Cas9 Plasmids

To construct target-specific CRISPR/Cas9-based gene deletion plasmids based on plasmid pCC027, a 20 bp spacer sequence was designed. The spacer was assembled by annealing of two complementary oligonucleotides each carrying a 4 bp extension suitable for cloning into BsaI sites of plasmid pCC0027. The homology directed repair (HDR) region was synthesized by SOE-PCR (splicing by overlapping extension PCR) of two PCR fragments comprising the 5′ and 3′ flanking region of the target gene. BsaI restriction endonuclease sites were introduced as overhangs to the 5′ region forward primer and the 3′ region reverse primer. The 5′ and 3′ fragments were column purified prior to SOE-PCR to remove oligonucleotides used in the first reaction. The fused homology directed repair (HDR) template was purified by agarose gel extraction. The oligonucleotide duplex and the HDR template were cloned into pCC027 plasmid in a one-step type-II-assembly reaction. The type-II-assembly reaction mixture was transformed into E. coli DH10B cells (Life technologies). Transformants were spread on LB agar plates containing X-Gal, IPTG and kanamycin (20 μg/ml) and incubated overnight at 37° C. Plasmid DNA was isolated from individual clones, analyzed by restriction digest or PCR and sequence verified.

### Annealing of Oligonucleotides to Form Oligonucleotide-Duplexes

Oligonucleotides were adjusted to a concentration of 100 μM in water. 5 μl of the forward and 5 μl of the corresponding reverse oligonucleotide were added to 90 μl 30 mM HEPES-buffer (pH 7.8). The reaction mixture was heated to 95° C. for 5 min following annealing by ramping from 95° C. to 4° C. with decreasing the temperature by 0.1° C./sec (Cobb, R. E., Wang, Y., & Zhao, H. (2015). High-Efficiency Multiplex Genome Editing of Streptomyces Species Using an Engineered CRISPR/Cas System. ACS Synthetic Biology, 4(6), 723-728).

pCC031—degU32(Hy) Plasmid for Allelic Exchange

Construction of DegU H12L hyperphosphorylation mutant was achieved by exchange of the wildtype gene with the degU32 allele also known as sacU32 (Kunst, F.; Pascal, M.; LepesantKejzlarova, J.; Lepesant, J. A.; Billault, A.; Dedonder, R. (1974): Pleiotropic mutations affecting sporulation conditions and the syntheses of extracellular enzymes in Bacillus subtilis 168. Biochemie 56 (11-12), S. 1481-1489). The construction of the degU32 genome editing construct to introduce the DegU H12L mutation was performed as described above, however, the degU32 homology regions introducing the mutations for the DegU H12L mutation as well as the introduction of a silent point mutation to remove the PAM site were ordered as gene synthesis construct (Geneart, Regensburg) with flanking BsaI sites (SEQ ID 032). The 20 bp target sequence of the degU gene for the sgRNA was designed and the resulting oligonucleotides SEQ ID 033 and SEQ ID 034 with 5′ phosphorylation were annealed to form an oligonucleotide duplex as described above.

pCC050—bslA Gene Deletion Plasmid

The bs/A coding region (accession number AAU42812; locus tag: Bacillus licheniformis DSM13=BLi03999) was cross-checked by identification of a characteristic N-terminal sequence and the C-terminal CxC motif to distinguish the BsIA protein from its paralogue YweA (Morris, R. J.; Schor, M.; Gillespie, R. M. C.; Ferreira, A. S.; Baldauf, L.; Earl, C. et al. (2017): Natural variations in the biofilm-associated protein BsIA from the genus Bacillus. Sci. Rep. 7 (1), S. 6730). To construct the bs/A gene deletion vector, the 5′ and 3′ homologous flanking regions were amplified with oligonucleotides SEQ ID NO: 3, SEQ ID NO: 4, SEQ ID NO: 5, SEQ ID NO: 6 using Bacillus licheniformis gDNA as a template and fused by SOE-PCR as described above. The bs/A specific spacer sequence was constructed by annealing of oligonucleotides Seq ID 007 and Seq ID 008. The oligonucleotide duplex and the HDR template were cloned into pCC027 in a one-step type-II-assembly reaction. The resulting plasmid was named pCC050.

pCC051—epsA-epsO Gene Deletion Plasmid

To delete the epsABCDEFGHIJKLMNO operon (yvek-yvfF) the homologous flanking regions were amplified with oligonucleotides SEQ ID NO: 35 and SEQ ID NO: 36 and SEQ ID NO: 37 and SEQ ID NO: 38 using Bacillus licheniformis gDNA as a template. The 5′ and 3′ fragments were fused by SOE-PCR generating the HDR template. The spacer sequence was constructed by annealing of oligonucleotides SEQ ID NO: 39 and SEQ ID NO: 40. The oligonucleotide duplex and the HDR template were cloned into pJOE-T2A in a one-step type-II-assembly reaction. The resulting plasmid was named pCC051.

pCC052—slrA Gene Deletion Plasmid For deletion of the sirA gene (accession number AAU42855; locus tag: Bacillus licheniformis DSM13=BLi04042, SEQ ID NO: 152) the homologous flanking regions were amplified with oligonucleotides Seq ID 043 and Seq ID 044 and Seq ID 045 and Seq ID 046 using Bacillus licheniformis gDNA as a template. The 5′ nd 3′ fragments were fused by SOE-PCR generating the HDR template. The sirA specific spacer sequence was constructed by annealing of oligonucleotides Seq ID 037 and Seq ID 048. The oligonucleotide duplex and the HDR template were cloned into pJOE-T2A in a one-step type-II-assembly reaction. The resulting plasmid was named pCC052.
pCC053—tapA-sipW-tasA Gene Deletion Plasmid

For deletion of the tapA-sipW-tasA operon the homologous flanking regions were amplified with oligonucleotides Seq ID 049 and Seq ID 050 and Seq ID 051 and Seq ID 052 using gDNA of Bacillus licheniformis as a template. The 5′ and 3′ fragments were fused by SOE-PCR generating the HDR template. The tapA-sipW-tasA specific spacer sequence was constructed by annealing of oligonucleotides SEQ ID 053 and Seq ID 054. The oligonucleotide duplex and the HDR template were cloned into pJOE-T2A in a one-step type-II-assembly reaction. The resulting plasmid was named pCC053.

### Strains

E. coli Strain Ec #098

E. coli strain Ec #098 is an E. coli INV110 strain (Life technologies) carrying the DNAmethyltransferase encoding expression plasmid pMDS003 WO2019016051.

Generation of Bacillus licheniformis Gene k.o. Strains

For gene deletion in Bacillus licheniformis strains (U.S. Pat. No. 5,352,604) and derivatives thereof deletion plasmids were transformed into E. coli strain Ec #098 made competent according to the method of Chung (Chung, C. T., Niemela, S. L., and Miller, R. H. (1989). One-step preparation of competent Escherichia coli: transformation and storage of bacterial cells in the same solution. Proc. Natl. Acad. Sci. U.S.A 86, 2172-2175), following selection on LB-agar plates containing 100 μg/ml ampicillin and 30 μg/ml chloramphenicol at 37° C. Plasmid DNA was isolated from individual clones and used for subsequent transfer into Bacillus licheniformis strains. The isolated plasmid DNA carries the DNA methylation pattern of Bacillus licheniformis strains (U.S. Pat. No. 5,352,604) respectively and is protected from degradation upon transfer into Bacillus licheniformis. For gene deletion in Bacillus licheniformis strains with deleted restrictase gene, deletion plasmids were constructed within E. coli INV110 cells (Life technologies).

Bacillus licheniformis P304: Deleted Restriction Endonuclease

Electrocompetent Bacillus licheniformis cells were prepared as described above and transformed with 1 μg of pDel006 restrictase gene deletion plasmid isolated from E. coli Ec #098 following plating on LB-agar plates containing 5 μg/ml erythromycin at 30° C.

The gene deletion procedure was performed as described in the following:

Plasmid carrying Bacillus licheniformis cells were grown on LB-agar plates with 5 μg/ml erythromycin at 45° C. driving integration of the deletion plasmid via Campbell recombination into the chromosome with one of the homology regions of pDel006 homologous to the sequences 5′ or 3′ of the restrictase gene. Clones were picked and cultivated in LB-media without selection pressure at 45° C. for 6 hours, following plating on LB-agar plates with 5 μg/ml erythromycin and incubation overnight at 30° C. Individual clones were picked and screened by colony-PCR analysis with oligonucleotides SEQ ID 016 and SEQ ID 017 for successful genomic deletion of the restrictase gene. Putative deletion positive individual clones were picked and taken through two consecutive overnight incubations in LB media without antibiotics at 45° C. to cure the plasmid and plated on LB-agar plates for overnight incubation at 37° C. Single clones were analyzed by colony PCR for successful genomic deletion of the restrictase gene. A single erythromycin-sensitive clone with the correct deleted restrictase gene was isolated and designated Bacillus licheniformis P304.

Bacillus licheniformis P305: deleted sigF gene

Electrocompetent Bacillus licheniformis P304 cells were prepared as described above and transformed with 1 μg of pDel005 sigF gene deletion plasmid isolated from E. coli INV110 cells (Life technologies) following plating on LB-agar plates containing 5 μg/ml erythromycin and incubation overnight at 30° C.

The gene deletion procedure was performed as described for the restrictase gene.

The deletion of the sigF gene was analyzed by PCR with oligonucleotides SEQ ID 025 and SEQ ID 026. The resulting Bacillus licheniformis strain with a deleted sigF gene is designated Bacillus licheniformis P305 and is no longer able to sporulate as described (WO9703185).

Bacillus licheniformis P307: Deleted aprE Gene

Electrocompetent Bacillus licheniformis P305 cells were prepared as described above and transformed with 1 μg of pDel003 aprE gene deletion plasmid isolated from E. coli INV110 cells following plating on LB-agar plates containing 5 μg/ml erythromycin and incubation overnight at 30° C.

The gene deletion procedure was performed as described for the deletion of the restrictase gene. The deletion of the aprE gene was analyzed by PCR with oligonucleotides SEQ ID 022 and SEQ ID 023 The resulting Bacillus licheniformis strain with deleted aprE gene was named Bacillus licheniformis P307.

Bacillus licheniformis M309: Deleted Poly-Gamma Glutamate Synthesis Genes

Electrocompetent Bacillus licheniformis P307 cells were prepared as described above and transformed with 1 μg of pDel007 pga gene deletion plasmid isolated from E. coli INV110 cells (Life technologies) following plating on LB-agar plates containing 5 μg/ml erythromycin and incubation overnight at 30° C.

The gene deletion procedure was performed as described for the deletion of the restrictase gene. The deletion of the pga genes was analyzed by PCR with oligonucleotides SEQ ID 019 and SEQ ID 020 The resulting Bacillus licheniformis strain with deleted pga synthesis genes was named Bacillus licheniformis M309.

Bacillus licheniformis M409: Integration of the BLAP Protease Expression Cassette into the Poly-Gamma Glutamate Synthesis (Pga) Locus

Electrocompetent Bacillus licheniformis P307 cells were prepared as described above and transformed with 1 μg of pMA110-pga::PaprE-BLAP gene deletion/integration plasmid isolated from E. coli INV110 cells (Life technologies) following plating on LB-agar plates containing 5 μg/ml erythromycin and incubation overnight at 30° C.

The gene deletion/integration procedure was performed as described for the deletion of the restrictase gene. The integration of the BLAP expression cassette by replacement of the pga genes was analyzed by PCR with oligonucleotides SEQ ID NO: 19 and SEQ ID NO: 20. The resulting Bacillus licheniformis strain with integrated BLAP expression cassette gene was named Bacillus licheniformis M409.

Bacillus licheniformis PC30: Deleted tapA-sipW-tasA Operon

The deletion of the tapA-sipW-tasA operon was performed as follows. Electrocompetent Bacillus licheniformis M409 cells were prepared as described above and transformed with 1 μg of pCC053 plasmid isolated from E. coli INV110 cells following plating on LB-agar plates containing 20 μg/ml kanamycin and incubation overnight at 37° C.

The next day clones of the transformation reaction were subjected to colony PCR to analyze for successful deletion of the tapA-sipW-tasA operon of Bacillus licheniformis. Positive clones were transferred onto fresh LB-agar plates without antibiotics following incubation at 48° C. overnight for plasmid curing. Kanamycin sensitive clones were again analyzed by PCR and the deleted gene locus was sequence verified. The resulting strain was named Bacillus licheniformis PC30.

Bacillus licheniformis PC31: Deleted epsA-0 Operon

Bacillus licheniformis strain PC31 carrying the epsA-O operon deletion was constructed as described for Bacillus licheniformis strain PC30, however plasmid pCC051 was used.

Bacillus licheniformis PC40: Deleted epsA-0 Operon and Deleted tapA-sipW-tasA Operon

The epsA-O operon deletion was introduced into Bacillus licheniformis strain PC31 carrying the tapA-sipW-tasA operon deletion as described for Bacillus licheniformis strain PC31 using plasmid pCC051

Bacillus licheniformis PC54: Deleted bslA Gene

Bacillus licheniformis strain PC54 carrying the bs/A (yuaB) deletion was constructed as described for Bacillus licheniformis strain PC30, however plasmid pCC050 was used.

Bacillus licheniformis PC32: Deleted slrA Gene

Bacillus licheniformis strain PC32 carrying the slrA deletion was constructed as described for Bacillus licheniformis strain PC30, however plasmid pCC052 was used.

Bacillus licheniformis PC36: degU H12L Mutation

Bacillus licheniformis strain PC36 carrying the mutated degU32 allele resulting in the DegU H12L mutation was constructed as described for Bacillus licheniformis strain PC30, however plasmid pCC031 was used.

Bacillus licheniformis PC38: Deleted epsA-O Operon and degU H12L Mutation

The degU32 (DegU H12L) mutation was introduced into Bacillus licheniformis strain PC31 (ΔepsA-O) as described for Bacillus licheniformis strain PC36 using plasmid pCC031.

Bacillus licheniformis PC39: Deleted tapA-sipW-tasA Operon and degU H12L Mutation

The degU32 (DegU H12L) mutation was introduced into Bacillus licheniformis strain PC30 (ΔtapA-sipW-tasA) as described for Bacillus licheniformis strain PC36 using plasmid pCC031.

Bacillus licheniformis PC41: Deleted epsA-O and tapA-sipW-tasA Operon Combined with the degU H12L Mutation

The degU32 (DegU H12L) mutation was introduced into Bacillus licheniformis strain PC40 (ΔepsA-O, ΔtapA-sipW-tasA) as described for Bacillus licheniformis strain PC36 using plasmid pCC031.

Bacillus licheniformis PC55: Deleted bslA and degU H12L Mutation

The degU32 (DegU H12L) mutation was introduced into Bacillus licheniformis strain PC54 (ΔbslA) as described for Bacillus licheniformis strain PC36 using plasmid pCC031.

Bacillus licheniformis PC56: Combined Deletion of epsA-O, tapA-sipW-tasA, bslA and Introduction of the degU H12L Mutation

Bacillus licheniformis strain PC56 carrying the epsA-O, tapA-sipW-tasA, bslA (yuaB) deletion and the degU32 (DegU H12L) allele was constructed as described for Bacillus licheniformis strain PC54 using plasmid pCC0050 and Bacillus licheniformis PC41 as the parental strain.

Bacillus licheniformis PC51: Deleted slrA and degU H12L Mutation

The degU32 (DegU H12L) mutation was introduced into Bacillus licheniformis strain PC32 (ΔslrA) as described for Bacillus licheniformis strain PC36 using plasmid pCC031.

Bacillus licheniformis PC57: Integration of the degQ Expression Cassette into the CAT Locus

Electrocompetent Bacillus licheniformis PM409 cells were prepared as described above and transformed with 1 μg of plnt010 gene deletion/integration plasmid isolated from E. coli INV110 cells (Life technologies) following plating on LB-agar plates containing 5 μg/ml erythromycin and incubation overnight at 30° C.

The gene deletion/integration procedure was performed as described for the deletion of the restrictase gene. The integration of the degQ expression cassette by replacement of the CAT-locus was analyzed by PCR. The resulting Bacillus licheniformis strain with integrated degQ expression cassette gene was named Bacillus licheniformis PC57.

Bacillus licheniformis PC58: Integration of the degQ Expression Cassette into the PC40 Strain

The gene deletion/integration procedure with plasmid plnt010 was performed in Bacillus licheniformis PC40 as described for the construction of Bacillus licheniformis PC57. The resulting Bacillus licheniformis strain with integrated degQ expression cassette gene was named Bacillus licheniformis PC58.

aprE Gene Deletion Strain Bli #002

Electrocompetent Bacillus licheniformis cells as described in U.S. Pat. No. 5,352,604 were prepared as described above and transformed with 1 μg of pDel003 aprE gene deletion plasmid isolated from E. coli Ec #098 following plating on LB-agar plates containing 5 μg/ml erythromycin at 30° C. The gene deletion procedure was performed as described in the following:

Plasmid carrying Bacillus licheniformis cells were grown on LB-agar plates with 5 μg/ml erythromycin at 45° C. forcing integration of the deletion plasmid via Campbell recombination into the chromosome with one of the homology regions of pDel003 homologous to the sequences 5′ or 3′ of the aprE gene. Clones were picked and cultivated in LB-media without selection pressure at 45° C. for 6 hours, following plating on LB-agar plates with 5 μg/ml erythromycin at 30° C. Individual clones were picked and analyzed by colony-PCR for successful deletion of the aprE gene. Putative deletion positive individual clones were picked and taken through two consecutive overnight incubation in LB media without antibiotics at 45° C. to cure the plasmid and plated on LB-agar plates for overnight incubation at 30° C. Single clones were again restreaked on LB-agar plates with 5 μg/ml erythromycin and analyzed by colony PCR for successful deletion of the aprE gene. A single erythromycin-sensitive clone with the correct deleted aprE gene was isolated and designated Bli #002

amyB Gene Deletion Strain Bli #003

Electrocompetent Bacillus licheniformis Bli #002 cells were prepared as described above and transformed with 1 μg of pDel004 amyB gene deletion plasmid isolated from E. coli Ec #098 following plating on LB-agar plates containing 5 μg/ml erythromycin at 30° C.

The gene deletion procedure was performed as described for the aprE gene.

The deletion of the amyB gene was analyzed by PCR. The resulting Bacillus licheniformis strain with a deleted aprE and deleted amyB gene is designated Bli #003.

sigF Gene Deletion Strain Bli #004

Electrocompetent Bacillus licheniformis Bli #003 cells were prepared as described above and transformed with 1 μg of pDel005 sigF gene deletion plasmid isolated from E. coli Ec #098 following plating on LB-agar plates containing 5 μg/ml erythromycin at 30° C.

The gene deletion procedure was performed as described for the aprE gene.

The deletion of the sigF gene was analyzed by PCR. The resulting Bacillus licheniformis strain with a deleted aprE, a deleted amyB gene and a deleted sigF gene is designated Bli #004. Bacillus licheniformis strain Bli #004 is no longer able to sporulate as described (WO9703185).

### Poly-Gamma Glutamate Synthesis Genes Deletion Strain Bli #008

Electrocompetent Bacillus licheniformis Bli #004 cells were prepared as described above and transformed with 1 μg of pDel007 pga gene deletion plasmid isolated from E. coli Ec #098 following plating on LB-agar plates containing 5 μg/ml erythromycin at 30° C.

The gene deletion procedure was performed as described for the deletion of the aprE gene. The deletion of the pga genes was analyzed by PCR. The resulting Bacillus licheniformis strain with a deleted aprE, a deleted amyB gene, a deleted sigF gene and a deleted pga gene cluster is designated Bli #008.

remA R18W P29S Strain Bli #030

Electrocompetent Bacillus licheniformis Bli #008 cells were prepared as described above and transformed with 1 μg of pDel034 remA gene editing plasmid isolated from E. coli Ec #098 following plating on LB-agar plates containing 5 μg/ml erythromycin at 30° C.

The gene deletion procedure was performed as described for the deletion of the aprE gene. The gene editing of the remA gene was analyzed by PCR with oligonucleotides following restriction enzyme cleavage with ClaI restriction endonuclease. The resulting Bacillus licheniformis strain with a deleted aprE, a deleted amyB gene, a deleted sigF gene, deleted pga gene cluster and mutated remA R18W P19S is designated Bli #030.

Bacillus licheniformis P311: Deleted forD Gene

Electrocompetent Bacillus licheniformis M309 cells were prepared as described above and transformed with 1 μg of pDel023 forD gene deletion plasmid isolated from E. coli INV110 cells following plating on LB-agar plates containing 5 μg/ml erythromycin and incubation overnight at 30° C.

The gene deletion procedure was performed as described for the deletion of the restrictase gene. The deletion of the forD gene was analyzed by PCR with oligonucleotides. The resulting Bacillus licheniformis strain with deleted forD gene was named Bacillus licheniformis P311.

Bacillus licheniformis P312: Integration of the degQ Expression Cassette into the CAT Locus

Electrocompetent Bacillus licheniformis M309 cells were prepared as described above and transformed with 1 μg of plnt010 gene deletion/integration plasmid isolated from E. coli INV110 cells (Life technologies) following plating on LB-agar plates containing 5 μg/ml erythromycin and incubation overnight at 30° C.

The gene deletion/integration procedure was performed as described for the deletion of the restrictase gene. The integration of the degQ expression cassette by replacement of the CAT-locus was analyzed by PCR. The resulting Bacillus licheniformis strain with integrated degQ expression cassette gene was named Bacillus licheniformis P312.

Bacillus licheniformis P313: Deleted forD Gene

Electrocompetent Bacillus licheniformis M409 cells were prepared as described above and transformed with 1 μg of pDel023 forD gene deletion plasmid isolated from E. coli INV110 cells following plating on LB-agar plates containing 5 μg/ml erythromycin and incubation overnight at 30° C.

The gene deletion procedure was performed as described for the deletion of the restrictase gene. The deletion of the forD gene was analyzed by PCR with oligonucleotides. The resulting Bacillus licheniformis strain with deleted forD gene was named Bacillus licheniformis P313

degQ Promoter Analysis

The domestic Bacillus subtilis strain 168 contains a nucleotide exchange within the promoter region of the degQ gene that results in lower degQ expression levels, whereas the degQ36Hy mutation resembles the reverse mutation to wild-type promoter sequence (Stanley N R, Lazazzera B A. Mol Microbiol. 2005; 57(4):1143-1158). Accordingly, the promoters within the 5′ region of the degQ gene from different domesticated and undomesticated Bacillus subtilis strains as well as other Bacillus species were analyzed for the nucleotide exchange within the conserved promoter regions.

The promoter regions were obtained and aligned as follows: The full genome assemblies from various Bacillus strains were manually collected from NCBI Genomes, whereby only completed genomes were considered for further analysis. Type strains and reference genomes were obtained whenever available. The degQ gene was identified primarily by name as annotated in the genome. For two strains, namely for Bacillus pumilus SH-B9 and Bacillus velezensis FZB42, the degQ gene was identified as the top BLAST search result (by bitscore) using the Bacillus licheniformis DSM13 DegQ protein as query (SEQ ID 170). The BLAST search was performed using Geneious Prime 2020.1.2 with the program tblastn and the following parameters: BLOSUM80 matrix, max evalue 0.1, gap open cost 10, gap extend cost 1, low complexity filter enabled, maximum 10 hits. ‘For promoter alignment purposes, promoters were defined as sequences upstream of the degQ 5′ end, extending to the start/end of the upstream annotated gene following manual sequence extraction from the degQ gene strand.

Full promoter regions were aligned with Geneious Prime 2020.1.2, with the following parameters:
aligner: ClustalO version 1.2.2
Number of refinement iterations (—iter): 30
Using full distance matrix for the initial and iterative guide tree calculation (—full, —full-iter)

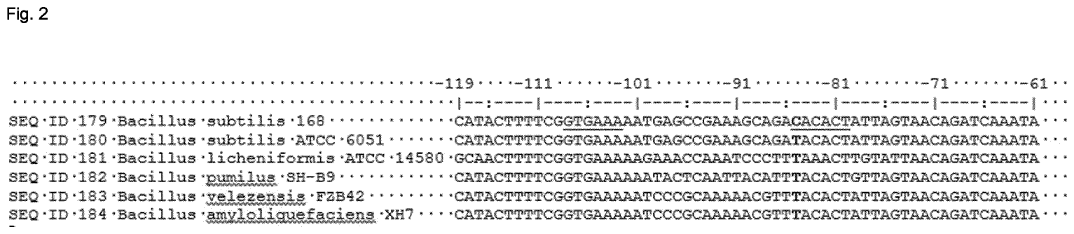

The final alignment was truncated to nucleotides −61 to −119 relative to the translation start codon to allow readability as depicted in FIG. 2.

Table 1 summarizes the information for the degQ promoter analysis.

TABLE 1

| | DegQ PRT | degQ-5′region |
|---|---|---|
| Organism | [SEQ ID] | [SEQ ID] |
| Bacillus subtilis 168 | 160 | 179 |
| Bacillus subtilis strain NCIB | 160 | 180 |
| 3610 | | |
| Bacillus licheniformis strain | 170 | 181 |
| ATCC 14580 = DSM 13 | | |
| Bacillus pumilus strain SH-B9 | 176 | 182 |
| Bacillus velezensis FZB42 | 177 | 183 |
| Bacillus amyloliquefaciens XH7 | 178 | 184 |

### Example 1

Bacillus licheniformis strains with genetic modifications (as indicated in Table 2) that lead to decreased production of components of biofilm and increased levels of phosphorylated DegU were cultivated in a microtiter plate-based fed-batch process (Habicher et al., 2019 Biotechnol J.; 15(2)).

TABLE 2
Bacillus licheniformis mutant strains

| Bacillus licheniformis | |
|---|---|
| strain name | Genotype |
| DSM641 (US5352604) | Wild type |
| P304 | DSM641 DRe |
| P305 | DSM641 DRe, DsigF |
| P307 | DSM641 DRe, DsigF, DaprE |
| M309 | DSM641 DRe, DaprE, DsigF, Dpga |
| M409 | DSM641 DRe, DaprE, DsigF, pga::BLAPR |
| PC30 | M409 DtapA-sipW-tasA |
| PC31 | M409 DepsA-O |
| PC40 | M409 DepsA-O, DtapA-sipW-tasA |
| PC54 | M409 DbslA |
| PC32 | M409 DslrA |
| PC36 | M409 degU32 (H12L) |
| PC39 | M409 DtapA-sipW-tasA, degU32 |
| PC38 | M409 DepsA-O, degU32 |
| PC41 | M409 DepsA-O, DtapA-sipW-tasA, degU32 |
| PC55 | M409 DbslA, degU32 |
| PC56 | M409 DepsA-O, DtapA-sipW-tasA, |
| | degU32, DbslA |
| PC51 | M409 DslrA, degU32 |

‘D’ denotes deleted;
RE: restriction endonuclease

All cultivations were conducted in an orbital shaker with a diameter of 25 mm (Innova 42, New Brunswick Scientific, Eppendorf AG; Hamburg, Germany) at 30° C. and 400 rpm. Strains were cultivated in two subsequent precultures in FlowerPlates (MTP-48-OFF, m2p-labs GmbH) for synchronization of growth. The first preculture was carried out in 800 μl TB medium inoculated with a fresh single colony from the strain streaked onto LB agar plates. After 20 h, the second preculture containing 800 μl V3 minimal medium (Meissner et al., 2015, Journal of industrial microbiology & biotechnology 42 (9): 1203-1215) was inoculated with 8 μl of the first preculture and cultivated for 24 h. Microtiter plate-based fed-batch main cultivations were conducted using 48-well round- and deep-well-microtiter plates with glucose-containing polymer on the bottom of each well (FeedPlate, article number: SMFP08004, Kuhner Shaker GmbH; Herzogenrath, Germany). 70 μl of the second preculture were used to inoculate 700 μl V3 minimal medium without glucose. Main cultures were incubated for 72 h. Precultures were covered with a sterile gas-permeable sealing foil (AeraSeal film, Sigma-Aldrich) to avoid contamination. FeedPlates were sealed with a sterile gas-permeable, evaporation reducing foil (F-GPR48-10, m2p-labs GmbH) to reduce evaporation and to avoid contamination.

At the end of the fermentation process, samples were withdrawn and the protease activity determined photometrically: proteolytic activity was determined by using Succinyl-Ala-Ala-ProPhe-p-nitroanilide (Suc-AAPF-pNA, short AAPF; see e.g. DelMar et al. (1979), Analytical Biochem 99, 316-320) as substrate. pNA was cleaved from the substrate molecule by proteolytic cleavage at 30° C., pH 8.6 TRIS buffer, resulting in release of yellow color of free pNA which was quantified by measuring at OD405.

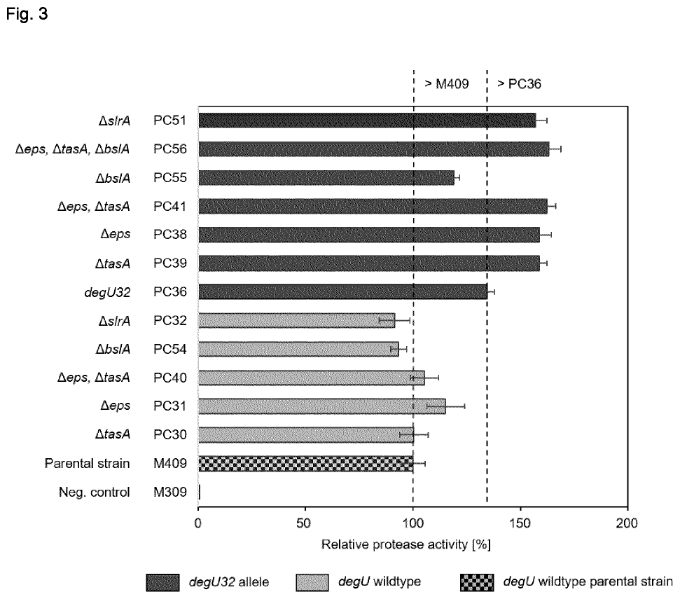

The protease yield of Bacillus licheniformis strain M409 was set to 100% and the protease yield of the other Bacillus licheniformis strains referenced to M409 accordingly. In FIG. 3 the relative protease activity in percent of the mutant Bacillus licheniformis strains for the 72 h timepoints of fed-batch cultivation is plotted in comparison to the Bacillus licheniformis strain M409 and negative control strain M309. Deletion of eps (epsA-O, Strain PC31) improved protease expression as previously described. In contrast, single inactivation of tasA (tapA-sipW-tasA, Strain PC30), bs/A (Strain PC54), and slrA (Strain PC32) as well as inactivation of both eps and tasA (Strain PC40) results in slightly reduced or rather wildtype like protease activity. Allelic exchange of degU by degU32 leads to enhanced protease expression 1.25-fold as previously described. Inactivation of tasA, eps, and slrA in the degU32 strain background (Strains PC39, PC38, PC51) further improved protease expression 1.5-1.6 fold as compared to the parental strain M409 and 1.2-1.3 fold as compared to the degU32 single mutant strain PC36. Combination of inactivated bslA gene and the degU32 allele (Strain PC55) does not improve protease expression although transcription of the biofilm component bs/A is directly activated by DegU-P in Bacillus.

### Example 2

Bacillus licheniformis strains with genetic modifications (as indicated in Table 3) that lead to decreased production of components of biofilm and increased levels of phosphorylated DegU were cultivated in a microtiter plate-based fed-batch process as described for Example 1.

TABLE 3
Bacillus licheniformis mutant strains

| Bacillus licheniformis | |
|---|---|
| strain name | Genotype |
| PC36 | M409 degU32 (H12L) |
| PC57 | M409 CAT::Psy-DegQ |
| PC58 | M409 DepsA-O, DtapA-sipW-tasA, |
| | CAT::Psy-DegQ |

‘D’ denotes deleted;
RE: restriction endonuclease

At the end of the fermentation process, samples were withdrawn and the protease activity determined photometrically as described for Example 1.

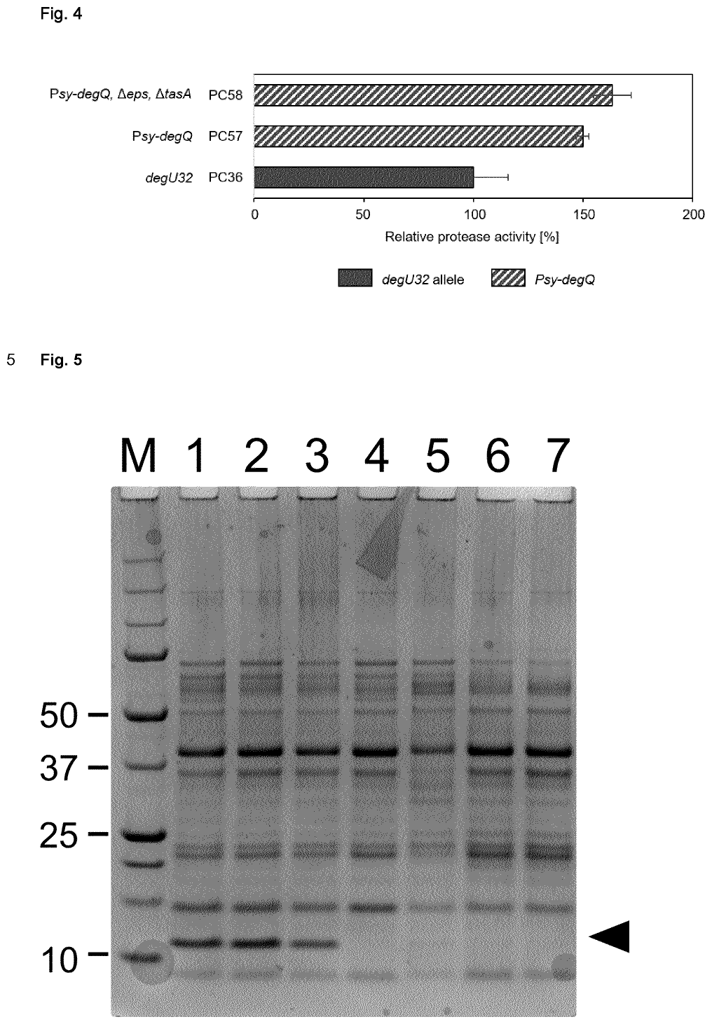

The protease yield of Bacillus licheniformis strain PC36 carrying the degU32 (H12L) mutation was set to 100% and the protease yield of the other Bacillus licheniformis strains referenced to PC36 accordingly. In FIG. 4 the relative protease activity in percent of the mutant Bacillus licheniformis strains for the 72 h timepoints of fed-batch cultivation is plotted in comparison to the Bacillus licheniformis strain PC36 (degU32 (H12L)). Introduction of an additional copy of the degQ gene under control of a strong constitutive promoter next to the endogenous natural degQ gene into Bacillus licheniformis M409 results in Bacillus licheniformis strain PC57. The Bacillus licheniformis strain PC57 shows an even 40% increase of protease yield compared to the Bacillus licheniformis strain PC36 (degU32 (H12L)). Analogous to the strain PC41 in Example 1 combining the degU32 mutation with inactivation of the tasA and eps genes, Bacillus licheniformis strain PC58 was constructed by, introduction of an additional copy of the degQ gene under control of a strong constitutive promoter into Bacillus licheniformis strain PC40, which carries an inactived tasA and eps operon. The resulting Bacillus licheniformis strain PC58 showed a further increase of protease expression by factor 1.15 as compared to the already improved Bacillus licheniformis strain PC57.

### Table Biofilm

TABLE A
Biofilm genes of Bacillus subtilis and Bacillus licheniformis (PRT: Protein)

| Gene | | Bacillus subtilis | | Bacillus licheniformis | |
|---|---|---|---|---|---|
| name | Function | DNA | PRT | DNA | PRT |
| bslA | Biofilm surface | SEQ ID 64 | SEQ ID 87 | SEQ ID 110 | SEQ ID 133 |
| | layer | | | | |
| epsA | Biofilm formation | SEQ ID 65 | SEQ ID 88 | SEQ ID 111 | SEQ ID 134 |
| epsB | Biofilm formation; | SEQ ID 66 | SEQ ID 89 | SEQ ID 112 | SEQ ID 135 |
| | protein tyrosinee | | | | |
| | kinase | | | | |
| epsC | UDP-N- | SEQ ID 67 | SEQ ID 90 | SEQ ID 113 | SEQ ID 136 |
| | acetylglucosamine | | | | |
| | 4,6-dehydratase | | | | |
| epsD | Biofilm formation | SEQ ID 68 | SEQ ID 91 | SEQ ID 114 | SEQ ID 137 |
| epsE | Glycosyltransferase | SEQ ID 69 | SEQ ID 92 | SEQ ID 115 | SEQ ID 138 |
| epsF | Biofilm formation | SEQ ID 70 | SEQ ID 93 | SEQ ID 116 | SEQ ID 139 |
| epsG | Biofilm formation | SEQ ID 71 | SEQ ID 94 | SEQ ID 117 | SEQ ID 140 |
| epsH | Undecaprenyl | SEQ ID 72 | SEQ ID 95 | SEQ ID 118 | SEQ ID 141 |
| | (UnDP) priming | | | | |
| | UDP-N-acetyl- | | | | |
| | glucosamine | | | | |
| | transferase | | | | |
| epsI | Glycosyltransferase | SEQ ID 73 | SEQ ID 96 | SEQ ID 119 | SEQ ID 142 |
| epsJ | UDP-N-acetyl- | SEQ ID 74 | SEQ ID 97 | SEQ ID 120 | SEQ ID 143 |
| | glucosamine | | | | |
| | transferase | | | | |
| epsK | poly-N- | SEQ ID 75 | SEQ ID 98 | SEQ ID 121 | SEQ ID 144 |
| | acetylglucosamine | | | | |
| | exporter | | | | |
| epsL | Biofilm formation | SEQ ID 76 | SEQ ID 99 | SEQ ID 122 | SEQ ID 145 |
| epsM | UDP-2,4,6- | SEQ ID 77 | SEQ ID 100 | SEQ ID 123 | SEQ ID 146 |
| | trideoxy-2- | | | | |
| | acetamido-4-amino | | | | |
| | glucose acetyl- | | | | |
| | transferase | | | | |
| epsN | UDP-2,6-dideoxy | SEQ ID 78 | SEQ ID 101 | SEQ ID 124 | SEQ ID 147 |
| | 2-acetamido 4-keto | | | | |
| | glucose | | | | |
| | aminotransferase | | | | |
| epsO | Biofilm formation | SEQ ID 79 | SEQ ID 102 | SEQ ID 125 | SEQ ID 148 |
| tapA | TasA anchoring/ | SEQ ID 80 | SEQ ID 103 | SEQ ID 126 | SEQ ID 149 |
| | assembly | | | | |
| | protein | | | | |
| sipW | Signal peptidase I | SEQ ID 81 | SEQ ID 104 | SEQ ID 127 | SEQ ID 150 |
| tasA | Major component | SEQ ID 82 | SEQ ID 105 | SEQ ID 128 | SEQ ID 151 |
| | of biofilm | | | | |
| phrG | Phosphatase | SEQ ID 83 | SEQ ID 106 | SEQ ID 129 | SEQ ID 152 |
| | (RapG) regulator | | | | |
| rapG | Response regulator | SEQ ID 84 | SEQ ID 107 | SEQ ID 130 | SEQ ID 153 |
| | aspartate | | | | |
| | phosphatase | | | | |
| remA | Transcriptional | SEQ ID 85 | SEQ ID 108 | SEQ ID 131 | SEQ ID 154 |
| | regulator of the | | | | |
| | extracellular matrix | | | | |
| | genes | | | | |
| remB | Involved in the activation of | SEQ ID 204 | SEQ ID 205 | SEQ ID 206 | SEQ ID 207 |
| | extracellular matrix genes | | | | |
| slrA | Anti-repressor of | SEQ ID 86 | SEQ ID 109 | SEQ ID 132 | SEQ ID 155 |
| | SlrR | | | | |
| degQ | Pleiotropic | SEQ ID 156 | SEQ ID 160 | SEQ ID 166 | SEQ ID 170 |
| | regulator | | | | |
| degR | Positive effector of | SEQ ID 157 | SEQ ID 161 | SEQ ID 167 | SEQ ID 171 |
| | DegU-phosphate | | | | |
| | stability | | | | |
| degS | Two-component | SEQ ID 158 | SEQ ID 162 | SEQ ID 168 | SEQ ID 172 |
| | sensor kinase | | | | |
| degU | Two-component | SEQ ID 159 | SEQ ID 163 | SEQ ID 169 | SEQ ID 173 |
| | response regulator - | | | | |
| | transcriptional | | | | |
| | activator | | | | |

### Example 3: Identification of Conserved Amino Acid Position within RemA

Conserved positions of amino acids in a protein sequence of interest may be determined as follows:

In a first step, create a multiple sequence alignment with the sequence of interest and sequences from a database, preferably using program HHblits (preferably version 3.3.0) acting on the UniRef30 database (preferably version 2020_06) with using default parameters.

HHblits is part of the HH-suite (Steinegger M, Meier M, Mirdita M, Vohringer H, Haunsberger S J, and Söding J (2019) HH-suite3 for fast remote homology detection and deep protein annotation, BMC Bioinformatics, 473) and can for example be downloaded from https://github.com/soedinglab/hh-suite/.

Database UniRef30 (Mirdita M, von den Driesch L, Galiez C, Martin M J, Söding J, Steinegger M. Uniclust databases of clustered and deeply annotated protein sequences and alignments. Nucleic Acids Res. 2017 Jan. 4; 45(D1): D170-D176.) can for example be downloaded from https://uniclust.mmseqs.com/.

To facilitate subsequent statistic calculations on each position in the alignment, the resulting alignment can also be converted to FASTA format. For example, the A3M alignment format can be converted to FASTA format with tool “reformat.pl”, which is also included within the HH-Suite, using the −r parameter.

In a second step, for each alignment position, the information content (IC) value then shall be computed as value R_Sequence (I) as is described by Schneider, T. D.; Stephens, R. M. Sequence Logos: A New Way to Display Consensus Sequences. Nucleic Acids Res. 1990, 18 (20), 6097-6100, with using 20 states for amino acid sequences.

A conserved position is defined as having an information content of 2.0 or higher. Table 4 lists the IC values of the multiple sequence alignment (MAS) at the amino acid positions in reference to the query sequence of RemA (SEQ ID 154).

TABLE 4

| | | IC calculated from | |
|---|---|---|---|
| AA pos. of | AA of SEQ ID 154 | MAS at given AA | |
| SEQ ID 154 | at given position | position | C |
| 1 | MET | 2.97 | * |
| 2 | THR | 0.94 | |
| 3 | ILE | 1.24 | |
| 4 | LYS | 1.72 | |
| 5 | LEU | 3.18 | * |
| 6 | ILE | 2.92 | * |
| 7 | ASN | 3.46 | * |
| 8 | ILE | 3.56 | * |
| 9 | GLY | 4.17 | * |
| 10 | PHE | 3.52 | * |
| 11 | GLY | 3.78 | * |
| 12 | ASN | 3.82 | * |
| 13 | ILE | 1.77 | |
| 14 | ILE | 3.48 | * |
| 15 | SER | 1.94 | |
| 16 | ALA | 2.35 | * |
| 17 | ASN | 1.78 | |
| 18 | ARG | 3.5 | * |
| 19 | LEU | 2.78 | * |
| 20 | ILE | 2.85 | * |
| 21 | SER | 2.91 | * |
| 22 | ILE | 3.33 | * |
| 23 | VAL | 3.08 | * |
| 24 | SER | 2.56 | * |
| 25 | PRO | 3.42 | * |
| 26 | GLU | 2.31 | * |
| 27 | SER | 3.66 | * |
| 28 | ALA | 3.43 | * |
| 29 | PRO | 3.84 | * |
| 30 | ILE | 2.5 | * |
| 31 | LYS | 3.75 | * |
| 32 | ARG | 3.98 | * |
| 33 | MET | 2.5 | * |
| 34 | ILE | 2.49 | * |
| 35 | GLN | 2.07 | * |
| 36 | ASP | 1.97 | |
| 37 | ALA | 3.4 | * |
| 38 | ARG | 3.03 | * |
| 39 | ASP | 2.03 | * |
| 40 | ARG | 1.34 | |
| 41 | GLY | 2.43 | * |
| 42 | MET | 1.15 | |
| 43 | LEU | 3.25 | * |
| 44 | ILE | 3.33 | * |
| 45 | ASP | 4.12 | * |
| 46 | ALA | 3.46 | * |
| 47 | THR | 4 | * |
| 48 | TYR | 2.01 | * |
| 49 | GLY | 4.26 | * |
| 50 | ARG | 3.89 | * |
| 51 | ARG | 3.14 | * |
| 52 | THR | 3.95 | * |
| 53 | ARG | 3.43 | * |
| 54 | ALA | 3.17 | * |
| 55 | VAL | 3.29 | * |
| 56 | VAL | 3.13 | * |
| 57 | ILE | 2.83 | * |
| 58 | MET | 2.58 | * |
| 59 | ASP | 3.53 | * |
| 60 | SER | 3.31 | * |
| 61 | ASP | 2.19 | * |
| 62 | HIS | 3.21 | * |
| 63 | ILE | 2.96 | * |
| 64 | ILE | 3.07 | * |
| 65 | LEU | 3.92 | * |
| 66 | SER | 3.79 | * |
| 67 | ALA | 3.28 | * |
| 68 | VAL | 2.45 | * |
| 69 | GLN | 2.56 | * |
| 70 | PRO | 2.96 | * |
| 71 | GLU | 3.48 | * |
| 72 | THR | 3.6 | * |
| 73 | VAL | 2.73 | * |
| 74 | ALA | 2.31 | * |
| 75 | GLN | 1.36 | |
| 76 | ARG | 3.95 | * |
| 77 | LEU | 1.99 | |
| 78 | SER | 0.54 | |
| 79 | VAL | 0.93 | |
| 80 | LYS | 0.57 | |
| 81 | GLU | 0.65 | |
| 82 | GLU | 0.59 | |
| 83 | ILE | 0.83 | |
| 84 | MET | 0.83 | |
| 85 | ASP | 1.71 | |
| 86 | GLU | 1.63 | |
| 87 | GLY | 1.45 | |
| 88 | GLN | 0 | |
| 89 | GLY | 0 | |

Pos. = position
AA = amino acid
IC = information content
C. = conserved amino acid with IC > 2.0; marked with * (asterix)
MAS. = multiple sequence alignment

### Example 4: Generation of B. licheniformis Enzyme Expression Strains

Bacillus licheniformis strains as listed in Table 5 were made competent as described above. Protease expression plasmid pUK56 (WO2019016051) was isolated from B. subtilis Bs #056 strain (WO2019016051) to carry the B. licheniformis specific DNA methylation pattern. Plasmids were transformed in the indicated strains and plated on LB-agar plates with 20 μg/μl kanamycin. Individual clones were analyzed for correctness of the plasmid DNA by restriction digest and functional enzyme expression was assessed by transfer of individual clones on LB-plates with 1% skim milk for clearing zone formation of protease producing strains. The resulting B. licheniformis expression strains are listed in Table 5.

TABLE 5
Overview on B. licheniformis expression strains

| B. licheniformis | Expression | B. licheniformis |
|---|---|---|
| Expression strain | plasmid | strain |
| BES#130 | pUK56 | Bli#008 |
| BES#131 | pUK56 | Bli#030 |

### Example 5: Cultivation of Bacillus licheniformis Protease Expression Strains

Bacillus licheniformis strains from Example 4 were cultivated in a fermentation process using a chemically defined fermentation medium.

The following macroelements were provided in the fermentation process:

| | | Concentration [g/L |
|---|---|---|
| Compound | Formula | initial volume] |
| Citric acid | C₆H₈O₇ | 3.0 |
| Calcium sulfate | CaSO₄ | 0.7 |
| Monopotassium phosphate | KH₂PO₄ | 25 |
| Magnesium sulfate | MgSO₄*7H₂O | 4.8 |
| Sodium hydroxide | NaOH | 4.0 |
| Ammonia | NH₃ | 1.3 |

The following trace elements were provided in the fermentation process:

| Trace element | Symbol | Concentration [mM] |
|---|---|---|
| Manganese | Mn | 24 |
| Zinc | Zn | 17 |
| Copper | Cu | 32 |
| Cobalt | Co | 1 |
| Nickel | Ni | 2 |
| Molybdenum | Mo | 0.2 |
| Iron | Fe | 38 |

The fermentation was started with a medium containing 8 g/l glucose. A solution containing 50% glucose was used as feed solution. The pH was adjusted during fermentation using ammonia. In both experiments, the total amount of added chemically defined carbon source was kept above 200 g per liter of initial medium. Fermentations were carried out under aerobic conditions for a duration of more than 70 hours.

At the end of the fermentation process, samples were withdrawn and the protease activity determined photometrically: proteolytic activity was determined by using Succinyl-Ala-Ala-ProPhe-p-nitroanilide (Suc-AAPF-pNA, short AAPF; see e.g. DelMar et al. (1979), Analytical Biochem 99, 316-320) as substrate. pNA is cleaved from the substrate molecule by proteolytic cleavage at 30° C., pH 8.6 TRIS buffer, resulting in release of yellow color of free pNA which was quantified by measuring at OD405.

The protease yield was calculated by dividing the product titer by the amount of glucose added per final reactor volume. The protease yield of strain BES #130 was set to 100% and the protease yield of the strain BFS #131 referenced to BES #130 accordingly (Table 6). B. licheniformis expression strain BES #131, with the mutated remA gene (resulting in an altered RemA protein comprising the mutations R18W und P29S) showed 10% improvement in the protease yield compared to B. licheniformis expression strain BES #130.

TABLE 6
Protease yield of Bacillus licheniformis expression strains

| B. licheniformis | Protease |
|---|---|
| Expression strain | yield [%] |
| BES#130 | 100 |
| BES#131 | 110 |

### Example 6

Bacillus licheniformis strains with genetic modifications (as indicated in Table 1) were cultivated in a microtiter plate-based fed-batch process (Habicher et al., 2019 Biotechnol J.; 15(2)).

TABLE 7
Bacillus licheniformis mutant strains

| Bacillus licheniformis | |
|---|---|
| strain name | Genotype |
| DSM641 (P300) | Wild type |
| M309 | P300 DRe, DaprE, DsigF, Dpga |
| P311 | M309 DforD |
| P312 | M309 CAT::Psy-DegQ |

‘D’ denotes deleted;
RE: restriction endonuclease

All cultivations were conducted in an orbital shaker with a diameter of 25 mm (Innova 42, New Brunswick Scientific, Eppendorf AG; Hamburg, Germany) at 30° C. and 400 rpm. Strains were cultivated in two subsequent precultures in FlowerPlates (MTP-48-OFF, m2p-labs GmbH) for synchronization of growth. The first preculture was carried out in 800 μl TB medium inoculated with a fresh single colony from the strain streaked onto LB agar plates. After 20 h, the second preculture containing 800 μl V3 minimal medium (Meissner et al., 2015, Journal of industrial microbiology & biotechnology 42 (9): 1203-1215) as inoculated with 8 μl of the first preculture and cultivated for 24 h. Microtiter plate-based fed-batch main cultivations were conducted using 48-well round- and deep-well-microtiter plates with glucose-containing polymer on the bottom of each well (FeedPlate, article number: SMFP08004, Kuhner Shaker GmbH; Herzogenrath, Germany). 70 μl of the second preculture were used to inoculate 700 μl V3 minimal medium without glucose. Main cultures were incubated for 72 h. Precultures were covered with a sterile gas-permeable sealing foil (AeraSeal film, Sigma-Aldrich) to avoid contamination. FeedPlates were sealed with a sterile gas-permeable, evaporation reducing foil (F-GPR48-10, m2p-labs GmbH) to reduce evaporation and to avoid contamination.

At the end of the cultivation process, supernatants of cultivations were prepared by centrifugation and sterile filtration with a 0.2 μm filter. For SDS-PAGE analysis equal amounts of supernatants were directly resuspended with 2×SDS sample buffer and boiled for 10 min at 95° C., following loading on SDS-PAGE gel.

For SDS PAGE analysis a 4-12% BIS-TRIS-Gel (NuPAGE), 10 Well was used with MES Running Buffer. Staining was performed using Comassie Brillant Blue G250 dissolved in acetic acid/ethanol with destaining with performed with acetic acid/ethanol.

FIG. 5 shows the SDS PAGE of supernatants of cultivations of Bacillus licheniformis M309 strain in comparison to Bacillus licheniformis P311 strain with deleted forD gene, and Bacillus licheniformis P312 strain comprising the forD gene with an additional copy of the degQ gene under control of a constitutive promoter with increased DegQ expression levels. Surprisingly, in Bacillus licheniformis P312 strain the amount of Formosin D is strongly reduced compared to the control strain M309. As control, Bacillus licheniformis P311 strain with deleted forD gene does not show the Formosin D band on the SDS-PAGE. FIG. 5: SDS-PAGE of supernatant from B. licheniformis strains after 72 h of simulated fed-batch cultivation. 1-3=control strain; 4=ForD deletion strain; 5-7=DegQ overexpression strain; M=Precision Plus Protein Standard with indicated size in kDa of selected bands; filled arrowhead highlights the ForD protein band.

### Example 7

Bacillus licheniformis strains with genetic modifications (as indicated in Table 8) were cultivated in a microtiter plate-based fed-batch process as described in Example 6.

TABLE 8
Bacillus licheniformis mutant strains

| Bacillus licheniformis | |
|---|---|
| strain name | Genotype |
| DSM641 (P300) | Wild type |
| M309 | P300 DRe, DaprE, DsigF, Dpga |
| M409 | M309 pga::BLAP |
| P313 | M409 DforD |
| PC57 | M409 CAT::Psy-DegQ |

‘D’ denotes deleted;
RE: restriction endonuclease

At the end of the cultivation process, supernatants of cultivations were prepared by centrifugation and sterile filtration with a 0.2 μm filter. For SDS-PAGE analysis supernatants were TCA precipitated followed by SDS-PAGE with Coomassie Blue Staining. For TCA precipitation the sample was adjusted to 13.3% TCA (w/w), kept on ice for 10 min, then sedimented using a table top centrifuge, the pellet washed using aceton. The pellet was then resuspended using 1×SDS sample buffer. Prior SDS-PAGE the samples were boiled for 10 min at 95° C. For SDS-PAGE analysis equal amounts of supernatants were loaded onto the SDS-PAGE gel. For SDS PAGE analysis a 4-12% BIS-TRIS-Gel (NuPAGE), 10 Well was used with MES Running Buffer. Staining was performed using Comassie Brillant Blue G250 dissolved in acetic acid/ethanol with destaining with performed with acetic acid/ethanol.

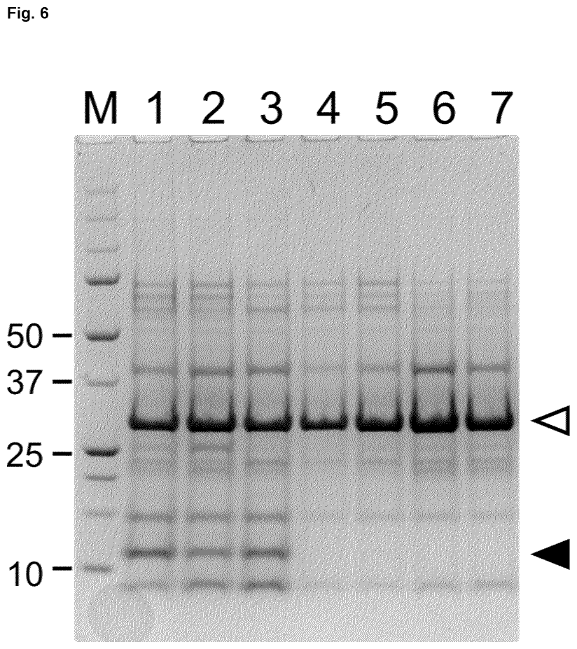

FIG. 6 shows the SDS PAGE of supernatants of cultivations of Bacillus licheniformis M409 strain in comparison to Bacillus licheniformis P313 strain with deleted forD gene, and Bacillus licheniformis PC57 strain comprising the forD gene with an additional copy of the degQ gene under control of a constitutive promoter with increased DegQ expression levels. Surprisingly, in Bacillus licheniformis PC57 strain the amount of Formosin D is strongly reduced compared to the control strain M409. As control, Bacillus licheniformis P313 strain with deleted forD gene does not show the Formosin D band on the SDS-PAGE. FIG. 6: SDS-PAGE of supernatant from B. licheniformis protease expression strains after 72 h of simulated fed-batch cultivation. 1-3=control strain; 4=ForD deletion strain; 5-7=DegQ overexpression strain; M=Precision Plus Protein Standard with indicated size in kDa of selected bands; filled arrowhead highlights the ForD protein band; open arrowhead indicates the heterologous protease.

1. A modified Bacillus host cell comprising
i) a mutation which reduces the amount of exopolymeric substances (EPS) and/or a mutation which reduces the amount of the biofilm extracellular matrix component TasA, and
ii) a mutation which increases the amount of phosphorylated DegU as compared to a control cell.

2. The modified Bacillus host cell of claim 1, wherein the mutation which reduces the amount of exopolymeric substances (EPS) is a mutation that causes a reduced expression of the epsA-O operon, and wherein the mutation which reduces the amount of the biofilm extracellular matrix component TasA is a mutation that causes a reduced expression of the tapA-sipW-tasA operon.

3. The modified Bacillus host cell of claim 2, wherein the mutation is selected from
a) a mutation that inactivates the tapA-sipW-tasA operon,
b) a mutation that inactivates the epsA-O operon,
c) a mutation that inactivates the gene remA,
d) a mutation that inactivates the gene remB, and
e) a mutation that inactivates the slrA gene.

4. The modified Bacillus host cell of claim 3, wherein the mutation that inactivates the tapA-sipW-tasA operon is a deletion of tapA-sipW-tasA operon, or of a portion thereof.

5. The modified Bacillus host cell of claim 3, wherein the mutation that inactivates the epsA-O operon is a deletion of epsA-O operon, or of a portion thereof.

6. The modified Bacillus host cell of claim 3, wherein the mutation that inactivates the gene remA or remB is a missense mutation of said gene or a full or partial deletion thereof.

7. The modified Bacillus host cell of claim 3, wherein the mutation that inactivates the sirA gene, is a deletion of the slrA gene.

8. The modified Bacillus host of claim 5, wherein the mutation which increases the amount of phosphorylated DegU is selected from
u1) a mutation causing increased expression of at least one of the genes selected from the group consisting of degU, degS, degQ and degR,
u2) a mutation causing decreased expression of the rapG gene or an increased expression of the phrG gene,
u3) a mutation stabilizing the DegU phosphorylation state, such as a degU32 mutation,
u4) a mutation increasing autophosphorylation activity of the DegS protein, such as a DegS-S76D mutation, and
u5) a mutation reducing phosphatase activity of the DegS protein, such as a degS200 mutation.

9. The modified Bacillus host cell of claim 8, wherein the mutation is a degU32 mutation.

10. The modified Bacillus host cell of claim 8, wherein the mutation is a mutation that causes increased expression of degQ, such as the introduction and expression of an additional copy of the degQ gene in the host cell.

11. The modified Bacillus host cell of claim 1, wherein the host cell belongs to the species Bacillus pumilus, Bacillus velezensis, Bacillus amyloliquefaciens, Bacillus alcalophilus, Bacillus licheniformis, Bacillus paralicheniformis, Bacillus lentus, Bacillus clausii, Bacillus halodurans, Bacillus megaterium, Bacillus methanolicus, Geobacillus stearothermophilus (Bacillus stearothermophilus), Bacillus mojavensis, Bacillus globigii, or Bacillus subtilis.

12. The modified Bacillus host cell of claim 1, wherein the host cell comprises an expression cassette for a polypeptide of interest.

13. The modified Bacillus host cell of claim 12, wherein the polypeptide of interest is an enzyme, such as an enzyme selected from the group consisting of amylase, protease, lipase, phospholipase, mannanase, phytase, xylanase, phosphatase, glucoamylase, nuclease, galactosidase, endoglucanase and cellulase.

14. A method for producing a polypeptide of interest, comprising
a) providing the modified Bacillus host cell of claim 12,
b) cultivating the host cell under conditions which allow for the expression of the polypeptide of interest, and
c) optionally isolating the polypeptide of interest from the cultivation medium.

15. The method of claim 14, wherein the cultivation in step b) is carried out as fed batch cultivation.


SEQUENCE LISTING (218 sequences)

SEQ ID NO: 1 - DNA, 507 bp, Artificial Sequence
  5-prime region of pga genes with flanking BsaI sites
ggtctcagagaataattcctatcagatttttgtcagcgttttaagtatgcgctttcaaaattcgacattatgatttttgtacttttctccctatttcagatgaaaggttgaaggatggattcaggatctcttttttcccccggccgcaatcccctttaaatgggctgtcagccgggatgtctcttttcttttagtattttcgctcttttcgaacaggttaaaaagtcaccaatgttccctcttcaaaacaaatccgtcctatgacaatcagaccatctatcaaaaaatgaaaatctcttttagcataaagcgggattcattgacaaatccaaatattttgtgagtcgttcgatccaccttatttcttcagattttaggttaaaatcctaggatttaaaaagccattaatttttttcaatgataattcatcaaactttaacatttttaatttttcttactattctcattgttactgtattcgccattaaacgcccctctcatgagacc

SEQ ID NO: 2 - DNA, 560 bp, Artificial Sequence
  3-prime region of pga genes with flanking BsaI sites
ggtctcaacccagttaaaaataaagacatccaaagcaccgcaattccacaaaacaaaaccaactttttgtttgccgctttttttatcaattgatctgttcctcctcttctttcctctttcatcgtcattcaactgggatatttaaaggactttttttgcatgtacagcagcgtcatagcgagaagaaacgctcagcacagcatgatcagctttttcacctaagcgcggattaaaaagatgaagggaaatattctaaagaggtcatgaacatgaggtgcggttcccatcccgaattggtaaggtgatgcattttcaaattttgcttcagtcgaaatttgtcagtttttattcccttcaaaagaatacaataaacatgaaccccaaaaccatgtatatatcctcctgattttctataaaactttatacactgaccggttaagaaaattgtcgtgtttccctttcaaaccggccgcttttcacaggtcttttataaccgggatcagcacacaaaaaagagagcgccggcggcactctcttttgcactgttatgcgacgagacc

SEQ ID NO: 3 - DNA, 52 bp, Artificial Sequence
  Oligonucleotide HomA bslA
ggtggtggtctctacccggccaacgaggcctcaatcataaactcggttcgtg

SEQ ID NO: 4 - DNA, 40 bp, Artificial Sequence
  Oligonucleotide HomA bslA
ggagcttttttgcctatttacacagcaattcccccaagtc

SEQ ID NO: 5 - DNA, 44 bp, Artificial Sequence
  Oligonucleotide HomB bslA
gacttgggggaattgctgtgtaaataggcaaaaaagctccaaag

SEQ ID NO: 6 - DNA, 50 bp, Artificial Sequence
  Oligonucleotide HomB bslA
ggtggtggtctcttgagggccttattggccatgtctgcttctatccgatg

SEQ ID NO: 7 - DNA, 24 bp, Artificial Sequence
  Oligonucleotide Spacer bslA
tacgaactgaatctgtctaccgcc

SEQ ID NO: 8 - DNA, 24 bp, Artificial Sequence
  Oligonucleotide Spacer bslA
aaacggcggtagacagattcagtt

SEQ ID NO: 9 - DNA, 32 bp, Artificial Sequence
  Oligonucleotide PCR-amplification of pE194
tatatacagctggattcacaaaaaataggcac

SEQ ID NO: 10 - DNA, 33 bp, Artificial Sequence
  Oligonucleotide PCR-amplification of pE194
tatatacagctggattatgtcttttgcgcagtc

SEQ ID NO: 11 - DNA, 30 bp, Artificial Sequence
  Oligonucleotide PCR-amplification of functional mRFP cassette
tatatggatccgtaatcagggtatcgaggc

SEQ ID NO: 12 - DNA, 31 bp, Artificial Sequence
  Oligonucleotide PCR-amplification of functional mRFP cassette
tatatggatccctcattaggcgggctactaa

SEQ ID NO: 13 - DNA, 2633 bp, Bacillus licheniformis
  misc_feature (1)..(2633): P300 DSM641 RMS region
tttttgcctacttatttatttgttttcattttcaaattcatcataagaacccatttcacaaaaatcaatggagtaatgttgttcgctgtgtttataaacaattactgagtcaatatttagtctttccagcatttcataggttagaagttctttttcctctaagttcataacttgtcttagtagccattctccaagagcactatttggattagacataagtgctttactattgtcttggcataccttggctgataaaagcgatttgtctggcaagcgtaactgaaaaggtttatcacgagctgggaaaaatgttgggaatacattatgaatccattttggaattggtatataaatctcgttagggtttcgtggtcgacctaaagcattccattggtttagaccgcttttttctggtacatgacgctttgagccacggtctgaaaagagtggaagaataacgtgctcaaggttttcaaaaggattgacagttggtgctggaatttttggaatttcaaagccaaatagtttagccaattcatgataaggattttctaagatttcaacattaatttcttcaataggtttatcagtgataaaacgcttataaagggtgctcttagtgacattaaagctgtattcgtgtagaccgtcttcaaaggtgattgtatttctgttgttacttactttcacatttgtaattgaggagatttcaaccaagtccattggctcttcaaaaataagaattttccctggctttcttgttacacagtggtatatcattgaatcaataccatatgttcttttagtaaattcaattctctcgttacggagagaagcaaccgtgtttattagctcttttggagatttcccacgatacaagtctgagtctttattgaattcagctactttttgaagagtatgaccattaccatgaagaaaagtcttaataccaattccgacacgatttaatgaagcgtcagcagaacagtctgacctccccaagttttcagctccaaatgcttcacaaaaagcattttccacattccttgagaccaaataaggcgagtcactttcagagaacaaattggatagcgaaccagttgagcggagcatttgtttgtatgtagtgcagttgatggctggttgattagtatagaacattatttttcctcctcttttatgcttgtcatttcttctttcagacccaaaaggtagtcagctgatacgttcaatgtttcagctattcttttgaaagtgtccaatgatggagttctattttcactttcatatagtgaccaagtgcttctagtgaccccgactttttcagcgatttggctgggtaataacctacgagcttctcttgcattttgaatacgatttccaaggaaaggtatcatttttgcacctccaagatttgttgttttcagagtatcaccagaacccccgaaaatagtccaaagttagctaacagcaaacaaataaaaataaataagttgtttactcttagcaaacttgttactaaaatttgataaagttattcatttaatccagctcttatgctaaaattgcattagcggacaagcttaatgtttgcaaggaggtataattttgacttatcgagtaggtagtatgtttgctgggataggtggaacttgtttagggtttatccaagctggcgctaggattgtctgggcaaatgaaatagacaaaaatgcttgtattacttatagaaattattttggggatgcttacttacaagagggtgacattaacctaatagataaaaactccatacctgaactggacattttgattggaggttttccttgccaagccttctctatagctggctatcgtaaagggtttgaagatgaaaggggaaacgtgttctttcaaatattagaggtattggaagcacaaagaaatgtttatggacacttaccccaagcaataatgcttgagaatgtaaagaacttatttacacatgatagaggtaatacgtacagagtaataaaagaggctttggaagcctttggttataccgtaaaagctgaggttcttaattcaatggaatacggtaacgtgccacaaaacagagagcggatttatattgtaggttttcaagatgagagccaagctgaaaggtttagctttccagacccaattcctttaacaaatcaacttaatgatgtaattgaccgaactcggagagttgataaaagatattattatgatgaaacctctcaatattatgatatgttgcgagaagccatggacagtacagatacaacttatcaaataagacgtatatatgttcgagaaaatagaagcaatgtttgtcctacactgacagcgaatatgggaactggagggcataatgttcctattgtattagactttgaaaataatataagaaaactaacaccagaagaatgcttactattgcaaggtttcccagctgactatcattttccagaaggcatggcaaacactcacaaatataaacaagctggtaactctgttacggtgccagttataagaagaattgccactaatattattagcgtattgaacattggaatgaatataaatcaagaacatgaatatgcaatagctgaataagaccaactaat

SEQ ID NO: 14 - DNA, 1146 bp, Bacillus licheniformis
  misc_feature (1)..(1146): DNA encoding Restrictase P300 DSM641
atgttctatactaatcaaccagccatcaactgcactacatacaaacaaatgctccgctcaactggttcgctatccaatttgttctctgaaagtgactcgccttatttggtctcaaggaatgtggaaaatgctttttgtgaagcatttggagctgaaaacttggggaggtcagactgttctgctgacgcttcattaaatcgtgtcggaattggtattaagacttttcttcatggtaatggtcatactcttcaaaaagtagctgaattcaataaagactcagacttgtatcgtgggaaatctccaaaagagctaataaacacggttgcttctctccgtaacgagagaattgaatttactaaaagaacatatggtattgattcaatgatataccactgtgtaacaagaaagccagggaaaattcttatttttgaagagccaatggacttggttgaaatctcctcaattacaaatgtgaaagtaagtaacaacagaaatacaatcacctttgaagacggtctacacgaatacagctttaatgtcactaagagcaccctttataagcgttttatcactgataaacctattgaagaaattaatgttgaaatcttagaaaatccttatcatgaattggctaaactatttggctttgaaattccaaaaattccagcaccaactgtcaatccttttgaaaaccttgagcacgttattcttccactcttttcagaccgtggctcaaagcgtcatgtaccagaaaaaagcggtctaaaccaatggaatgctttaggtcgaccacgaaaccctaacgagatttatataccaattccaaaatggattcataatgtattcccaacatttttcccagctcgtgataaaccttttcagttacgcttgccagacaaatcgcttttatcagccaaggtatgccaagacaatagtaaagcacttatgtctaatccaaatagtgctcttggagaatggctactaagacaagttatgaacttagaggaaaaagaacttctaacctatgaaatgctggaaagactaaatattgactcagtaattgtttataaacacagcgaacaacattactccattgatttttgtgaaatgggttcttatgatgaatttgaaaatgaaaacaaataa

SEQ ID NO: 15 - DNA, 1022 bp, Artificial Sequence
  Fusion of 5-prime and 3-prime regions of restrictase gene with flanking BsaI sites
ggtctcgacccaacttctataaatgtaacgatacgatttattgtttcattaaagtcttcctttatttcatgttcccatattcttttaatgttccaatccttttcctcgtaatatttattaacttccttatctctttttttatttctttcgagttttttctcccaatattccgtattactttttggtatattcccgtgtttttcacacgcatgccagaaacaagaatcaatgaatatgactattttatatttctgtattactatatctggactaccgtataatttcttaacattttttcggaatcttattccacggtgccatagttctttagtaaccttatcttctaattttgaacgagatttgattgcctgcatgttttttcttctttgttcttttgaaaccgtgtcagtcatagaagagtcctccaaagccacaataattgtattctataaacgaggaagcaagccctcaagcttaccccctcttagttccttttttgcctacttatttatatttttcctcctcttttatgcttgtcatttcttctttcagacccaaaaggtagtcagctgatacgttcaatgtttcagctattcttttgaaagtgtccaatgatggagttctattttcactttcatatagtgaccaagtgcttctagtgaccccgactttttcagcgatttggctgggtaataacctacgagcttctcttgcattttgaatacgatttccaaggaaaggtatcatttttgcacctccaagatttgttgttttcagagtatcaccagaacccccgaaaatagtccaaagttagctaacagcaaacaaataaaaataaataagttgtttactcttagcaaacttgttactaaaatttgataaagttattcatttaatccagctcttatgctaaaattgcattagcggacaagcttaatgtttgcaaggaggtataattttgacttatcgagtaggtagtatgtttgctgggataggtggaacttgtttaggctcaggagacc

SEQ ID NO: 16 - DNA, 21 bp, Artificial Sequence
  Oligonucleotide PCR-amplification of restrictase genomic region
gacaatccccttttactgacc

SEQ ID NO: 17 - DNA, 24 bp, Artificial Sequence
  Oligonucleotide PCR-amplification of restrictase genomic region
ctatttcatttgcccagacaatcc

SEQ ID NO: 18 - DNA, 1363 bp, Artificial Sequence
  Fusion of 5-prime and 3-prime regions of pga genes with flanking BsaI sites
ggtctcgacccgaacactgaaattttagaccgggctgggatatcgagacatatataggggcgtttaatggcgaatacagtaacaatgagaatagtaagaaaaattaaaaatgttaaagtttgatgaattatcattgaaaaaaattaatggctttttaaatcctaggattttaacctaaaatctgaagaaataaggtggatcgaacgactcacaaaatatttggatttgtcaatgaatcccgctttatgctaaaagagattttcattttttgatagatggtctgattgtcataggacggatttgttttgaagagggaacattggtgactttttaacctgttcgaaaagagcgaaaatactaaaagaaaagagacatcccggctgacagcccatttaaaggggattgcggccgggggaaaaaagagatcctgaatccatccttcaacctttcatctgaaatagggagaaaagtacaaaaatcataatgtcgaattttgaaagcgcatacttaaaacgctgacaaaaatctgataggaattaagaactttcgatttccaaaaatatcaataaaaagataggcattaatgactcgggcgaggtgatctttgtcacggaaaatttcgtcgtcttctgttacataatgccgattgtgatttcatagtgaaccctgatcccggttataaaagacctgtgaaaagcggccggtttgaaagggaaacacgacaattttcttaaccggtcagtgtataaagttttatagaaaatcaggaggatatatacatggttttggggttcatgtttattgtattcttttgaagggaataaaaactgacaaatttcgactgaagcaaaatttgaaaatgcatcaccttaccaattcgggatgggaaccgcacctcatgttcatgacctctttagaatatttcccttcatctttttaatccgcgcttaggtgaaaaagctgatcatgctgtgctgagcgtttcttctcgctatgacgctgctgtacatgcaaaaaaagtcctttaaatatcccagttgaatgacgatgaaagaggaaagaagaggaggaacagatcaattgataaaaaaagcggcaaacaaaaagttggttttgttttgtggaattgcggtgctttggatgtctttatttttaacgaatcataatgatgtacgcgccgatacgatcggcgagaaaatagcggaaactgccagacagcttgagggtgcgaaatacagctacggcggagagaagccgaaaacggggtttgactcgtcaggctttgtgcaatatgtgtttcaatcgctcgatattacgcttccgagaacggtaaaggaacaatcgactcttggctcaggagacc

SEQ ID NO: 19 - DNA, 20 bp, Artificial Sequence
  Oligonucleotide PCR-amplification of pga genomic region
aaagccttctcctctctatt

SEQ ID NO: 20 - DNA, 20 bp, Artificial Sequence
  Oligonucleotide PCR-amplification of pga genomic region
ttcttgaaaaagacaaggtc

SEQ ID NO: 21 - DNA, 1027 bp, Artificial Sequence
  Fusion of 5-prime and 3-prime regions of the aprE gene with flanking BsaI sites
ggtctcgacccgaagttcttttttaacatataggtaaaacaatacgaaaaaaggcgccaagtattgaagaattgcagcagccgcggcatttcccttttcgattgaagcaaaaaacgtatattgaacagtaagcattccaaaaatggaaaatactaaaatcgaacaaatatctgtttttttcttccatatctgacacacatgttgaaaaccgtttttcattgaaacatataacaagagaatgactcccgatgccagaagcctgacagagacaagcgagccggcttcaaccgctcccctttcaaatatgtactgtgcagcgcttcccgataatccccacaatgaagcccctgcaagcaccatcaatacgcctttcacatgagctgatttcatatctttcacccgtttctgtatgcgatatattgcatattttaatagatgatcgacaaggccgcaacctccttcggcaaaaaatgatctcataaaataaatgaatagtattttcataaaatgagctcaataacatattctaacaaatagcatatagaaaaagctagtgtttttagcactagctttttcttcattctgatgaaggttgttcaatattttgaatccgttccatgatcgtcggatggccgtatttaaaaatcttgacgagaaacggcgggtttgcctcgctcagcccggcttttgagagctcttgaaacgtcgaaaccgctgcatcgctgttttgcgtcagttcaatcgcatactggtcagcagctttttcctgatgcctcgaaactgcgttcgtaaatggagacgacgcgaaagagatgacccccatcagcatcagaagaagcggaagtgcggctagatcggattttcctgcaatatgaaggcttcttccatagcggccgatgatccgcttgtacagcttgtcgatcacataaaagacagcaagggataaaagcagatacccgccaagtcctatgtaaacatgcttcatcacatagtgccccatttcgtgcgccatgatgctcaggagacc

SEQ ID NO: 22 - DNA, 21 bp, Artificial Sequence
  Oligonucleotide PCR-amplification of aprE genomic region
ccggttgtcattgatccttta

SEQ ID NO: 23 - DNA, 21 bp, Artificial Sequence
  Oligonucleotide PCR-amplification of aprE genomic region
atcctcctgcaaaaaccgtat

SEQ ID NO: 24 - DNA, 1022 bp, Artificial Sequence
  Fusion of 5-prime and 3-prime regions of sigF gene with flanking BsaI sites
ggtctcgacccaaaatcattcgccttgagcaatcagagcagcgtgcacttgaaacgttgggggtggcgtcatgaaaaatgaaatgaacattcagtttacagcgctcagccaaaatgaatcgtttgcacgggtgacagtcgctgcttttatcgctcagcttgacccgacgatggatgaactgaccgaaattaaaacggtcgtatccgaagcggtcacaaacgcgatcattcacggttatgaaaactcagggcagggaaacgtatatatttccgtcactctcgaggaccatattgtctatttaacgatccgcgacgaaggagtcggcatccctgatcttgaagaagcgcgccagcccctgttcacgacaaagcctgaactcgagcggtcgggaatgggctttacgatcatggaaaatttcatggatgatatttcgatcgactcctcacctgagatgggaaccacaatacacttaacaaagcacttatcaaaaagcaaagcgctttgcaattaatgaggctgctcatgttgcaggcagcctcggatgtccgatgaaaaaccggacgctcttgggagcgttccggttttttttgtgtggtaatttatggtcttttgcgcctttctgaatgataaatgggatgtacttcatactacaactataaccatcatataggaagtgacccagatggaacgtcaagtttttatcagactccgccaccggcttgaggcagatccggatgaattgattttgctcggccatatcgcacaggtagcgggtgaccgcggatacaaagaaaagcttgaacggctgcctatttatcaggtcagcaaagcggatcaaagcatggtggtgctggacgtgatgaaggtgattgaagctgtacataaatcgtttcctgaccttgatgtccaaaccgtcggcggttctgaaaccatcgtggagatccaatatccgaaaaggggtctgtcgcccgtgcttttcatcgccgtctggctgctcttgttcgtcggtgcctcaggagacc

SEQ ID NO: 25 - DNA, 19 bp, Artificial Sequence
  Oligonucleotide PCR-amplification of sigF genomic region
agcagctcggcggagaaat

SEQ ID NO: 26 - DNA, 21 bp, Artificial Sequence
  Oligonucleotide PCR-amplification of sigF genomic region
accttgcccgtcaccatttcg

SEQ ID NO: 27 - DNA, 865 bp, Artificial Sequence
  Functional fragment T2A lacZ cassette
gcgatgttaaggaccgtctcatctcacctgcaaggtctcatctcttttactccatctggatttgttcagaacgctcggttgccgccgggcgttttttatctaaaactagtgtcgagggtcttcggtaccgcgatttacatatgctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgccgcgtttataatgaagaccctagcaggcatcaaataaaacgaaaggctcagtcgaaagactgggcctttcgttttatctgttgtttgtcggtgaacgctctcctgagtaggacaaatccgccgccctagacagctgtcgctgagacgatcgctgagacctcgcaagttctcgccatcgcaggtgaaatctagatgtattcgc

SEQ ID NO: 28 - DNA, 35 bp, Artificial Sequence
  Oligonucleotide PCR-amplification of BLAP expression cassette
ggagaagaccttcgacctcgggacctctttccctc

SEQ ID NO: 29 - DNA, 36 bp, Artificial Sequence
  Oligonucleotide PCR-amplification of BLAP expression cassette
tccgaagacccgctattagcgtgttgccgcttctgc

SEQ ID NO: 30 - DNA, 1345 bp, Artificial Sequence
  Functional fragment T2A mRFP cassette
ggaatagatctggccaacgaggcctcgaggatccgatatcatgcatggcgcgccaccctgagacgaccctgatgcaggtgaccctgagaccttcgcgcccagctgtctagggcggcggatttgtcctactcaggagagcgttcaccgacaaacaacagataaaacgaaaggcccagtctttcgactgagcctttcgttttatttgatgcctttaattaagtaccttgtcggataaagctgtgttatattatgtcttggtgttaaatacacacgcttaacgatttatgcagagggtgctgcaggcggcagttctgtacaaaaatgacctaagcggaggaaaaaaaccattatattaggaggaaataacatggcctcttcagaggatgttattaaagaatttatgcggtttaaggtgaggatggaaggctcggtgaacggacatgagttcgaaattgagggagaaggtgaaggccgcccttatgaaggtactcagacagcgaaattgaaagtcacgaaaggcggaccgctgccgtttgcttgggacattctctcacctcaatttcaatatggctcaaaagcctacgtaaaacacccggctgacatccctgattacttaaagctatccttcccggagggctttaaatgggaacgagttatgaattttgaggacggcggcgtcgttactgtcacacaggattcttcccttcaggatggcgaatttatttacaaagtaaaacttcgtggaactaacttcccaagtgatggtcccgtgatgcaaaaaaaaacaatgggatgggaagcatctacggaacgtatgtatccggaggatggagccttaaagggtgaaatcaaaatgcgcctgaaacttaaagatggcggacactatgacgcggaagttaaaacaacatatatggctaaaaaaccagtccaactgccgggagcatataagacggatataaagttggacattaccagccataatgaagattacacgattgtggaacagtatgagagagcagagggcagacatagcacaggcgcgtaagaattaatgaaaaataagcggcagcctgcttttccatgcgggctgccgcttatcgggttattgtcgtgactgggaaaaccctggcgactagtcttggactcctgttgatagatccagtaatgacctcagaactccatctggatttgttcagaacgctcggttgccgccgggcgttttttattggtgagaatccaggggtccccaataattacgatttggtctcactcacacctgctcgtctcactcaatttaaatggcggccgcggatcctcgacgggccaataaggccagatctggatt

SEQ ID NO: 31 - DNA, 645 bp, Artificial Sequence
  Artificial promoter
gagtcgcttttgtaaatttggaaagttacacgttactaaagggaatgtagataaattattaggtatactactgacagcttccaaggagctaaagaggtccctagactctagacccggggatctctgcagtcgggaagatctggtaatgactctctagcttgaggcatcaaataaaacgaaaggctcagtcgaaagactgggcctttcgttttatctgttgtttgtcggtgaacgctctcctgagtaggacaaatccgccgctctagctaagcagaaggccatcctgacggatggcctttttgcgtttctacaaactcttgttaactctagagctgcctgccgcgtttcggtgatgaagatcttcccgatgattaattaattcagaacgctcggttgccgccgggcgttttttatgcagcaatggcaagaacgttgctctagagttaagggattttggtcatggccattcttctgtacaccaatgtcatgacatttttatctcatttggattattaaaattttgtcaaaataattttattgacaacgtcttattaacgttgataccggttaaattttatttgaacaaaaatgggctcgtgttggagaataaatgtggagaaagattaactaataaggaggacaaac

SEQ ID NO: 32 - DNA, 814 bp, Artificial Sequence
  Homology region of the mutated degU gene with flanking BsaI sites
ggtctcgacccgatttgggattgataccgacgctcagaaaatacttgaacacgatcgaagattatcatggaaaagcaaagattcatttccaatgcatcggagaatccgaagaaagaagaatagcaccgcggtttgaggttgcactattccggcttgcacaggaagcggtgacaaacgccttaaaacactccgaatcaactgaaattcatgttaaagtagaagtgacaaaagattttgtgacgctgattatcaaagacaatggaaacggctttgacttaaaagaagtaaaaggcaagaagaacaaatctttcggtctgctaggtatgaaagaaagagtcgatttgctcgaaggctcaatgacaatcgattcgaaaataggtcttgggacatttatattgattaaagttccactgtctttgtaaagataattgtaaaatagagacaaaagacatattgaccataaaagcggtgtgtttaacaatgagaatggggaggcgtagcttgtgactaaagtaaatattgtaattattgacgatctgcagttattccgtgaaggtgtcaaacggattttggatttcgagcctacctttgaagtagtggccgaaggagacgacggagatgaagcggctcgcattgtcgagcactaccatcctgatgttgttatcatggatattaatatgccgaatgtgaacggagtagaagcgacaaaacaactggtcgacttgtatccggaatcaaaggttattattttatccatccatgatgacgaaaactatgttacacatgcattaaaaacaggagccctcaggagacc

SEQ ID NO: 33 - DNA, 24 bp, Artificial Sequence
  Oligonucleotide Protospacer degU target gene
tacgggatttcgaacctacctttg

SEQ ID NO: 34 - DNA, 24 bp, Artificial Sequence
  Oligonucleotide Protospacer degU target gene
aaaccaaaggtaggttcgaaatcc

SEQ ID NO: 35 - DNA, 43 bp, Artificial Sequence
  Oligonucleotide HomA eps operon
gaggaggcagtcaagcatgtgacaggcggtttttttgctatgc

SEQ ID NO: 36 - DNA, 35 bp, Artificial Sequence
  Oligonucleotide HomA eps operon
ggtggtctctaccctgatcggggaatatcggagtc

SEQ ID NO: 37 - DNA, 35 bp, Artificial Sequence
  Oligonucleotide HomB eps operon
ggtggtctcttgagcctgtacaagatgggttctcc

SEQ ID NO: 38 - DNA, 43 bp, Artificial Sequence
  Oligonucleotide HomB eps operon
gcatagcaaaaaaaccgcctgtcacatgcttgactgcctcctc

SEQ ID NO: 39 - DNA, 24 bp, Artificial Sequence
  Oligonucleotide Spacer eps
tacggcattcggacggtagcccgt

SEQ ID NO: 40 - DNA, 24 bp, Artificial Sequence
  Oligonucleotide Spacer eps
aaacacgggctaccgtccgaatgc

SEQ ID NO: 41 - DNA, 524 bp, Artificial Sequence
  5-prime region of the CAT gene with flanking BsaI sites
ggtctcagagaacctcatcctgtattgtaaatgaattgtagcgcgtttcggaggccgaagcaaccgatccctccttctttgtattcttgctgtgccgttctccgtctctcacccccggctgtcatttaggttatactgacatatatcggggtgaagatatgaacagcaaatcacgattgacgcctgaaatgtggcgggcaattacggaaaacgattccgcctatgacggagttttttattacgcggtcaaaacgaccggcatattttgccgcccatcctgcaaatcgagagttccgcaaatcgacaatgtgcagatttttttcaatgcaaaagatgctttatcagaagggttccgcccctgcaaacgctgcaatcccgccggagcgctgctgccggatgaagagctggcacagcgggtagtgcaaatcatcgagaaatcttatcgcgatacgctgtctctgcaagctttggctgacaggtgccatatcagcccttttcacctgcagcggacatctcatgagacc

SEQ ID NO: 42 - DNA, 424 bp, Artificial Sequence
  3-prime region of the CAT gene with flanking BsaI sites
ggtctcaacccaatcctatgtataccggcttttcactccggcttccggcagggtaaataaaaggattggagtgatcaagcaatgccgcaaaacaataacaatcgatatgaactctttttcaaaaaaagatacaaagttctacatatattaaacgattttctcatcggccttttattccttgtcgggagcttctgctttttctccgaggaactgaagccggcgggactatggatgtttgtgatcggcagctttcagctgttaatcaggccgacgatccgtttggttcatgattttcactacagaaagtttgttgagagccggcgttccaaatgccgggagtaaacacaccacagtaagaccgctaagcagcgcatataaaaaaggccgaatcatatcggcctttcctctgtcatgcgacgagacc

SEQ ID NO: 43 - DNA, 34 bp, Artificial Sequence
  Oligonucleotide HomA slrA
ggtggtctctacccttccacttaatgctgatcgg

SEQ ID NO: 44 - DNA, 39 bp, Artificial Sequence
  Oligonucleotide HomA slrA
acgcttaggatcccatagcttcctccaatatgataagac

SEQ ID NO: 45 - DNA, 57 bp, Artificial Sequence
  Oligonucleotide HomB slrA
tcttatcatattggaggaagctatgggatcctaagcgtttaaaagcatcagttatcc

SEQ ID NO: 46 - DNA, 38 bp, Artificial Sequence
  Oligonucleotide HomB slrA
ggtggtctcttgagcagtttaagataacaacaaccatc

SEQ ID NO: 47 - DNA, 24 bp, Artificial Sequence
  Oligonucleotide Spacer slrA
tacgagattgggtgattttgatga

SEQ ID NO: 48 - DNA, 24 bp, Artificial Sequence
  Oligonucleotide Spacer slrA
aaactcatcaaaatcacccaatct

SEQ ID NO: 49 - DNA, 40 bp, Artificial Sequence
  Oligonucleotide HomA tapA-sipW-tasA
ggtggtggtctctacccgaagtcattcaataaatcctttc

SEQ ID NO: 50 - DNA, 39 bp, Artificial Sequence
  Oligonucleotide HomA tapA-sipW-tasA
gctaggagggtagtctctgtggtgcaatttaaaaaacgg

SEQ ID NO: 51 - DNA, 35 bp, Artificial Sequence
  Oligonucleotide HomB tapA-sipW-tasA
attgcaccacagagactaccctcctagcgagtatg

SEQ ID NO: 52 - DNA, 37 bp, Artificial Sequence
  Oligonucleotide HomB tapA-sipW-tasA
ggtggtggtctcttgagctcaatgtgacatggtattg

SEQ ID NO: 53 - DNA, 24 bp, Artificial Sequence
  Oligonucleotide Spacer tasA
tacggctaaagcaattgaagccgc

SEQ ID NO: 54 - DNA, 24 bp, Artificial Sequence
  Oligonucleotide Spacer tasA
aaacgcggcttcaattgctttagc

SEQ ID NO: 55 - DNA, 794 bp, Artificial Sequence
  Gene synthesis construct
ggtggtctctacccaagtcgcactgtttgcagaccgttcggatatcacggaagaagtgacgaggctgaaaagccatttccgccagttccgcgatatttgcaaagcgggaggagccgcggggagaaagctcgatttcctcgtccaggagctcaaccgtgaagcgaacacgatcggttcaaaagcgaatgatcaccagatcacaaaacatgtggtcgaaatgaaaagctctattgaaaaaataaaagaacaagtgcaaaatatagaatagcgattgtgcgtattgtttacggatgttctctgcaggttaaactagagacgtccaagtacagggggaacgtataggatgacgattaaactgatcaatatcggctttggaaatatcatatccgcgaattggctgatctcgattgtgagtcctgagtccgcatcgattaagcggatgatccaggatgcccgcgacagaggcatgcttatagatgctacatatggaagaagaacccgtgcggttgtcattatggacagtgaccatatcatcttatctgccgtccagcctgagacagtagcacaaaggctttccgttaaagaagaaattatggatgaagggcagggataagagctttatgaaagaaagaggtttgttaatcgttctctccggcccttccggcgtcggaaaaggaacagtcaggcaggcgctgtttgctcaggaggacacaaaatttgaatattcgatttcggtgacgacaagaaaaccgcgtcaaggcgaaagagacggcgtcgactctcaagagaccacc

SEQ ID NO: 56 - DNA, 24 bp, Artificial Sequence
  Oligonucleotide
tacgtcctggatcatccgcttaat

SEQ ID NO: 57 - DNA, 24 bp, Artificial Sequence
  Oligonucleotide
aaacattaagcggatgatccagga

SEQ ID NO: 58 - DNA, 110 bp, Artificial Sequence
  Synthetic promoter Psy flanked by type-II restriction sites
atatatgaagactttcgagagtaataagtatcttcatatctattgacaactgaagatatattctatataatcgttctagaaaaggaggtagaatatttgtcttcatatat

SEQ ID NO: 59 - DNA, 218 bp, Bacillus licheniformis
  misc_feature (1)..(218): Functional fragment degQ gene with 3-prime region flanked by type-II restriction sites
gaagacctatatggaaaagcaacaaattgaagaattaaaacaactgctttggcggctagagaatgaaatcagagaaacaaaggactccttgcgcaagattaacaaaagcattgatcaatacgataagtacacatatctaaaaacctcgtaaaaagacttgtttcagacaagtcttttttttgtgtgcaaacgaacccaaaaaaaggtagcaagtcttc

SEQ ID NO: 60 - DNA, 173 bp, Bacillus subtilis
  misc_feature (1)..(173): Native 5-prime region comprising P1 degSU promoter
attcgtacagtctttagaatttttgtgcgtattttggtatcataaagagtagatagtatataaaaatgtttttttctagaatatacgcattctttcattataattcgacataatttgcagatcaattacatttataataaaaatatatgacaacgccgtgacggagggaaatt

SEQ ID NO: 61 - DNA, 136 bp, Bacillus subtilis
  misc_feature (1)..(136): Native 5-prime region comprising P2 and P3 promoters of degU
tcgaaaataggtcttgggacatttattatgattaaggttccgttatctctttgactatgatttgtaaaatagagccaaaaggcatattgaccgaatgctagagtatatagaacaataatacaaggaggcgtggctt

SEQ ID NO: 62 - DNA, 172 bp, Bacillus licheniformis
  misc_feature (1)..(172): Native 5-prime region comprising P1 degSU promoter
aatcgaacagacttagaattttggcgcgtattttgttatgattaaatagaaatggatcggtaaccataaaaaaaccagaatacgcattctttcattataattcgacacgaatttcagttcaatgaaatttatgataaaactaaatgacaatgctgttacggagggaaaatgg

SEQ ID NO: 63 - DNA, 135 bp, Bacillus licheniformis
  misc_feature (1)..(135): Native 5-prime region comprising P2 and P3 promoters of degU
tcgaaaataggtcttgggacatttatattgattaaagttccactgtctttgtaaagataattgtaaaatagagacaaaagacatattgaccataaaagcggtgtgtttaacaatgagaatggggaggcgtagctt

SEQ ID NO: 64 - DNA, 546 bp, Bacillus subtilis
  misc_feature (1)..(546): DNA encoding bslA
atgaaacgcaaattattatcttctttggcaattagtgcattaagtctcgggttactcgtttctgcacctacagcttctttcgcggctgaatctacatcaactaaagctcatactgaatccactatgagaacacagtctacagcttcattgttcgcaacaatcactggcgccagcaaaacggaatggtctttctcagatatcgaattgacttaccgtccaaacacgcttctcagccttggcgttatggagtttacattgccaagcggatttactgcaaacacgaaagacacattgaacggaaatgccttgcgtacaacacagatcctcaataacgggaaaacagtaagagttcctttggcacttgatttgttaggagctggcgaattcaaattaaaactgaataacaaaacacttcctgccgctggtacatatactttccgtgcggagaataaatcattaagcatcggaaataaattttacgcagaagccagcattgacgtggctaagcgcagcactcctccgactcagccttgcggttgcaactaa

SEQ ID NO: 65 - DNA, 705 bp, Bacillus subtilis
  misc_feature (1)..(705): DNA encoding epsA
atgaatgagaatatgagtttcaaagaattatatgcgattgtcagacacagattcgtgctgattctgctcatcacaatcggcgtcacccttattatgggttttgtgcaatttaaggtcatttcaccgacctaccaggcgtcgacacaggtgctggttcatgaatcagacggtgaagaaaactcgaatctcagtgacatccagcgaaatcttcagtatagcagcacgttccaatcgattatgaaaagcactgccttgatggaagaagttaaggcggaattgcacctatctgaatcggcttcctcgctgaaaggaaaagtggttaccagcagtgaaaatgaatcagaaataatcaacgttgccgttcaggatcacgatccggcgaaagcagctgagattgcgaacacgttagtgaacaagtttgaaaaagaagtagatgaaagaatgaatgtacaaggcgtacatatattatcagaggcgaaggcttcggaaagcccgatgatcaagccggccaggctgcgaaatatggtcatggcttttggcgctgctgtcatgggcggcattacactggcattttttctgcattttctcgatgatacatgcaaaagcgcacggcagctcagcgagagaaccggattgccatgcttaggctccgttcctgatgtccacaaagggcggaatcgcgggataaaacatttcggggagtga

SEQ ID NO: 66 - DNA, 684 bp, Bacillus subtilis
  misc_feature (1)..(684): DNA encoding epsB
gtgatctttagaaaaaagaaagcaaggcgaggtttggctcaaatatctgttttacacaataaatcagttgttgcggaacaatatcgcaccattcggacaaacattgagttctcatctgtccagaccaacttgcgatctatcctcgtcacctcctctgtgcctggtgaaggtaaatcgttcagtgcagcgaatcttgcggctgtctttgcgcagcagcaggaaaagaaagtactgctggtggatgccgatttaagaaagccgaccatcaatcagacgtttcaggttgataatgtaaccgggctgacaaatgtgctggtcggcaatgcttcactcagtgagacggtgcaaaagacgccgatcgataacttatatgtactgacaagcgggccgaccccgccaaacccggcagaactgttgtcttcaaaagcaatgggagatttaatatctgagatctatgaacaattcagcctcgtcatctttgattcccctcctcttttggctgttgcagatgctcagattctagcaaatcagacagacggcagcgtgctcgtcgttttaagcggaaaaacaaaaaccgataccgttctgaaagcaaaagatgcactggaacaatccaatgcgaagctgttaggcgctcttttaaacaaaaagaaaatgaaaaaatcggaacactattcctactag

SEQ ID NO: 67 - DNA, 1797 bp, Bacillus subtilis
  misc_feature (1)..(1797): DNA encoding epsC
atgattattgcgctggatacttacctcgttttaaattcagttattgcaggatatcaatttttaaaagattcctatcaattttatgactccggagcattactgcttaccgctgtcagcttgctcctcagctatcatgtgtgtgctttcctgttcaatcagtataaacaggtgtggacatacaccgggcttggcgagctgattgtcctgcttaagggcattacgctttcagccgctgtgaccggcgtcattcagtatgctgtgtatcacacgatgttcttccgtctgttaaccgcgtgctgggtgcttcagcttttgtctattggagggacccgtattttatccagagtattaaacgaaagcatcaggaaaaaacgctgcgcctcgtcccgcgcgctgattatcggggcgggctcaggtgggactctgatggtcaggcagctgctttcgaaagatgaacctgatatcatacctgtcgcttttattgatgacgaccaaacgaagcataaattagaaattatggggctgcccgtaatcggcggaaaagaaagtatcatgcctgcggtgcaaaagctcaaaattaattatattattattgccattccttcactccgcacccatgagcttcaggtgttatataaagaatgtgtgcgaaccggagtaagcattaaaattatgcctcattttgatgaaatgctgcttggcacacgaactgccggacaaatcagagatgtaaaagctgaggacttgctcggcagaaagccggtaaccctcgacactagcgaaatttcgaaccgcatcaaaggaaaaacagttctcgtcacgggagcgggcggatcaatcggctcggaaatctgccgtcagatcagcgcgtttcagcctaaggaaatcattctgctcggccatggggaaaacagcattcattcgatttatacagagctgaacggacgattcggcaaacacattgtgttccatacggaaatcgctgatgtgcaggaccgcgataaaatgtttaccttgatgaaaaaatacgagccgcatgttgtctatcatgcagctgcccataagcatgtgcctttaatggaacacaatccagaagaggcggtcaaaaacaatattatcggaacaaaaaatgtcgcggaagcagccgatatgtcgggaactgagacattcgtgctgatttcatcggacaaagcggtgaacccagccaacgtaatgggggcgacaaaacgattcgcagagatgattattatgaatcttgggaaagtcagcagaaccaaatttgttgctgttcgcttcggcaatgtactcgggagccgcggcagcgtcattccaattttcaaaaaacagattgaaaaaggcggcccggtgacagtaacacatccggcaatgacccgctatttcatgacgattcccgaggcatcaaggcttgtgattcaggctggggcactggcgaaagggcgtcaaattttcgttctcgatatgggagagcccgtaaagattgtggatcttgccaaaaacctcattcatttgtccggctacacgactgagcaggttccaatcgaattcacaggcattcgtccgggcgaaaaaatgtatgaagaattgctgaacaaaaatgaagtccatgctgaacaaatctttccaaaaattcacatcggtaaagcggtggacggcgattggccggtgctgatgcgctttatcgaggattttcatgagctgccggaagccgacctgagagcgaggctgtttgcggcaatcaatacatcagaagaaatgacggctgccagcgttcattag

SEQ ID NO: 68 - DNA, 1146 bp, Bacillus subtilis
  misc_feature (1)..(1146): DNA encoding epsD
atgacgaaaaagatattgttttgcgcgactgttgattatcattttaaggcctttcacctcccttattttaaatggttcaagcaaatgggctgggaggttcatgtcgccgcgaacggacaaaccaagctgccgtatgtggatgagaaattctccatcccgattcgcaggtcaccttttgaccctcagaacctggccgtttataggcagctgaagaaagtgattgacacttatgaatacgacattgtccattgccatacaccggtcggcggcgttctcgccagactggcggcgaggcaggcacggcggcacggaacaaaggtgctgtacacagcgcacggatttcacttctgcaaaggggcaccgatgaaaaattggcttctttactatccggttgagaaatggctttcagcatatacagactgcctgattacgattaatgaagaggattacatacgggcgaaaggacttcaaaggccgggcggaaggacgcagaaaattcacggcattggcgtcaataccgagcgtttccggcctgtcagtccgatagagcagcaaagactcagagaaaagcacgggttccgtgaagatgattttatattggtttatccggctgagctcaatctgaacaaaaaccagaagcagttaattgaagccgcagccttgctaaaagaaaaaattccctcactccgccttgtgtttgccggggaaggggcaatggaacatacgtatcaaacgttagctgaaaagcttggtgcctccgcccatgtctgtttttacggcttttgcagcgacatacatgagttgattcagcttgcggatgtatctgtcgcatccagcattagagaaggcctcggtatgaatgtgcttgagggaatggcggcagaacaaccggcgatcgccacagataatcgcgggcatcgggaaatcatccgcgacggagaaaacggttttctgatcaaaatcggtgacagtgctgcttttgcccgccggattgaacagctttaccataagccggagctctgccgaaagctgggacaggaaggccgaaaaacagccttgcgcttctcggaggcgcgaacggtggaagaaatggcagatatttattccgcgtacatggatatggatacaaaggagaaaagcgtatga

SEQ ID NO: 69 - DNA, 837 bp, Bacillus subtilis
  misc_feature (1)..(837): DNA encoding epsE
atgaactcaggaccgaaagtttctgtcattatgggcatttataattgcgaacgcactttggcagaaagcatagaatccatactcagccaatcctataaaaattgggagctgattttgtgcgatgatgcgtcaacagacggcacgctccgtatcgcgaagcagtatgccgctcattacagcgaccgcatcaaactgattcaaaacaaaacaaataagcggcttgccgcatcattaaatcattgtctttcgcatgcgacaggcgattatatcgcacgtcaggacggagatgacctttcgtttccgcgccgtctggaaaagcaggtcgcgtttttagaaaagcaccgacactatcaggtggttggcaccggcatgcttgtgtttgatgaatttggcgtaagaggcgcccgcattctgccttctgttccggagccgggcatcatggcaaaagggactccattttgccacggcacgattatgatgagagcgagtgcctaccgcacgctgaaaggctaccggtcggtgcggcggacgagacgaatggaagatattgatttgtggcttcgcttttttgaagagggcttcaggggctataatcttcaggaagccttgtataaagtgagggaagacagcgatgcattcaaacggcggtcatttacgtattcaatcgacaatgccattcttgtctatcaggcgtgcagacgcttgaagcttcctttatctgattacatatatatcgcaaaaccgttaattcgcgcctttatgcctgcagctgtgatgaatcgctaccataaaaaaagagtgatgaaccaaaaggaagggcttgtcaagcatgaatag

SEQ ID NO: 70 - DNA, 1155 bp, Bacillus subtilis
  misc_feature (1)..(1155): DNA encoding epsF
atgaatagcagccaaaagcgcgtgctccatgttctcagcggcatgaacaggggcggcgcggaaaccatggtaatgaatttatatcggaagatggacaaaagcaaagtgcaatttgattttttaacgtatcgaaatgatccgtgcgcttatgatgaagagattttatctttaggcgggcggcttttttatgtcccgagcattgggcaaagcaatccccttacatttgtgaggaatgtgagaaacgcgataaaagaaaatgggccgttcagcgccgttcatgcgcacacggatttccaaacgggttttatcgcccttgcggcaaggctcgccggagtgccggtcagggtatgccactcccacaatacgtcttggaagaccggcttcaactggaaggatcgattgcagctgctcgtgttcaggcggctcattttggcaaatgcgacagcgctgtgtgcctgcggagaggatgcgggcaggtttttatttggacagtccaatatggagcgggagcgtgttcaccttcttcctaacgggattgaccttgagttgttcgccccaaatgggcaggcggctgatgaagaaaaagcagcacgcggcattgcagccgaccggctcatcattggccatgtggcccggtttcatgaagtgaaaaaccacgcgttcctgttgaagcttgccgcacatctcaaggaaagaggcattcgctttcagctcgttctggcgggagacgggccgttgtgcggggagatagaggaggaggcgcggcagcagaatttgctatcagacgtcctctttttaggcacggaagaacggatccatgaactgatgcgaacattcgatgtatttgtcatgccgtctctgtacgaaggcttgccggttgtgcttgtggaagcgcaggcgtcggggcttccatgcatcatttcagacagcattacagaaaaagtcgacgccggtctcgggcttgtcacaagattaagtctttctgagccgatcagcgtctgggctgaaaccattgcaagggcggccgccgcaggcaggccgaagcgtgagttcatcaaagaaacactcgctcaacttggctacgatgcacagcaaaatgtaggagcgctgctgaatgtatacaacatcagcacggaaaaggaccataaccgatga

SEQ ID NO: 71 - DNA, 1104 bp, Bacillus subtilis
  misc_feature (1)..(1104): DNA encoding epsG
atgattgtatatgccgtcaatatggggattgtatttatttggtcttggttcgctaaaatgtgcggcggccgtgatgattcgcttgccacggggtatcggccgaataagcttttgatctggattccgctcgcttcacttgtgctcgtgtcaggtctccgctatcgagtcggcacggattttcagacgtacacgctgttgtacgaattggcgggcgattatcaaaatgtgtggcagatattcggtttcggcacagcgaaaacagcgacagatccggggtttaccgcactcctttggctgatgaatttcatcacggaagatcctcaaatcatgtattttacggtggcggtcgtgacctacagctttattatgaagacactcgccgactatggcaggccgtttgagctgagtgtctttttatttttgggaacctttcattattacgcatcttttaacggcatcaggcaatacatggtggcagctgttttgttttgggcgatccgttatatcattagcgggaactggaagcgatatttcctgattgtgctggtcagctcgctctttcattcgtcggcgctgattatgattccagtgtactttattgtcagaagaaaagcctggtcaccggcgatattcggcctatccgctttatttctcggcatgacatttttatatcaaaaatttatttctgtgtttgtcgttgtacttgaaaacagctcatacagccattatgaaaaatggctcatgacgaacacaaatggaatgaatgtgatcaaaatcgctgttttggttctgccgctgttccttgcattttgctataaagaacgactgcggagtctgtggccgcaaattgatattgtcgtcaatttgtgcctgctaggttttttgttcggccttttggccacaaaggacgtgatttttgccagattcaatatttatttcggtctgtatcaaatgatcctagtcccttatttcgtcaggatatttgatgaaaaatcgaacgctcttatctatatcgctatcgttgtttgttattttctttacagttatttgcttatgccggtcgattcatcggttctgccttacagaacgattttttcccggtaa

SEQ ID NO: 72 - DNA, 1035 bp, Bacillus subtilis
  misc_feature (1)..(1035): DNA encoding epsH
atggaaacacctgcggttagtctgttagtcgctgtttataacacagaaacatatatcagaacgtgtctcgaatcactgcggaaccagacaatggacaatattgaaatcatcattgtcaatgacggttcagctgacgccagcccggatatcgcagaagaatacgccaaaatggacaacaggttcaaggtgattcatcaggaaaaccagggactcggtgcggttcggaataaaggcattgaagctgcacgcggcgaatttatcgcgtttatcgattcagacgattggattgagcctgattattgcgagcagatgctccggacagcaggcgatgaaactgatctggtcatttgcaattacgccgcagagtttgaggacactggcaaaaccatggactctgacattgcccaaacctatcaggatcagccgaaggagcactatatcaaggcgttattcgaagggaaggtcagagggttttcatggaacaaactgtacagaagaagcatgattgaagcccatcggctgtcgtttccgctccgaggcgagctggagcatgtcgaggatcagtttttcagcttcagggctcattttttcgcccgctcagtatcctacgtaaaaacgccgctctatcattatcgaattcacctttcctccattgtgcagcgctatcagaaaaaattgtttgaatcaggacttgcgctgtatgagacgaatgcggcgtttttacaggagaacaacaaactggaggagtatcgcaaggagcttgatacctttatcgttcttcacagcagcatctgtatgctgaatgaatggaaaacgagcggcagccgccggctgtttgaaaagcttagaaatgttggcgtgatatgcgcggacccggtgtttcaagaaagtctttcaaaaacgggtactgctccttttgacgcaaaacggtcatgcctgcttctgatggcgaaatacagaatgattccgttcgtcgctatggcatcggctgtgtatcagcgggtgatcgagtacaaaatgagaaacagagggtga

SEQ ID NO: 73 - DNA, 1077 bp, Bacillus subtilis
  misc_feature (1)..(1077): DNA encoding epsI
atgtcgttacaatcgttgaaaatcaattttgcagaatggctgctgctaaaggtcaaatacccgtcccaatattggctgggagcggcagatcaaccggtaaaggccgcagcacatcagaaaaaaatcatactgaccctgctgccgtcccatgacaatttgggagatcacgcaattgcttatgccagcaaggcatttcttgagcaagaatacccggactttgacatcgtcgaggtcgatatgaaggacatttacaaatcagcaaaaagcctgatccgctcgcgccatccggaggatatggtctttatcatcggcggcggaaacatgggggatttataccgttatgaggagtggacgcgccgcttcatcattaaaacattccatgactatcgggttgtccagcttccggcaacggctcatttttctgacacgaaaaaagggcgcaaagagctgaaacgggcacagaaaatttataatgcgcaccccggcctattgctgatggcgcgggatgaaacaacgtatcaatttatgaaacagcattttcaagaaaaaacaattttgaagcagccggacatggtgctgtatttagacagaagcaaggctcccgcagaacgcgaaggggtttatatgtgtttgcgcgaggatcaggaaagcgtgcttcaggaggagcagaggaaccgggtcaaggctgcgctatgtgaggaattcggcgagatcaaatcctttacgacaacgatcggccgccgggtcagccgcgatacacgcgaacatgaacttgaagcactgtggtctaagctgcaaagcgcagaagccgtcgtcactgacaggcttcatggcatgattttttgcgcgctgacaggaacgccgtgtgttgtcattcgctcctttgaccataaggtgatggagggctatcaatggcttaaagacatcccgttcatgaagctgatagaacatccggagccagagcgcgtaacagccgcagtcaatgagcttttaacaaaagaaacatcccgtgcaggctttccgagagatgtgtattttaaaggtctgcgtgacaaaatcagcggtgaagcgcaatga

SEQ ID NO: 74 - DNA, 1035 bp, Bacillus subtilis
  misc_feature (1)..(1035): DNA encoding epsJ
atgatcccgctcgtcagcattattgtcccgatgtataatgttgaaccatttatagaagagtgcattgattctttgcttcgtcaaacgctttctgatattgaaatcatcctcgtgaatgacggaacaccggatcgttcaggcgaaattgcagaggactatgcaaaacgggatgcgagaatccgggtcattcatcaggcaaacggcgggcttagttcagcgcgaaatacgggaataaaggccgcgcggggcacttacatcggctttgtagacggagacgattatgtatcatccgccatgttccagaggctgactgaagaagcggagcaaaatcagcttgacatcgtcggatgcggtttttacaagcagtcatcggacaggcggacatatgtgccgccgcagcttgaggcaaaccgcgtgctgacgaaaccagaaatgactgaacagcttaaacatgctcacgaaacgagatttatctggtatgtatggcgttatctttaccgtcgtgagctttttgaaagggcgaatctgctgtttgatgaagacatccgttttgctgaagactctccctttaatttgtccgcttttcgcgaagcggagcgggtgaaaatgcttgatgaaggattgtacatttatcgtgaaaacccgaacagcctgacagaaatcccttataagccggcgatggatgaacatcttcaaaaacaatatcaggctaaaatcgcattctacaatcactacggcttagcaggcgcatgtaaagaagatttgaatgtgtacatttgcaggcaccagcttccgatgcttttggcaaatgcctgtgcttctccgaattcgccgaaagacatcaaaaagaagatcagacagattttatcctatgacatggtgcggcaggctgtcagacatacaccgtttcagcatgagaaattattaagaggagagcgtttggtattagcactgtgtaaatggcggctcacttttctcatcaagctgtttttcgagcagcgggggacaatgaaaggcagtgcgaagcaggcatga

SEQ ID NO: 75 - DNA, 1518 bp, Bacillus subtilis
  misc_feature (1)..(1518): DNA encoding epsK
atgaaattcacgataaatttcagcgcgaatctcacggcttttctcttgtctgttttcctatcggtttggatgacgccttttattgtcaaaacgctcggtgtcgaagcgtttggctttgttcacttgacacaaaatgtgattaattacttttcggttattaccgtggcgctcagctcggttgtcgttcggttcttttctgttgctgcccacaggggagagcgggagaaagcaaatgcgtatatcagcaattatttagccgcctctgttttgatttccttgctgctcttgctgccgcttgcgggttcggctttttttattgaccgcgtcatgaatgtgccgcaagcgcttttggcagatgtgcgtttgtcgattttaatcggcagtgtgctgtttattttaacgtttctgatggcgggcttcggcgctgcaccattttatgccaaccgcctttatatcaccagttccattcaggcggtgcaaatgcttatacgggtgctgtctgtgctgctcctgtttgcatgctttgcgccgaaaatctggcagatccagcttgcagctttagctggtgctgttattgcgtctgtgctgtctttctatttcttcaaaaagctgattccgtggttttcgtttcgtatgaaggatctttcattccgtacaagcaaggagctgtttcaagcgggcgcatggagctccgtcaatcaaatcggcgtcctgctttttttgcagattgatctgttaaccgccaatttgatgctgggggcgtctgcatccgggaaatacgcggcgattatccagtttccgctgcttttgcgcagcttggccggaacggtcgcatccctgtttgcgcccatcatgacttcatattattcaaaaggcgatatggaaggattgatgaattacgccaataaggcagtaaggctcaatggtcttttgcttgcacttcctgctgccttattgggcggattggcgggaccttttctgacaatctggctcggaccgtccttttcaacgatagcaccgcttttatttattcatgccggatacttggttgtcagcctcgcctttatgccgctgttttatatatggaccgcttttaatcaacaaaaaacaccggcgattgttaccctgctgttaggtgcggtgaatgtggtgctggcggtcacgctgagcgggccggctcatctcggtctgtacggcataacattggcaggggccatttctcttattttaaaaaatgccatctttacgccgctttacgtatcccgcattacaggctacaaaaagcacgtgttccttaaaggcataatcgggcctctttcagccgctgtatttgcctggacggtctgtaaggcaattcagttcattgtgaagattgacagctggccgtcattgatagcgacgggagtgacagtcagcttttgctacgctgttttcgcttttatgctcgtttgtacaaaagaggaaagacagctggtattaaaacggtttcgaaaaacgaaaggagctgtgaatctttga

SEQ ID NO: 76 - DNA, 609 bp, Bacillus subtilis
  misc_feature (1)..(609): DNA encoding epsL
ttgatcctgaaacgactttttgatctgacggccgccatttttttgttgtgctgtacaagtgtgatcattctgttcaccatcgccgttgtcaggctgaaaataggatcacctgtcttctttaagcaagtaaggccgggcctgcacggcaaaccgttcaccctatataagttccggacaatgacggatgaacgggacagtaaaggaaatctgctgcctgatgaagtccggctgacgaaaacgggcaggctgatcagaaagctgagcattgatgagcttccgcagctcctgaatgtcctgaagggcgatctgagccttgtcgggccgcggccgcttttgatggactatctgcctctttatacagaaaaacaggcacggcgccatgaggtgaagccgggtatcacaggctgggcgcaaatcaatggcagaaacgcgatttcctgggaaaagaaatttgaattagatgtttggtacgttgacaactggtcattttttctcgatttgaaaattttatgtttgacggtgcgaaaggtccttgtttcagaagggattcagcaaaccaatcatgtgaccgcggaacggtttaccggaagcggagatgtgtcctcatga

SEQ ID NO: 77 - DNA, 651 bp, Bacillus subtilis
  misc_feature (1)..(651): DNA encoding epsM
atgaaaaatgtggccattgtgggtgacggcggtcacggaaaggtgatcagagagctgataaacgcccgctcagatacgcgcttagccgcggtgctggatgataaattcaaaacgttcgaaggcggaaaagaatggtacacaggaccgccgaaagccgttactgaactgcgcaggctcattcctgatgtgctgtttctgattgctgttgggaataacagtgtcagaaaacagctggcggagcgactgggactggggaaagatgattttattacattgattcacccgtcagccatcgtcagcaagtcggctgtcattggggaagggacagtgattatggcgggcgcgatcattcaggcggatgcgcgcatcggcgcccactgcatcatcaatacgggtgcagtggcagagcacgacaatcaaatcagcgattacgttcatctgtccccgcgtgccacgctgtcaggagcggtttccgttcaggaaggcgctcacgtcggaaccggtgcatccgtcataccgcagatcataatcggggcttggagcattgtcggagccggctccgcggtgatccgttccataccggacagggtaacggcggccggtgctccggcacgcatcatttcttccattcaaacatcaaacaaaggatga

SEQ ID NO: 78 - DNA, 1167 bp, Bacillus subtilis
  misc_feature (1)..(1167): DNA encoding epsN
atgcataaaaaaatctacttatctccccctcatatgagcggcagggagcagcactatatttcagaagcctttcgctcaaactggattgcgccgcttgggccgctcgtgaattcatttgaagaacagctggcagaacgagtcggtgtaaaagcagcagctgcggtcggctcaggaacggcggcgattcatctcgcgctgcgtttgcttgaggtaaaagaaggtgacagcgtgttttgccagtccttcacatttgtagcaaccgccaatccgattctatatgaaaaagcggtgcccgtctttattgattctgagcctgatacgtggaatatgtcgccgacagcccttgaaagagcattggaggaagcgaaaagaaacggaacactgccaaaagcggtaattgccgtcaatctatatgggcagagcgcgaaaatggatgaaatcgtaagcctgtgtgatgcatacggagttcctgtcattgaggacgcagccgaatctctcggcacagtctataaagggaagcaaagcggaacattcgggcgcttcggcattttttcatttaacgggaacaaaatcatcaccacatcagggggagggatgctcgtttcaaatgatgaagccgcaattgaaaaagcaagatttctcgcttcgcaggcacgagagccggctgttcattatcagcacagtgaaattggacataattacaggctgagcaatatcttagccggcgtcggcattgcccagcttgaggtgctggatgagcgagtggagaaaagaaggaccattttcacgagatacaaaaatgcgctcggtcacttagacggcgtccgctttatgccagagtacgcagcaggcgtatccaatcgctggctcaccacgctcacacttgataacgggctcagcccatatgacatagttcaacgtcttgctgaagaaaacattgaagcccgtccgctgtggaagccgctccatacccagccgctgttcgatccggctttattttattctcatgaagatacaggaagcgtatgcgaagatttgttcaagcgaggaatctgtctcccatcggggtccaatatgacagaagatgagcaaggccgggtcattgaagtgctactgcacttattccatactgtcgaggtgaagaaatggacagcaagcattcgatga

SEQ ID NO: 79 - DNA, 969 bp, Bacillus subtilis
  misc_feature (1)..(969): DNA encoding epsO
atggacagcaagcattcgatgatcagcctgaaacagaaactgtccgggctgctcgacgtcattccgaaacaatcagagattatatatgctgactatcctctatatgggaatgtaggggatttatttattatgaaaggaacagaagccttctttaaagaacacgggattcgggtcagaaaacgctggaatccagacaatttcccaatcgggcgaaagcttgatccgaatctcatcatcgtctgccagggaggcggaaacttcggggatctgtatccgtattatcaaggctttagagagaaaatcgtccaaacctatccgaaccacaaaattgtgatcctgccgcaatcgatttattttcaaaacaaagacaacctcaagcggacggcagagatattttctaagcatgcgaaccttcacatcatgacaagggaaaaagcctcctatgctacggcacaggcctattttacaacaaatcacattcagcttctgcctgatatggctcatcagctgtttcccgtcattcccacgcagcagccgtccaatcaaaagctgagatttatccgaacagatcatgaagcaaaccaggcgcttcaggaacacgcagaagcggaaagctacgactggcgcacggtgctgtcagcttcagaccgccggacgattgcttttctccaaacgctgaacgtcttgaataaaaaagcaggcaaccctttgcccattgcgtatatatgggaaaaatactcggattatatcgtccaaaaagcgattcggtttttcagccgttacgaatcggtggaaacatcaaggctgcacggccacatcctgtcttctcttcttcaaaaagaaaacacggtcattgataattcctacgggaaaaacgccaattactttcatacctggatggaaggcgtgccaagcacccgtctcatccagcacgcctcaaagaaggaaaaccttcctgctcacatgtga

SEQ ID NO: 80 - DNA, 762 bp, Bacillus subtilis
  misc_feature (1)..(762): DNA encoding tapA
atgtttcgattgtttcacaatcagcaaaaggcgaagacgaaactgaaagttctgcttatctttcagctttcagtcattttcagtctgactgccgcaatatgcttacaattttccgatgatacaagcgctgcttttcatgatattgaaacatttgatgtctcacttcaaacgtgtaaagactttcagcatacagataaaaactgccattatgataaacgctgggatcaaagtgatttgcacatatcagatcaaacggatacgaaaggcactgtatgctcacctttcgccttatttgctgtgctcgaaaatacaggtgagaaacttaagaaatcaaagtggaagtgggagcttcataagcttgaaaatgcccgcaaaccgttaaaggatgggaacgtgatcgaaaaaggatttgtctccaatcaaatcggcgattcactttataaaattgagaccaagaaaaaaatgaaacccggcatttatgcatttaaagtatataaaccggcaggctacccggcaaacggcagtacatttgagtggtcggagcctatgaggcttgcaaaatgcgatgaaaaaccgacagtccctaaaaaagaaacaaagtcggacgtcaaaaaggagaatgaaacaacacaaaaagatataccggaaaaaacaatgaaagaagaaacatctcaagaagctgtaaccaaagaaaaagaaactcaatcagaccagaaggaaagcggggaagaggatgaaaaaagcaatgaagctgatcagtaa

SEQ ID NO: 81 - DNA, 573 bp, Bacillus subtilis
  misc_feature (1)..(573)
atgaagctgatcagtaatattttatacgtgatcatctttactcttattattgtgctgacacttgtcgtgatttcaacacgttcatccgggggagagccggcagtgtttgggtatacgctgaaatcagttctgtcaggttcgatggagccggagttcaatacaggttccttaatattggtcaaagaaatcactgatgtgaaagagctccaaaaaggtgacgttattacatttatgcaggatgcaaatacggcggtcacccacagaattgttgacataacaaagcaaggagaccatttgttatttaaaacaaaaggtgataataatgcagcagctgattcagcgcctgtatcggacgaaaatgttcgcgcgcaatacacaggttttcagcttccatatgccggctatatgcttcattttgccagccagccgattggaacggctgtattattgattgttcccggcgtgatgctgttagtttacgcttttgtgacgatcagcagcgccattagagaaattgaaagaaagacaaaagccttggaaacagatacaaaggacagcaccatgtctacttaa

SEQ ID NO: 82 - DNA, 786 bp, Bacillus subtilis
  misc_feature (1)..(786): DNA encoding tasA
atgggtatgaaaaagaaattgagtttaggagttgcttctgcagcactaggattagctttagttggaggaggaacatgggcagcatttaacgacattaaatcaaaggatgctacttttgcatcaggtacgcttgatttatctgctaaagagaattcagcgagtgtgaacttatcaaatctaaagccgggagataagttgacaaaggatttccaatttgaaaataacggatcacttgcgatcaaagaagttctaatggcgcttaattatggagattttaaagcaaacggcggcagcaatacatctccagaagatttcctcagccagtttgaagtgacattgttgacagttggaaaagagggcggcaatggctacccgaaaaacattattttagatgatgcgaaccttaaagacttgtatttgatgtctgctaaaaatgatgcagcggctgctgaaaaaatcaaaaaacaaattgaccctaaattcttaaatgcaagcggtaaagtcaatgtagcaacaattgatggtaaaaccgctcctgaatatgatggtgttccaaaaacaccaactgacttcgatcaggttcaaatggaaatccaattcaaggatgataaaacaaaagatgaaaaagggcttatggttcaaaataaatatcaaggcaactccattaagcttcaattctcattcgaagctacacagtggaacggcttgacaatcaaaaaggaccatactgataaagatggttacgtgaaagaaaatgaaaaagcgcatagcgaggataaaaattaa

SEQ ID NO: 83 - DNA, 117 bp, Bacillus subtilis
  misc_feature (1)..(117): DNA encoding phrG
atgaaaagatttctgattggcgcaggcgtcgcagcggtgattttatcaggttggtttattgcggaccatcaaacccactcacaggaaatgaaagtcgctgagaaaatgattggataa

SEQ ID NO: 84 - DNA, 1098 bp, Bacillus subtilis
  misc_feature (1)..(1098): DNA encoding rapG
atgaataagatcgcacccgcagaaatcgctagcatgctcaacgattggtaccttgccatcaagaaacatgaagttgaagaatcctcccgtttatttgaagaagtgaagcctttattggatgacatggaagaggatcaggaggtgcttgcctacttctccttattggaactgcgccacaaggttttgcttcacgaggcgagaggacagggctttcagcatgaggagccttctcatatgaatgctacgtctgacatgctgaaatattacttttttctgtttgaaggcatgtatgaggcctataaaaataattatgacattgccattgggctgtataaagatgcagagcagtatctcgacaacattcccgatccgattgaaaaagccgaatttcacctgaaggtcggtaagctctattataagctgggacaaaatattgtgtccctcaatcatacacggcaagcagtcaaaacattcagagaagagacagattataaaaagaagctggcttcagccctgattaccatgtcaggcaattttacagagatgagccagtttgaagaagctgaggcttatttggacgaagcaattcggatcacgagtgaattagaggatcatttttttgaagcccagcttttgcataacttcggccttctacatgcgcaaagcggcaaatcagaagaagcggtttcgaaattagaggaggctctacagaacgatgagtatgcccgctccgcctattattatcattctgcctacttgctgatacgagagctgtttaagatcaaaaagaaagaacaggccttatcttattaccaagacgtgaaggaaaaattgactgctgagccgaatagaatatgtgaggcaaaaatagacattttatatgccatttatgcagaagggggtcatgcggaaacgtttcacttatgcaaacaacatatggatgacttgttgtccgagaaagagtatgacagtgtaagagaactttccattttggctggcgaacggtatagggaacttgagctttacaaagaagctgcccactttttttatgaagcattacagattgaagaactgattaaacgaacggaggttatataa

SEQ ID NO: 85 - DNA, 270 bp, Bacillus subtilis
  misc_feature (1)..(270): DNA encoding remA
atgacgattaaactgattaatatcggatttggcaatatcatctccgccaatcggatgatttcgattgtcagcccggagtctgcgccaatcaaacggatgattcaggatgcaagagaccgcggaatgctaattgacgctacatacggacgaagaacccgtgcagttgtcgtcatggatagtgatcacattatcttatctgccgtccagcctgagacagttgcacacagactttctgttaaagaagaaattatggatgaagggcaggggtaa

SEQ ID NO: 86 - DNA, 159 bp, Bacillus subtilis
  misc_feature (1)..(159): DNA encoding slrA
atgaaaactcatgttaaaaaagatttggacaaaggttggcatatgttaattcaagaagctagatccattggattaggcattcatgatgtgaggcagtttttagaatctgagacagcatcaagaaagaaaaaccacaaaaaaaccgtccggcaagactag

SEQ ID NO: 87 - protein, 181 aa, Bacillus subtilis
  MISC_FEATURE (1)..(181): bslA
MKRKLLSSLAISALSLGLLVSAPTASFAAESTSTKAHTESTMRTQSTASLFATITGASKTEWSFSDIELTYRPNTLLSLGVMEFTLPSGFTANTKDTLNGNALRTTQILNNGKTVRVPLALDLLGAGEFKLKLNNKTLPAAGTYTFRAENKSLSIGNKFYAEASIDVAKRSTPPTQPCGCN

SEQ ID NO: 88 - protein, 234 aa, Bacillus subtilis
  MISC_FEATURE (1)..(234): epsA
MNENMSFKELYAIVRHRFVLILLITIGVTLIMGFVQFKVISPTYQASTQVLVHESDGEENSNLSDIQRNLQYSSTFQSIMKSTALMEEVKAELHLSESASSLKGKVVTSSENESEIINVAVQDHDPAKAAEIANTLVNKFEKEVDERMNVQGVHILSEAKASESPMIKPARLRNMVMAFGAAVMGGITLAFFLHFLDDTCKSARQLSERTGLPCLGSVPDVHKGRNRGIKHFGE

SEQ ID NO: 89 - protein, 227 aa, Bacillus subtilis
  MISC_FEATURE (1)..(227): epsB
MIFRKKKARRGLAQISVLHNKSVVAEQYRTIRTNIEFSSVQTNLRSILVTSSVPGEGKSFSAANLAAVFAQQQEKKVLLVDADLRKPTINQTFQVDNVTGLTNVLVGNASLSETVQKTPIDNLYVLTSGPTPPNPAELLSSKAMGDLISEIYEQFSLVIFDSPPLLAVADAQILANQTDGSVLVVLSGKTKTDTVLKAKDALEQSNAKLLGALLNKKKMKKSEHYSY

SEQ ID NO: 90 - protein, 598 aa, Bacillus subtilis
  MISC_FEATURE (1)..(598): epsC
MIIALDTYLVLNSVIAGYQFLKDSYQFYDSGALLLTAVSLLLSYHVCAFLFNQYKQVWTYTGLGELIVLLKGITLSAAVTGVIQYAVYHTMFFRLLTACWVLQLLSIGGTRILSRVLNESIRKKRCASSRALIIGAGSGGTLMVRQLLSKDEPDIIPVAFIDDDQTKHKLEIMGLPVIGGKESIMPAVQKLKINYIIIAIPSLRTHELQVLYKECVRTGVSIKIMPHFDEMLLGTRTAGQIRDVKAEDLLGRKPVTLDTSEISNRIKGKTVLVTGAGGSIGSEICRQISAFQPKEIILLGHGENSIHSIYTELNGRFGKHIVFHTEIADVQDRDKMFTLMKKYEPHVVYHAAAHKHVPLMEHNPEEAVKNNIIGTKNVAEAADMSGTETFVLISSDKAVNPANVMGATKRFAEMIIMNLGKVSRTKFVAVRFGNVLGSRGSVIPIFKKQIEKGGPVTVTHPAMTRYFMTIPEASRLVIQAGALAKGRQIFVLDMGEPVKIVDLAKNLIHLSGYTTEQVPIEFTGIRPGEKMYEELLNKNEVHAEQIFPKIHIGKAVDGDWPVLMRFIEDFHELPEADLRARLFAAINTSEEMTAASVH

SEQ ID NO: 91 - protein, 381 aa, Bacillus subtilis
  MISC_FEATURE (1)..(381): epsD
MTKKILFCATVDYHFKAFHLPYFKWFKQMGWEVHVAANGQTKLPYVDEKFSIPIRRSPFDPQNLAVYRQLKKVIDTYEYDIVHCHTPVGGVLARLAARQARRHGTKVLYTAHGFHFCKGAPMKNWLLYYPVEKWLSAYTDCLITINEEDYIRAKGLQRPGGRTQKIHGIGVNTERFRPVSPIEQQRLREKHGFREDDFILVYPAELNLNKNQKQLIEAAALLKEKIPSLRLVFAGEGAMEHTYQTLAEKLGASAHVCFYGFCSDIHELIQLADVSVASSIREGLGMNVLEGMAAEQPAIATDNRGHREIIRDGENGFLIKIGDSAAFARRIEQLYHKPELCRKLGQEGRKTALRFSEARTVEEMADIYSAYMDMDTKEKSV

SEQ ID NO: 92 - protein, 278 aa, Bacillus subtilis
  MISC_FEATURE (1)..(278): epsE
MNSGPKVSVIMGIYNCERTLAESIESILSQSYKNWELILCDDASTDGTLRIAKQYAAHYSDRIKLIQNKTNKRLAASLNHCLSHATGDYIARQDGDDLSFPRRLEKQVAFLEKHRHYQVVGTGMLVFDEFGVRGARILPSVPEPGIMAKGTPFCHGTIMMRASAYRTLKGYRSVRRTRRMEDIDLWLRFFEEGFRGYNLQEALYKVREDSDAFKRRSFTYSIDNAILVYQACRRLKLPLSDYIYIAKPLIRAFMPAAVMNRYHKKRVMNQKEGLVKHE

SEQ ID NO: 93 - protein, 384 aa, Bacillus subtilis
  MISC_FEATURE (1)..(384): epsF
MNSSQKRVLHVLSGMNRGGAETMVMNLYRKMDKSKVQFDFLTYRNDPCAYDEEILSLGGRLFYVPSIGQSNPLTFVRNVRNAIKENGPFSAVHAHTDFQTGFIALAARLAGVPVRVCHSHNTSWKTGFNWKDRLQLLVFRRLILANATALCACGEDAGRFLFGQSNMERERVHLLPNGIDLELFAPNGQAADEEKAARGIAADRLIIGHVARFHEVKNHAFLLKLAAHLKERGIRFQLVLAGDGPLCGEIEEEARQQNLLSDVLFLGTEERIHELMRTFDVFVMPSLYEGLPVVLVEAQASGLPCIISDSITEKVDAGLGLVTRLSLSEPISVWAETIARAAAAGRPKREFIKETLAQLGYDAQQNVGALLNVYNISTEKDHNR

SEQ ID NO: 94 - protein, 367 aa, Bacillus subtilis
  MISC_FEATURE (1)..(367): epsG
MIVYAVNMGIVFIWSWFAKMCGGRDDSLATGYRPNKLLIWIPLASLVLVSGLRYRVGTDFQTYTLLYELAGDYQNVWQIFGFGTAKTATDPGFTALLWLMNFITEDPQIMYFTVAVVTYSFIMKTLADYGRPFELSVFLFLGTFHYYASFNGIRQYMVAAVLFWAIRYIISGNWKRYFLIVLVSSLFHSSALIMIPVYFIVRRKAWSPAIFGLSALFLGMTFLYQKFISVFVVVLENSSYSHYEKWLMTNTNGMNVIKIAVLVLPLFLAFCYKERLRSLWPQIDIVVNLCLLGFLFGLLATKDVIFARFNIYFGLYQMILVPYFVRIFDEKSNALIYIAIVVCYFLYSYLLMPVDSSVLPYRTIFSR

SEQ ID NO: 95 - protein, 344 aa, Bacillus subtilis
  MISC_FEATURE (1)..(344): epsH
METPAVSLLVAVYNTETYIRTCLESLRNQTMDNIEIIIVNDGSADASPDIAEEYAKMDNRFKVIHQENQGLGAVRNKGIEAARGEFIAFIDSDDWIEPDYCEQMLRTAGDETDLVICNYAAEFEDTGKTMDSDIAQTYQDQPKEHYIKALFEGKVRGFSWNKLYRRSMIEAHRLSFPLRGELEHVEDQFFSFRAHFFARSVSYVKTPLYHYRIHLSSIVQRYQKKLFESGLALYETNAAFLQENNKLEEYRKELDTFIVLHSSICMLNEWKTSGSRRLFEKLRNVGVICADPVFQESLSKTGTAPFDAKRSCLLLMAKYRMIPFVAMASAVYQRVIEYKMRNRG

SEQ ID NO: 96 - protein, 358 aa, Bacillus subtilis
  MISC_FEATURE (1)..(358): epsI
MSLQSLKINFAEWLLLKVKYPSQYWLGAADQPVKAAAHQKKIILTLLPSHDNLGDHAIAYASKAFLEQEYPDFDIVEVDMKDIYKSAKSLIRSRHPEDMVFIIGGGNMGDLYRYEEWTRRFIIKTFHDYRVVQLPATAHFSDTKKGRKELKRAQKIYNAHPGLLLMARDETTYQFMKQHFQEKTILKQPDMVLYLDRSKAPAEREGVYMCLREDQESVLQEEQRNRVKAALCEEFGEIKSFTTTIGRRVSRDTREHELEALWSKLQSAEAVVTDRLHGMIFCALTGTPCVVIRSFDHKVMEGYQWLKDIPFMKLIEHPEPERVTAAVNELLTKETSRAGFPRDVYFKGLRDKISGEAQ

SEQ ID NO: 97 - protein, 344 aa, Bacillus subtilis
  MISC_FEATURE (1)..(344): epsJ
MIPLVSIIVPMYNVEPFIEECIDSLLRQTLSDIEIILVNDGTPDRSGEIAEDYAKRDARIRVIHQANGGLSSARNTGIKAARGTYIGFVDGDDYVSSAMFQRLTEEAEQNQLDIVGCGFYKQSSDRRTYVPPQLEANRVLTKPEMTEQLKHAHETRFIWYVWRYLYRRELFERANLLFDEDIRFAEDSPFNLSAFREAERVKMLDEGLYIYRENPNSLTEIPYKPAMDEHLQKQYQAKIAFYNHYGLAGACKEDLNVYICRHQLPMLLANACASPNSPKDIKKKIRQILSYDMVRQAVRHTPFQHEKLLRGERLVLALCKWRLTFLIKLFFEQRGTMKGSAKQA

SEQ ID NO: 98 - protein, 505 aa, Bacillus subtilis
  MISC_FEATURE (1)..(505): epsK
MKFTINFSANLTAFLLSVFLSVWMTPFIVKTLGVEAFGFVHLTQNVINYFSVITVALSSVVVRFFSVAAHRGEREKANAYISNYLAASVLISLLLLLPLAGSAFFIDRVMNVPQALLADVRLSILIGSVLFILTFLMAGFGAAPFYANRLYITSSIQAVQMLIRVLSVLLLFACFAPKIWQIQLAALAGAVIASVLSFYFFKKLIPWFSFRMKDLSFRTSKELFQAGAWSSVNQIGVLLFLQIDLLTANLMLGASASGKYAAIIQFPLLLRSLAGTVASLFAPIMTSYYSKGDMEGLMNYANKAVRLNGLLLALPAALLGGLAGPFLTIWLGPSFSTIAPLLFIHAGYLVVSLAFMPLFYIWTAFNQQKTPAIVTLLLGAVNVVLAVTLSGPAHLGLYGITLAGAISLILKNAIFTPLYVSRITGYKKHVFLKGIIGPLSAAVFAWTVCKAIQFIVKIDSWPSLIATGVTVSFCYAVFAFMLVCTKEERQLVLKRFRKTKGAVNL

SEQ ID NO: 99 - protein, 202 aa, Bacillus subtilis
  MISC_FEATURE (1)..(202): epsL
MILKRLFDLTAAIFLLCCTSVIILFTIAVVRLKIGSPVFFKQVRPGLHGKPFTLYKFRTMTDERDSKGNLLPDEVRLTKTGRLIRKLSIDELPQLLNVLKGDLSLVGPRPLLMDYLPLYTEKQARRHEVKPGITGWAQINGRNAISWEKKFELDVWYVDNWSFFLDLKILCLTVRKVLVSEGIQQTNHVTAERFTGSGDVSS

SEQ ID NO: 100 - protein, 216 aa, Bacillus subtilis
  MISC_FEATURE (1)..(216): epsM
MKNVAIVGDGGHGKVIRELINARSDTRLAAVLDDKFKTFEGGKEWYTGPPKAVTELRRLIPDVLFLIAVGNNSVRKQLAERLGLGKDDFITLIHPSAIVSKSAVIGEGTVIMAGAIIQADARIGAHCIINTGAVAEHDNQISDYVHLSPRATLSGAVSVQEGAHVGTGASVIPQIIIGAWSIVGAGSAVIRSIPDRVTAAGAPARIISSIQTSNKG

SEQ ID NO: 101 - protein, 388 aa, Bacillus subtilis
  MISC_FEATURE (1)..(388): epsN
MHKKIYLSPPHMSGREQHYISEAFRSNWIAPLGPLVNSFEEQLAERVGVKAAAAVGSGTAAIHLALRLLEVKEGDSVFCQSFTFVATANPILYEKAVPVFIDSEPDTWNMSPTALERALEEAKRNGTLPKAVIAVNLYGQSAKMDEIVSLCDAYGVPVIEDAAESLGTVYKGKQSGTFGRFGIFSFNGNKIITTSGGGMLVSNDEAAIEKARFLASQAREPAVHYQHSEIGHNYRLSNILAGVGIAQLEVLDERVEKRRTIFTRYKNALGHLDGVRFMPEYAAGVSNRWLTTLTLDNGLSPYDIVQRLAEENIEARPLWKPLHTQPLFDPALFYSHEDTGSVCEDLFKRGICLPSGSNMTEDEQGRVIEVLLHLFHTVEVKKWTASIR

SEQ ID NO: 102 - protein, 322 aa, Bacillus subtilis
  MISC_FEATURE (1)..(322): epsO
MDSKHSMISLKQKLSGLLDVIPKQSEIIYADYPLYGNVGDLFIMKGTEAFFKEHGIRVRKRWNPDNFPIGRKLDPNLIIVCQGGGNFGDLYPYYQGFREKIVQTYPNHKIVILPQSIYFQNKDNLKRTAEIFSKHANLHIMTREKASYATAQAYFTTNHIQLLPDMAHQLFPVIPTQQPSNQKLRFIRTDHEANQALQEHAEAESYDWRTVLSASDRRTIAFLQTLNVLNKKAGNPLPIAYIWEKYSDYIVQKAIRFFSRYESVETSRLHGHILSSLLQKENTVIDNSYGKNANYFHTWMEGVPSTRLIQHASKKENLPAHM

SEQ ID NO: 103 - protein, 253 aa, Bacillus subtilis
  MISC_FEATURE (1)..(253): tapA
MFRLFHNQQKAKTKLKVLLIFQLSVIFSLTAAICLQFSDDTSAAFHDIETFDVSLQTCKDFQHTDKNCHYDKRWDQSDLHISDQTDTKGTVCSPFALFAVLENTGEKLKKSKWKWELHKLENARKPLKDGNVIEKGFVSNQIGDSLYKIETKKKMKPGIYAFKVYKPAGYPANGSTFEWSEPMRLAKCDEKPTVPKKETKSDVKKENETTQKDIPEKTMKEETSQEAVTKEKETQSDQKESGEEDEKSNEADQ

SEQ ID NO: 104 - protein, 190 aa, Bacillus subtilis
  MISC_FEATURE (1)..(190): sipW
MKLISNILYVIIFTLIIVLTLVVISTRSSGGEPAVFGYTLKSVLSGSMEPEFNTGSLILVKEITDVKELQKGDVITFMQDANTAVTHRIVDITKQGDHLLFKTKGDNNAAADSAPVSDENVRAQYTGFQLPYAGYMLHFASQPIGTAVLLIVPGVMLLVYAFVTISSAIREIERKTKALETDTKDSTMST

SEQ ID NO: 105 - protein, 261 aa, Bacillus subtilis
  MISC_FEATURE (1)..(261): tasA
MGMKKKLSLGVASAALGLALVGGGTWAAFNDIKSKDATFASGTLDLSAKENSASVNLSNLKPGDKLTKDFQFENNGSLAIKEVLMALNYGDFKANGGSNTSPEDFLSQFEVTLLTVGKEGGNGYPKNIILDDANLKDLYLMSAKNDAAAAEKIKKQIDPKFLNASGKVNVATIDGKTAPEYDGVPKTPTDFDQVQMEIQFKDDKTKDEKGLMVQNKYQGNSIKLQFSFEATQWNGLTIKKDHTDKDGYVKENEKAHSEDKN

SEQ ID NO: 106 - protein, 38 aa, Bacillus subtilis
  MISC_FEATURE (1)..(38): phrG
MKRFLIGAGVAAVILSGWFIADHQTHSQEMKVAEKMIG

SEQ ID NO: 107 - protein, 365 aa, Bacillus subtilis
  MISC_FEATURE (1)..(365): rapG
MNKIAPAEIASMLNDWYLAIKKHEVEESSRLFEEVKPLLDDMEEDQEVLAYFSLLELRHKVLLHEARGQGFQHEEPSHMNATSDMLKYYFFLFEGMYEAYKNNYDIAIGLYKDAEQYLDNIPDPIEKAEFHLKVGKLYYKLGQNIVSLNHTRQAVKTFREETDYKKKLASALITMSGNFTEMSQFEEAEAYLDEAIRITSELEDHFFEAQLLHNFGLLHAQSGKSEEAVSKLEEALQNDEYARSAYYYHSAYLLIRELFKIKKKEQALSYYQDVKEKLTAEPNRICEAKIDILYAIYAEGGHAETFHLCKQHMDDLLSEKEYDSVRELSILAGERYRELELYKEAAHFFYEALQIEELIKRTEVI

SEQ ID NO: 108 - protein, 89 aa, Bacillus subtilis
  MISC_FEATURE (1)..(89): remA
MTIKLINIGFGNIISANRMISIVSPESAPIKRMIQDARDRGMLIDATYGRRTRAVVVMDSDHIILSAVQPETVAHRLSVKEEIMDEGQG

SEQ ID NO: 109 - protein, 52 aa, Bacillus subtilis
  MISC_FEATURE (1)..(52): slrA
MKTHVKKDLDKGWHMLIQEARSIGLGIHDVRQFLESETASRKKNHKKTVRQD

SEQ ID NO: 110 - DNA, 540 bp, Bacillus licheniformis
  misc_feature (1)..(540): DNA encoding bslA
ttgaaaagaatgtatcgttctaaattgtcaatcttggcggttagtctcgtgatgatggcttcgattttcctgccttcgttccaggccagcgcccaaacaacaaaaactgaatctgtctaccgcccggctgcgagcgcttccttgtattcggtgataacaggcgccagcaaacaggagtggtcgttctcggatattgagttaacatatcgtcctaactcaatcttggcgctgggaacggttgaatttactttgccgtcgggattttccgccacaacgaaagatacggtgaatggaagagcgctgacaacagggcaaattttgaacaacggaaaaacggtaaggctgccgcttacaattgatctgctgggcattgcggagtttaagctggtgctggccaacaaaacgctgcctgccgcaggaaagtatacattccgggcggagaacagagtactggggctcggatcgactttctatgccgaatcttcaatcgaagtgcaaaaacgggcgactccgcctacacagccgtgcaactgcaagtaa

SEQ ID NO: 111 - DNA, 753 bp, Bacillus licheniformis
  misc_feature (1)..(753): DNA encoding epsA
atgaaagaaaatattgattttagagaactgattgcaatcttgcgaaaaagaacggttcttattctcgttttgacaataggtgtaacattgacgaccggaatcattcagttctatgtgctgacacctgtctatcaggcatcgacgcagatcttggtgcatcaagtaggggagaaaaaggggagcgccacatacagcgatattcaaatcaatcttcaatacacacggacattccaagcgcttttgaaaaacccggtgattttggagcaagtcaagagagagcttgatttaccttactctgccggccggttgggtgaaaaaattgcaacgagcagtgaaagcgaatctgagattataaatatttcggtccaagatgaaaatcagaaacgggcggccgatatagcgaacactttaactgcggtgctcaaaaaagagattaagcaaattatgaacaccgatcgggtaaccgtcctgtcaaaagccgaaatagtcgattcgccgacacctgtcagaccgaattacaaaatgaatattttgctggcattcggcgccgcattaatgaccggaatcgctttggcgttctttttggactttatcgatgatacggttgcaagaccgtctcaagtcgaaaaggaagcgggattcatttatttgggaagtattgagcaaatgaagcataaaaaaagtctgtttcgcggggaccccgatatgaatatccgcgtaaaagcaggaaggagtgagccgcttgggtattag

SEQ ID NO: 112 - DNA, 693 bp, Bacillus licheniformis
  misc_feature (1)..(693): DNA encoding epsB
ttgggtattagaaaaaaacgctctcgcaaatatcaatcggcgcttgtcgcattgcatcagccgaacacgccgatcgtcgaacagtatcggacgatcaggacgaacattgagttttcatcatttgagaagccgttcaagtcattgctcattacatcgggcctgccgggagaaggcaaatcattctcggcttcaaacttggcgatcgtattttcgcaacaggaaaaaaaggtccttttgatcgacgcagatttaagaaagccgacgatccataaaatttttgagctcgataaccattcaggtgtcacaaatgtattaatgaaaaaatcgacgctggaaaatgtcgtccagcaaagccaggcggaaaatctccatgtgctgacaagcggtccgattcctccgaatccgtccgagcttttgtcgtcgcaggcgatggaggacctgcttgcggaagcgtacgaccaatacgatttagtcatccttgattcgccgccgcttttgccggtcgcagacgcgcaaatattggcgaatcaggtggatggaagcattcttgtcatcctcagcggaaaaacaaagcttgataacgcgatcaagtctcgggacgcgctgaattcttccaaaagcgaactgctcggcgccgtgctgaacgggcggaaagtgaagaaagcgcgccaatataattacgcaaccatgtaa

SEQ ID NO: 113 - DNA, 1818 bp, Bacillus licheniformis
  misc_feature (1)..(1818): DNA encoding epsC
ttgacatatcggagaaggctttccattattaccgcactcgattcgtacttggttttgctgtccatctttatcggatatcagctgattttgccatcatatgatttatacccttcggaaatgctgctgatgacttcactgatactgcttggcgctcagcatttattcgcccattgcttccacctttataaaaaagtatgggagtatgcaagcatcggtgaattgtatgtgctgcttaaatcgattacattgtcccatcttgtgacggcggccctcgagctgtttttctttcaaaacgttccggttcgtcttttatgtttaagctggctgttccagctcattttgatcggcggatcgcggatgatgtggcgcatcatcagggaacaggtgaacaaagaaagcaaaggatctctaagggcgcttatcatcggagcgggctctgccggcagtttgattgcaaaacagcttgtgcagaagccggaattgaacattaagcccgtcgcctttatcgacgatgacaaaacgaaataccggcttgaaatcatgggtctgcccgtcttaggcgggaaagagcagattatgcaggcggtccggcaatggaatatcgaccggatcatcatcgccattccatctttgagcgtcactcagatgcaggaaatgtacaaggcgtgtgcgcaaacaggtgtcaaaacgcaaatcatgccgaaaatagacgaaattttgcttggcagacatcctgtcggccagcttcgcgatgtcaaagcagaagatttgcttggaagggagcctgtccagctcgatacgagcgaaatctccaatacggtcaaggaccgcgtcgtacttgtgactggggccggaggttcaatcggctcggaaatctgccggcaaatcagcaaatttaaaccgaaatcgatcattttagtcgggcacggagaaaacagcatccattcgatcctgcttgaactgaaggagaaattcggaaagcatgtcgcctattatcccgaaatcgcggacatacaggacagagaaaaaatgtttttgctgatggaacgctacaaaccgaatgtcatttatcatgcagctgcccacaagcatgtaccgctgatggaaaaatgcccgaaagaagctgtcaaaaacaatatcctcggcacgaaaaacgtcgctgaggccgccgacgaaaccgaagtggagacatttgtcctgatatcgtcagacaaagcggtcaaccctgccaatatcatgggggcaacgaagcggttcgcggaaatgctgatcatgaatctcggcaaaacgagcaaaaccaaatttgtcgccgtccgcttcggaaatgttctcggcagcaggggaagcgtcattccgattttcaaaaaacaaatcgctaaaggcggcccggtcactgtcacacaccaggacatgacgaggtacttcatgacgattccggaagcttcaaggctcgttattcaagcgggggcgcttgccaaaggaagacagatattcgtgctcgatatgggcgaaccggtcaaaatcgtcgacctcgccaaaaatcttatccagctttccggctatacgacagaacagatcaaaatcgaatttacaggcatccggccgggagaaaaaatgtacgaagagctgctgaatcaaaatgaagtgctggcagagcaggtttttccgaagattcatatcggcaaggcggtcgacgtcgaatggacggtgctgaagtcatttatggatgaatttatgtatttgtcagaccgcgagctgagagaacgcttgttcaaagcgatcggccagcacgagaaaaagctggtgacagcgcactag

SEQ ID NO: 114 - DNA, 1149 bp, Bacillus licheniformis
  misc_feature (1)..(1149): DNA encoding epsD
atgacaagaacggttttgttttgcgctactgtggattaccatttcaaggccttccacctcccgtatttaaaatggtttaaagagcaggggtggaatgttcatatcgccgcaaaaggagatatgacactgccctatacagataaaaaatttgatatcgatatcaggcgttctcctttgaatgcaagcaatatcgctgcctatcgggagctggcgcgaatcattgacgaacaccggtacagcatcattcattgccacacgccgatgggaggcgtgctggcaaggcttgcggcccggaaacagagaaaagaggggacgaaagtgatctataccgctcacggtttccacttttgccaaggtgctcctttaaaaaattggctgctgtattatccgattgaaaaagggctgtccgctttgaccgattgcctgattacgatcaatgaagaagatttcgtccttgcaaaaggcttgcgaaaagcgctgcgcacggaaaaaatccacgggatcggcgtcgatacggagcggtttcatcctgtcagtgaaacagagaaaatgctgctcaggaaaacatacggtttcaaagaagacgactttatcctcatatatcccgcggagctgaacgcgaataaaaaccaggccttgctcatcgaaacggcggctgctttaaaagacagagccccgaacttaaaggtcgtgtttgcaggaaaagggcagatggagcaaaaataccgaaatcacgctgaacaaaaaggcgtttcttcgctcgtcatgtttgccggttttcaaaaaaacatccacgaatggattcagcttgcagacgtgtctgtcgcctcaagcatcagggaagggctcggcatgaacctccttgaaggaatggcgtcaggaaagcccgccgttgcagcggacaaccgcggacaccgggaagtcattcaagagggcgtgaacggatttttggttccgcagggagacgccggaacgttcagcgaccggatattgcagctgtaccgtctgccttctttgcgaaaaaagatgggagacgcggggagacgaacagccgccgctttttcccagcagcgcaccgtcaaagaaatggcgggcatttactcttcctttatggataacgaaacagttgaaaggaggctgaaaggatga

SEQ ID NO: 115 - DNA, 843 bp, Bacillus licheniformis
  misc_feature (1)..(843): DNA encoding epsE
atgatagggggtcaaaagccgaaagtttctgtcattatgggagtctacaattgtgagaacacgatcgcagagagcatcgagtcaattttaaatcaaacctataaaaattgggaactgattatatgcgacgatgcttcgacagacgggacatatgctgttgccaggcggtatgccgatcattacgcagataagattaagctgatcaaaaacgagaagaatcagcggctggctgcctcgttaaaccactgtctccaatacgccggcgggaaatatatcgcgcgccaggacggagatgacatctctttgccgaggcggtttgaaaagcaggtcgcgtttttggaatcgcaatctcattatcatgtcgtcggaagcggcatgatggccttcgacgaaaacgggattagaggcgtcagaatgcttccttcctctccagaaccgagaatcatggcgaaagggacgccgttttgccatgcgacgatcatgatgagagccgacgtctacgaggcgctggacggctatcgggtcggccggagaacaagaaggatggaagatgtcgatttgtggcttcgtttttttgaggcgggcttcacgggctacaaccttcaggaagccttatacaaagtaagggaagacgaatcggcgtttaaaaggagaaagctcagctactcgattgataatgcgtttatcgtctttgccgcctgcagacggctgaagctgccgctatcggactatatttatacaatgaaacccatcatcagggggcttatgcctccttttatcatgaacagatatcataaaagaagattgatgaatgaaggcggaggggtcgtaaaacatgaatga

SEQ ID NO: 116 - DNA, 1161 bp, Bacillus licheniformis
  misc_feature (1)..(1161): DNA encoding epsF
atgaatgacggaagcgtgagaccaaaacgggtgcttcacatcgtaagcggaatgaaccgcggcggcgcggagacgatgattatgaatatataccgccacacagacaggcggcatattcaatttgattttatttcccaccgggaagaaacgtgcgattacgacccggagatcatcacgcgcggcggccgggtgttttatgtaccgagcatcggtcggtcgggtcctgtcgcctacatcaaaaacatcagaaggattttggttgagaaagggccttatgccgccgtacacgctcatacggattttcagacgggctttgccgcattggcggccaggctcgccggcgttccggtcagggtctgccattcccacaacacggcctggaagcctaacccccggttttgggatacatggcagcttcttgcattccgccgcttgattttctccagtgctacggctctgtgcgcttgcggcaaagatgccgggcgttttttattcggcgcaaagaagatgggtgaaaacgcggtccatcttttgcaaaacgggattgaacttgaccggttcaaagaagcgaacggcgtttcaaaaacgaatgcgaaaaagagcttcggtatcaaagaagacgcactggtgatcgggcatgtcggccgtttttttgaacagaaaaaccacgcgtttctgctcgggcttgccgcttattgcaagaaatcgggcataccttttcaagcggtgttcgcaggtgacggtccgctgcgcagacagatggaagaaaaagccgctgctctcggtgtaaaagacgacattctgtttctcggcgtcgtcgaagatatcccggctctcatgcaggcatttgatgtatttgtcatgccgtctttgtttgaagggctacccctcgtactggtcgaagcgcaagcgtcagggctcccctgcattgtatcagacaacattacggaagaaaccgatttgggactcggcctgctgcaacgtctcagcttaaatgccggttttgaacggtgggctgaggatatcagccgtgctgctcagccgaaaaagcctgcatggccggaaatagagagaagccttgctgagagaggctatgatgcaaaagcaaatttggcgagactgatggacatctattcaatctccgcagcagaaggacagtga

SEQ ID NO: 117 - DNA, 1104 bp, Bacillus licheniformis
  misc_feature (1)..(1104): DNA encoding epsG
atggctgtctacatgttgaatatggggattgttttcgtctggtcatggtttgcgaaaatgtacggcagggaagatcacaggctgccgacgggctaccgtccgaatgcgatcctcaccgtcgttccgctcgcgtctttgattatcgttgccggcttaagatacaaggtcggaacggattaccacacatatatgctgctttacgaattagccggaaaatacaacagcatttgggagatattcggtttcggaacaggcaagtcgtcgacggacccgggatttaccgcactcttatggattttaaatcagatttcagccgatccggcgctcatgtttgccgtcgttgccgcgattacctatatctatattgtcaagacgctttatgtgtatggaaggccgtttgaattgagcatgtttctgtttatcggcatgtttcactattatgcttcgtttaacggcattcgccaatacatggcggcggccattctgttttgggcggtgcggtatctgatcgatgggaagctggtgcgctatatgatcgttgtactgatctgttcgctttttcattcttcggcattgatcatgattccggtttatttcatcgtcagaagaaaagcgtggtcgcctgtcctctggtgcctgatgctcgtctttttggcggggacttttctgtatcaaaaatttctgtccgtattcctagtcgtgcttgaaaacagtcaatacggacattatgaagaatggctgatgaagaatacaaacggcatgaacgttattaaaatcatcgttcttcttctcccgctcgcacttgccttttgcttcagggagcagctcagaaagcgctggcccgaagtcgattatatcgtaaacctttgtctgatcggtttcctgttcggaattttggcgacaaaggatgtcatttttgcgaggttcaacatttatttcgggctctatcagctgattctcgttccttattttgtacggatttttgaaccgaaatcaaacgcgcttctttatgttttgattttgatctgttacttcttatacagctttatgctcatgccgttcgactcgtcggtattgccgtacagaacgatttttgaacgttga

SEQ ID NO: 118 - DNA, 1032 bp, Bacillus licheniformis
  misc_feature (1)..(1032): DNA encoding epsH
ttggaaaatccagcagttagtctactggtcgctgtttataatacagaagcttttttgccgaattgtttgcagtcgctgatcagccagaccttgaaaaatattgaaatcattatcgtcaatgacggttcaacagacggaagccagaagatcattgatcattacgcccgaaaggacgggcgcattaaaacgattcagcaggacaatcagggcctcggcgctgtccgcaataaaggcatcgaagcggcgtcaggcgagtacttggcattcatcgactccgacgactggattgagccggactattgccagtctatgtatgaaaaagcgaaggatgaggatgccgatcttgtcatctgtgattatgccgttgaaattcaggataccgaaaaaacggtctgcccggacatcgggaagaactatgaaggaaagccgaaagaggcatttatgaaggatttgttaaagggtaaagtaagcggcttttcctggaacaagctctacaggcggagtctgattgagcggcataagctcgttttcccgctccgcgacgagctggaaaacatcgaagatcaatatttcagcttcagatgccttctatatgcgaataccgcggcgttcgtgactaagccgctttatcattacagggtccacctggcgtccatcgtccaaaagtatcaggcagggctgtttgaagacggtctcgctctttatgaagcaaacctggactgcctgacaaagcacggagagctcccggctttaaaggaggcgctgcacgtctttatcgtcaaccacggctgcatcagtattttaaatgaatgcaagagccgaaacaaaaacccttcaatagaaaaatataaaaatatccgcagcatatgcgcctgtccggaattcagggggaaaatctccgcagtggacatgtcggcattcgattccaagaagaagcttttgcttatgctgattcgcttgcggttgatgccggcagtgtatggatttgcggccatttaccagaaaatgatcgagcacagaatgaagaaatag

SEQ ID NO: 119 - DNA, 1080 bp, Bacillus licheniformis
  misc_feature (1)..(1080): DNA encoding epsI
atgacctttcaggaactgaagatcaatcttgcagaatggcttttgctcaaggtgaaatatccttctgaatatgtgatgggaacgccgggtttaaggcgctttgaacaatataaaggcaagaaaaaaatcattctgactttaattccgtcccatgataatttgggagatcatgcgattgccctggcaagccgcacattcatagaaaacgagtttcccgattttgagttaattgaaatcggcattaacgatatttacaaacatgcaaaagcgctcatgcgcatccgccaccccgaggatatggtattcatcatcggcggaggaaacatgggggatttgtaccgcaatgaggaatggacgaggcgcttcattatcaaaacattcaagcattataaaatcgtccagctcccggcaactgcgcacttttccgagacgctccgcggcaaaaaagagctgaaaagggcgaaaaaaatctacaactcccatcgccggctgttcatgatggcgcgcgatgatacaacctatcaatttatgaaacagcacttctcaaaccaaacgattgtaaagcagcctgatatggtgctttatttaaagaaagaacagcagtctgaaagagaaggtgtgttggtttgtttaagggaggataaagaaagctttctcaggccggaagagcgtaaaaagctgctgaaggctgtcggcgacgagtatggcggtgcaaaaacttttacaacgacgatcggcaggagggtcagccgcgtgtcccgcgagaaggagctgaaccggctttgggatcagctgagaggcgcggaggtcgtcgtgacagacaggctgcacggcatgattttttgtgcgataacaggaacgccgtgcgtcgtgatccgctcttttgatcataaggtgcttgaaggctttcgctggctgaaagacgttccttcgatgaagcttgtggaaaatcccgatgcggcagaggttctcggcgcaatcgaagagctggtcaagaccggtgactcgcatcgtgagaccccggcaagggatcattatttcgcagatttaagacggaaaattatgggtgatgtccaatga

SEQ ID NO: 120 - DNA, 1020 bp, Bacillus licheniformis
  misc_feature (1)..(1020): DNA encoding epsJ
atgaagccatttatcagtatcattgttccgatgtacaatgtcgaagattatatagaagaatgcgtcgattcgctgcgcaggcagacgctgaaaaatattgaaatcatccttgttgatgacggctctcccgacagatccggggagatagccagaacgtactgcagcctggatgccagagtgaaagtgattcataaaaaaaacggcgggctgagctcggcgagaaatgccggtcttcaagccgcgacaggagattatgtcgggtttgtcgacggtgatgattttgtattgcctgccatgtttgaaaacatgtacgccgctgccaaaaaagacgacctcgatatcgtcatgtgcgggtatcataaacattccgatacggaagacgcctacttcccgccgccgctgccgaccgatcgcctcttgttgagctgggatattaaacgcgagctcaaaaaggcgcacgaaacccgcttcatctggtatgtgtggcggaatttgtacaggcgcgacctgctgaagaaaaaccagctgtacttttttgaagacattcgttttgcagaggattcgccgtttaatttgtacgccttttatgcagcgaaacgagtgagagcgatcgatgaaggctactacatgtacaggtgcaacccggacagcctgacagaggcgccgtttaaaccgtacatggatgaaagcctgaaaaggcagtaccgggcgaaaaggagattctatgagacgtttcagcttttggatgaatgtgcggacgatttggaaacgtacacatgcaaacatcaaattccgatgcttttagccaatgcctgtgcggaaccgaagccttcaaagcaggtgaggcggcacattaaagacattttgtcttaccggatggtgcagtcgtgcgtcaaagccacgtctctccgcaaccgcaacctgttaatcggccagcggctcgttttattgctatgcaagctgaatattccgattctgctcgaattgttttttaaacgaaatctgcccagtaagggatga

SEQ ID NO: 121 - DNA, 1548 bp, Bacillus licheniformis
  misc_feature (1)..(1548): DNA encoding epsK
ttgaataagacatttgttttgaatctcggcgccaatatggcgtcttttttattatcggtgctgttttcgatgtggctgacgccttacgtcatcaagaccctcggtgttgaggcgtttggatttgtccatcttacacagaatatgatcaattacttttcgataattacggtcgccctgagcgcggtcgttgtccggtttttttctgtttccgcccacaggggagcgcttgatgaagcgagaggctatatgaatacctatatcgtttcatcactggtgctttcagtcatcttgttttttccgctcggcggcacggtgttttttatcgatcagatcattcgcgttcccgccggtcttttgggggatgtgcaggtcgcgcttttgatcggcagcctgctgtttttattgacctttgtgatgtcggggtttgccgccggtccgtttttcgccaataaaatctatattacgagcacgatccaagccatccaaatgctgattcgcgttctgagcgtattgttgattttcgcgtggtttgcaccgaagatctggcacattcagctcgccgctctcatcgcaaccgcttcggcgtgcatcctgtcgatctttttctttaagagattgattccatggtttacgttccgggtgagggatatgtcgtttgcaaagtgcaaaaaactgcttcaggcgggaggatggagctccgtcagccaagtcggcatcctgctgtttctgcaaatcgatttaatggtggcgaatgtgatgctgggggtttccgaatccggcatgtacgcggcgatcattcagtttccgctgttgttgcggacgctttcgggaacgctagcggccgtattttcacctacgattacactctattattcaaaaggcgacaaagaagggctcgtccgttatgccaatcaggccgtcaggtttaacggcatcctgctcgctttgccggcggcgcttctgggggggcttgccggcccgtttctgtcgctttggctcggcccttcattcgaacatttgaaatggctcttactgatacatgcgggctatttggttgtttcattaagcccggcgccgctgttttacatctttaccgcctataacaagctgagaacaccggcgctgaccacggttgccttcggcgttgtcaatttgctgcttgccatcgtgctgagcggcccggcgggactcggcctttacggaattgcgctggccggtgcggcagctttaacgctgaaaaacgtcgtcttcactccgatttatgcatcaaagattacgggtgaaagaaaaagggtcttttataaaggcatatacgggccggttgccggcgcttcatttactttggctgtctgttacgctcttcaatacttattttcgatcgtcagcctgctgtctttgtttgtcacggcactggctgcgacgttggcttacggcctgtttgcctatttcgtcatgttgacgaaagcggaacggcgcattgtcacaacgaagctgcaagcataccggtgttctctatccttcccgtttcaaaagggattttttaaataa

SEQ ID NO: 122 - DNA, 603 bp, Bacillus licheniformis
  misc_feature (1)..(603): DNA encoding epsL
gtgctagcgaaacggtttttcgatcttgcattgtcagttattttgctggtcgcgctcagtccggccatgattctgacggcttgtctcatcagatggaaaatcggctcgcctgttttatttcgccaaacccgtccggggctgaatggcgagccttttacattatataaattcagaacgatgactgatgaaagagatgcagcaggcaatctgctaagcgatgaaaagcggctgacaaagacggggcggctgatcagaaaaacgagccttgacgaattgccgcagctgatcaacgttatcaaaggagacctcagcctcgtcgggccgcgcccgcttttgatggagtatatccccctttatacgaagcggcagtggagacgccacgaagtcaagccgggcatcacgggctgggcgcagatcaacgggagaaataaagtgacatgggaggaaaaatttgaactcgatgtctggtatgtggatcatcgttcttttttgcttgatctcaaaatccttttgttgactgtcgtaaaggttttgaagtcggaaggcgtcagccaggaccggcatgtgacagcagaaaaatttacagggagaaggaatgcctga

SEQ ID NO: 123 - DNA, 627 bp, Bacillus licheniformis
  misc_feature (1)..(627): DNA encoding epsM
atgcaaaacgtggtgatcatcggagccggcggccatgggaaagtcgttcgggaacttgtaaaagagcggccggatacggagcttgccggtattcttgatgaccggtatgcggagcttcatgttgagaacggtctgtatcgagggccttcagctgccgcagaagaacttgcgcggcttcatccggacgcgaagttcgtcctggctgtcggccaaaacagcatcagacagcagctgtatgaacgcattggtcttccgcttgaccggtatgcggttttgattcatccctctgctgttgtcagcggttcggcccggattcaaaacggcgccgttgttatggcatcgagcgtcatccaagcggatgcagacgtcggcatccacgcgattgtcaacacaggtgcgatcgtcgaacacgacaatcggatcggcgattacgttcatctttcgcccggaacggtgttaaccggcggcgtgacagttatggaaggcgctcatctcggcgcgggaacggcggtcattcccggaaagacagtcggacgctggagcgtgacgggagcgggggcagccgtgattcacgacattcctgataattgcaccgcagtcggagtccctgcaagaatgatcaaataa

SEQ ID NO: 124 - DNA, 1146 bp, Bacillus licheniformis
  misc_feature (1)..(1146): DNA encoding epsN
atgagtcagaataagcgaatttatttatcaccgccgcacatgagcggagacgaggagcgctatgtagccgaagcgtttcggacaaactggatcgcgcccctcggtccccttgtcgacacatttgaagaaaagcttgccgcctatgcggggacgtccggagccgcggcagtcagctcaggaacagctgcgatccacctggccttgaaattgctcggcgtcggcaaaggcgatacggtcttctgctcttcttttacgtttgtagcgagcgccaatccgatcatatatgagcaggctgaaccggttttcattgattctgaacgggatacatggaacatgtcgcccgaggcgcttgaacgggcgcttgacgaagcggagcgggccaggaatctcccgaaagccgtcatcgtcgtcaacttgtacggccaaagcgcgaaaatggacgagattatggccatttgcgatcgatttgccgtgcctgtcattgaagatgcagccgaatcgctcggttctgtttataaaggcagaaaaagcgggaccttcggacgcttcggcatttattcgttcaacggtaacaaaatcatcaccacatcgggcggaggaatgctggtcagcgatgatgaagacgcgttgaagaaggcgcgctttttagccactcaggcgcgcgagccagccattcattatcagcacgaaaaagcgggctacaattaccggatgagcaatgttctggccggaatcggcatcgcacagctcgccgttctggatgaccgggtacatgccagacgggcggttttcgagcgctataaggaggcgctttccggtatcgaaggtatagaattcatgcctgaggccggcatgtcaaaccgctggctcacgacattaacgttagacacagcaaagattcaaacaacaccggcggacatcatcgaacagctcgcaaatgaaaacattgaggcccgcccgttatggaagcctttgcacagacagcccctttttaaaggcgcggccttttatccgcacgatgaccagggctctgtctgctgcgacttatttcagcgcgggctctgcctgccgtcaggatcaagtatgacgcgaaaagagcaggaccgggtaattcaaatcgttgccgaccggattaaatataaatga

SEQ ID NO: 125 - DNA, 978 bp, Bacillus licheniformis
  misc_feature (1)..(978): DNA encoding epsO
atggcgattacatattccatggacagcttaaagcataagctggcagaaattttggatgtcattccaaggcattcatcagtcgtttacttggactacccgctatacggaaacgtcggggatctattgatcatgaaaggaacggaagctttttttgaagcatacggcatcaaggtgcgcgaaagatggaatgcggagaatttcattccgggccgccgcattccaaaggacgccatcattgtttgtcaggggggcggcaatttcggcgacttgtaccctcacttccagcagttcagagaacgggtggtcgaacattacccggacaaccggatcgtcattctgccgcagtcgatttattatgagcatgaagaaaatataataaaaacgcgcggcattttggcggctcacccggatctgcacttattcacgcgggaaaaggcatcattcgattttgccgtcaagcgtttcgaagaggtgaaaaacatcaaaatgatgcccgatatggctcaccagctctggccgatcgcggcacctgccgaaaagccgtccgagtccgttctgcggttgatcaggaccgacaaagaagccaacagcagcctgcagaaagcaggggagccggacacgtacgattggaacgtcatcttatcagaaggcgacaaacgcggaatcaaacgcctgcagacgatcaatgtcctcaacaaaaaagcgggcaatccgctgccgatcgcttcttattggaaacgcttttcggacagcctcgtcgacaaatcaatccgtttcttcagccgctatgaatcagtcgttacttcaaggctgcacggccacatcttatcgtgcctgctcggaaaagaaaacgtagtgattgacaactcctacggaaaaaacgcgaattactacaatacatggatgaaagacatcccgaatacgaaactgattcaaaaccatcagacagaagcagaaaaaccgcctgttcacgtatga

SEQ ID NO: 126 - DNA, 729 bp, Bacillus licheniformis
  misc_feature (1)..(729): DNA encoding tapA
atgttccgattatctcgaactaggagatgccgaaaaagagccggaaaaaacattatcgtttttcaaacgctgctgatcatatacctgttaattggattggggagtcagctgtcacagcatacaaacgcatcatttcatgatgtggaaacatacagcatgacaatgaaagcggcctcaacttttcctcaagatgagaagcagtgggacgggagcgatttgaagcttcaaaagcagacgaagacgaaaggaacggtctgtgcaccgctgacattgtttgcagaatataaaaaccgcggcagtcaaatcgccgactctgaatggaagtgggagcttcataaactcggtcattccaaaaagccgctagaggacgggactgtgatcgataaaggcgtgttcaaaaaagaagccgaaagcagcatctaccggatagagtcaaagcgggcagcaaaaggagggctatacgcttttaaattgatcagacctgaaggccatccagatgttaaaaagggcaaacctttcatttggtcaaatgtaatggagcttcaagactgcaatgaagaaacaccgaagcctcctaaaaaagcttcagcaaaaccaaagcaaactgaacagactcaaacaacgcaaaagtctgaacaggatatagaggaaacaccaaaggacagtgaatccgtagataaagaaaatagaaaagaagataattcattagaaagcggggaagcgtcatga

SEQ ID NO: 127 - DNA, 585 bp, Bacillus licheniformis
  misc_feature (1)..(585): DNA encoding sipW
atgaaacatgtgatgaaatggatcagcaatttcttatatgtgattattttcacaatcatcatcgccgcggtcatcgtcgtgatttcgacaaagtcgtccggcggggagccgcagctcttcggctatcaattgaaaacggttttatccggatcgatggagccggagtttaaaacaggttctgtgattgccgttcaaaaagttgaaaatcccgggtctttgaaaaagggagatatcattacattcatgcaagacgaaaacaccatggttacccaccgaattatcggtataacaaaaaataaatcaaatctcatgttcaagacaaagggtgacaataaccaaaaccctgattccgatccggtactggcggaaaatgttgtcgctaagtattcgggcattacggttccgtatgccgggtatttgctggactttgcaagtaaaccgatcggcacagccattttgctgatcgtgccgggactcttgttgattctttatgcagtaattactgtatccgcggctttaagagagattgaccaaaaagctaaagcaattgaagccgctggaaaagatcaatcagtttccatgtaa

SEQ ID NO: 128 - DNA, 795 bp, Bacillus licheniformis
  misc_feature (1)..(795): DNA encoding tasA
atgggtacaaagaaaaaactaggattaggcgttgcgtctgctgcgcttggactggcattagtaggaggaggaacttgggctgcgtttaacgacatcgaaacaactcaagcaacttatgcagcaggtacgcttgacttaaatgcgaaagatacatctgcaagagtgaacttgtccaacttaaaaccaggcgacaaattcactaaagatttcgagttcaaaaatgacggatcacttgcgattaaagaagtgctgatgcaggttggctacagcaatttcgttgacggaaacgcgaaaaacggcggaaaaagcacagcggaagacttcctgaaacaatttaaagtcagcgttctgactgtcggagttgaaggcggtaacggctatcctaaaaacatcattttagatgaagccaacctttatgatctgtacaatatgtccgcgaaaaaagataagaacgcatatgaaaaagtgaagaaagccattgagccagagttcctgcatgacaatggcaaaatcaatgtagcgacaatcaacggaaaaactgcgcctgaatacgacggcattccgaaagacccatatgactttgataaagttcaattggtgattgagtttgtaaatgacaaaacaacagacgccagcggcagaatggtacagaacaaatatcaaggtgattctgtacagcttgacttctcattcgaagctactcaatggaacggcttgacaatcgacggcaaaaaacatgccgatgaaaaaggttatgtgaaagagaacgaaagagcgcacagtgaagataaataa

SEQ ID NO: 129 - DNA, 117 bp, Bacillus licheniformis
  misc_feature (1)..(117): DNA encoding phrG
atgaaaaaactgttcattgttgctgcgattgctgccgtcgtatgttcgggatggtttgcggcagaaactcactgggcatccggcgacatgcaggttgctgaaaagatggtcggttaa

SEQ ID NO: 130 - DNA, 1101 bp, Bacillus licheniformis
  misc_feature (1)..(1101): DNA encoding rapG
atgaacaagatcgccgcggaagaagtcgccaacatccttaatacatggtaccgcgccatcagaagaaatgatgctgaacagtcgatccgaatatttgaagaagtcaaaccgatgctggcagagatggaggaagaccaagaggttttaatctactattctctgctggaactgcggcataaaatcatgctgtatgatacgcggggaaaaaagatagaacagcaagaggagttaacgaacggcggcagtgctgcatcacatatgacatcctattactactacctgttttcaggagcttatgaagtgtataaaaagaattatgagcaggcgatcagcttctataaaattgccgagaagaagcttgctcatgtacatgatgaaattgaggtggcgcaatttcacgataaagtcggaaagctctactattacttgggccagaatatcgtctctttaaaccatacccggcaggcgatggaaattttcaaggggcatggcgaccatgatatgaaccttgtttccacttatattacgatggccggaaattatacagagatggggaaatatacagaggcggaagaatatttaacagaagccatccatacggtaagaaaagccggcgactgttttaaagaaatgcagctccttcataattttgccttgctttatgcggcgatggacaattcggaaaaaagcattcagtttttagaaatcgttttggatgatcaagcatatgctgcatcagattattatttcaatgctgtgtttttaatgatcaaagagctgtttaaagtcggagaccataaacgcgctgcagccttttacaaagaagggaaggaaaggtcgaaatccgcggcgaataaaatatttgacgccaaaatcgatattttatatgcggcttatgcaggagatggtgaacaggcggttaaagactgcaaagacaacattgaaatcctgtttcaaacaaagcaatacgacagcgccagagaactttcgctcttaacggccaatgtttacagatcaaagtcactttataaagaagccgcacatttctttttggaagcgattaaagcggaagaaaaaatgaaaaaagtggagggaatgtga

SEQ ID NO: 131 - DNA, 270 bp, Bacillus licheniformis
  misc_feature (1)..(270): DNA encoding remA
atgacgattaaactgatcaatatcggctttggaaatatcatatccgcgaatcggctgatctcgattgtgagtcctgagtccgcgcctattaagcggatgatccaggatgccagagaccgcggcatgcttatagatgctacatatggaagaagaacccgtgcggttgtcattatggacagtgaccatatcatcttatctgccgtccagcctgagacagtagcacaaaggctttccgttaaagaagaaattatggatgaagggcagggataa

SEQ ID NO: 132 - DNA, 150 bp, Bacillus licheniformis
  misc_feature (1)..(150): DNA encoding slrA
atgaatactcaaacgaaacacaagttggacaaagattgggtgattttgatgaaggaggcgaaaaaacttgggctgcaaattgaagatgtgaaagaatttcttcgcagtcagcccatccaaagcgatgctgaaaaacagataaagcaataa

SEQ ID NO: 133 - protein, 179 aa, Bacillus licheniformis
  MISC_FEATURE (1)..(179): bslA
MKRMYRSKLSILAVSLVMMASIFLPSFQASAQTTKTESVYRPAASASLYSVITGASKQEWSFSDIELTYRPNSILALGTVEFTLPSGFSATTKDTVNGRALTTGQILNNGKTVRLPLTIDLLGIAEFKLVLANKTLPAAGKYTFRAENRVLGLGSTFYAESSIEVQKRATPPTQPCNCK

SEQ ID NO: 134 - protein, 250 aa, Bacillus licheniformis
  MISC_FEATURE (1)..(250): epsA
MKENIDFRELIAILRKRTVLILVLTIGVTLTTGIIQFYVLTPVYQASTQILVHQVGEKKGSATYSDIQINLQYTRTFQALLKNPVILEQVKRELDLPYSAGRLGEKIATSSESESEIINISVQDENQKRAADIANTLTAVLKKEIKQIMNTDRVTVLSKAEIVDSPTPVRPNYKMNILLAFGAALMTGIALAFFLDFIDDTVARPSQVEKEAGFIYLGSIEQMKHKKSLFRGDPDMNIRVKAGRSEPLGY

SEQ ID NO: 135 - protein, 230 aa, Bacillus licheniformis
  MISC_FEATURE (1)..(230): epsB
MGIRKKRSRKYQSALVALHQPNTPIVEQYRTIRTNIEFSSFEKPFKSLLITSGLPGEGKSFSASNLAIVFSQQEKKVLLIDADLRKPTIHKIFELDNHSGVTNVLMKKSTLENVVQQSQAENLHVLTSGPIPPNPSELLSSQAMEDLLAEAYDQYDLVILDSPPLLPVADAQILANQVDGSILVILSGKTKLDNAIKSRDALNSSKSELLGAVLNGRKVKKARQYNYATM

SEQ ID NO: 136 - protein, 605 aa, Bacillus licheniformis
  MISC_FEATURE (1)..(605): epsC
MTYRRRLSIITALDSYLVLLSIFIGYQLILPSYDLYPSEMLLMTSLILLGAQHLFAHCFHLYKKVWEYASIGELYVLLKSITLSHLVTAALELFFFQNVPVRLLCLSWLFQLILIGGSRMMWRIIREQVNKESKGSLRALIIGAGSAGSLIAKQLVQKPELNIKPVAFIDDDKTKYRLEIMGLPVLGGKEQIMQAVRQWNIDRIIIAIPSLSVTQMQEMYKACAQTGVKTQIMPKIDEILLGRHPVGQLRDVKAEDLLGREPVQLDTSEISNTVKDRVVLVTGAGGSIGSEICRQISKFKPKSIILVGHGENSIHSILLELKEKFGKHVAYYPEIADIQDREKMFLLMERYKPNVIYHAAAHKHVPLMEKCPKEAVKNNILGTKNVAEAADETEVETFVLISSDKAVNPANIMGATKRFAEMLIMNLGKTSKTKFVAVRFGNVLGSRGSVIPIFKKQIAKGGPVTVTHQDMTRYFMTIPEASRLVIQAGALAKGRQIFVLDMGEPVKIVDLAKNLIQLSGYTTEQIKIEFTGIRPGEKMYEELLNQNEVLAEQVFPKIHIGKAVDVEWTVLKSFMDEFMYLSDRELRERLFKAIGQHEKKLVTAH

SEQ ID NO: 137 - protein, 382 aa, Bacillus licheniformis
  MISC_FEATURE (1)..(382): epsD
MTRTVLFCATVDYHFKAFHLPYLKWFKEQGWNVHIAAKGDMTLPYTDKKFDIDIRRSPLNASNIAAYRELARIIDEHRYSIIHCHTPMGGVLARLAARKQRKEGTKVIYTAHGFHFCQGAPLKNWLLYYPIEKGLSALTDCLITINEEDFVLAKGLRKALRTEKIHGIGVDTERFHPVSETEKMLLRKTYGFKEDDFILIYPAELNANKNQALLIETAAALKDRAPNLKVVFAGKGQMEQKYRNHAEQKGVSSLVMFAGFQKNIHEWIQLADVSVASSIREGLGMNLLEGMASGKPAVAADNRGHREVIQEGVNGFLVPQGDAGTFSDRILQLYRLPSLRKKMGDAGRRTAAAFSQQRTVKEMAGIYSSFMDNETVERRLKG

SEQ ID NO: 138 - protein, 280 aa, Bacillus licheniformis
  MISC_FEATURE (1)..(280): epsE
MIGGQKPKVSVIMGVYNCENTIAESIESILNQTYKNWELIICDDASTDGTYAVARRYADHYADKIKLIKNEKNQRLAASLNHCLQYAGGKYIARQDGDDISLPRRFEKQVAFLESQSHYHVVGSGMMAFDENGIRGVRMLPSSPEPRIMAKGTPFCHATIMMRADVYEALDGYRVGRRTRRMEDVDLWLRFFEAGFTGYNLQEALYKVREDESAFKRRKLSYSIDNAFIVFAACRRLKLPLSDYIYTMKPIIRGLMPPFIMNRYHKRRLMNEGGGVVKHE

SEQ ID NO: 139 - protein, 386 aa, Bacillus licheniformis
  MISC_FEATURE (1)..(386): epsF
MNDGSVRPKRVLHIVSGMNRGGAETMIMNIYRHTDRRHIQFDFISHREETCDYDPEIITRGGRVFYVPSIGRSGPVAYIKNIRRILVEKGPYAAVHAHTDFQTGFAALAARLAGVPVRVCHSHNTAWKPNPRFWDTWQLLAFRRLIFSSATALCACGKDAGRFLFGAKKMGENAVHLLQNGIELDRFKEANGVSKTNAKKSFGIKEDALVIGHVGRFFEQKNHAFLLGLAAYCKKSGIPFQAVFAGDGPLRRQMEEKAAALGVKDDILFLGVVEDIPALMQAFDVFVMPSLFEGLPLVLVEAQASGLPCIVSDNITEETDLGLGLLQRLSLNAGFERWAEDISRAAQPKKPAWPEIERSLAERGYDAKANLARLMDIYSISAAEGQ

SEQ ID NO: 140 - protein, 367 aa, Bacillus licheniformis
  MISC_FEATURE (1)..(367): epsG
MAVYMLNMGIVFVWSWFAKMYGREDHRLPTGYRPNAILTVVPLASLIIVAGLRYKVGTDYHTYMLLYELAGKYNSIWEIFGFGTGKSSTDPGFTALLWILNQISADPALMFAVVAAITYIYIVKTLYVYGRPFELSMFLFIGMFHYYASFNGIRQYMAAAILFWAVRYLIDGKLVRYMIVVLICSLFHSSALIMIPVYFIVRRKAWSPVLWCLMLVFLAGTFLYQKFLSVFLVVLENSQYGHYEEWLMKNTNGMNVIKIIVLLLPLALAFCFREQLRKRWPEVDYIVNLCLIGFLFGILATKDVIFARFNIYFGLYQLILVPYFVRIFEPKSNALLYVLILICYFLYSFMLMPFDSSVLPYRTIFER

SEQ ID NO: 141 - protein, 343 aa, Bacillus licheniformis
  MISC_FEATURE (1)..(343): epsH
MENPAVSLLVAVYNTEAFLPNCLQSLISQTLKNIEIIIVNDGSTDGSQKIIDHYARKDGRIKTIQQDNQGLGAVRNKGIEAASGEYLAFIDSDDWIEPDYCQSMYEKAKDEDADLVICDYAVEIQDTEKTVCPDIGKNYEGKPKEAFMKDLLKGKVSGFSWNKLYRRSLIERHKLVFPLRDELENIEDQYFSFRCLLYANTAAFVTKPLYHYRVHLASIVQKYQAGLFEDGLALYEANLDCLTKHGELPALKEALHVFIVNHGCISILNECKSRNKNPSIEKYKNIRSICACPEFRGKISAVDMSAFDSKKKLLLMLIRLRLMPAVYGFAAIYQKMIEHRMKK

SEQ ID NO: 142 - protein, 359 aa, Bacillus licheniformis
  MISC_FEATURE (1)..(359): epsI
MTFQELKINLAEWLLLKVKYPSEYVMGTPGLRRFEQYKGKKKIILTLIPSHDNLGDHAIALASRTFIENEFPDFELIEIGINDIYKHAKALMRIRHPEDMVFIIGGGNMGDLYRNEEWTRRFIIKTFKHYKIVQLPATAHFSETLRGKKELKRAKKIYNSHRRLFMMARDDTTYQFMKQHFSNQTIVKQPDMVLYLKKEQQSEREGVLVCLREDKESFLRPEERKKLLKAVGDEYGGAKTFTTTIGRRVSRVSREKELNRLWDQLRGAEVVVTDRLHGMIFCAITGTPCVVIRSFDHKVLEGFRWLKDVPSMKLVENPDAAEVLGAIEELVKTGDSHRETPARDHYFADLRRKIMGDVQ

SEQ ID NO: 143 - protein, 339 aa, Bacillus licheniformis
  MISC_FEATURE (1)..(339): epsJ
MKPFISIIVPMYNVEDYIEECVDSLRRQTLKNIEIILVDDGSPDRSGEIARTYCSLDARVKVIHKKNGGLSSARNAGLQAATGDYVGFVDGDDFVLPAMFENMYAAAKKDDLDIVMCGYHKHSDTEDAYFPPPLPTDRLLLSWDIKRELKKAHETRFIWYVWRNLYRRDLLKKNQLYFFEDIRFAEDSPFNLYAFYAAKRVRAIDEGYYMYRCNPDSLTEAPFKPYMDESLKRQYRAKRRFYETFQLLDECADDLETYTCKHQIPMLLANACAEPKPSKQVRRHIKDILSYRMVQSCVKATSLRNRNLLIGQRLVLLLCKLNIPILLELFFKRNLPSKG

SEQ ID NO: 144 - protein, 515 aa, Bacillus licheniformis
  MISC_FEATURE (1)..(515): epsK
MNKTFVLNLGANMASFLLSVLFSMWLTPYVIKTLGVEAFGFVHLTQNMINYFSIITVALSAVVVRFFSVSAHRGALDEARGYMNTYIVSSLVLSVILFFPLGGTVFFIDQIIRVPAGLLGDVQVALLIGSLLFLLTFVMSGFAAGPFFANKIYITSTIQAIQMLIRVLSVLLIFAWFAPKIWHIQLAALIATASACILSIFFFKRLIPWFTFRVRDMSFAKCKKLLQAGGWSSVSQVGILLFLQIDLMVANVMLGVSESGMYAAIIQFPLLLRTLSGTLAAVFSPTITLYYSKGDKEGLVRYANQAVRFNGILLALPAALLGGLAGPFLSLWLGPSFEHLKWLLLIHAGYLVVSLSPAPLFYIFTAYNKLRTPALTTVAFGVVNLLLAIVLSGPAGLGLYGIALAGAAALTLKNVVFTPIYASKITGERKRVFYKGIYGPVAGASFTLAVCYALQYLFSIVSLLSLFVTALAATLAYGLFAYFVMLTKAERRIVTTKLQAYRCSLSFPFQKGFFK

SEQ ID NO: 145 - protein, 200 aa, Bacillus licheniformis
  MISC_FEATURE (1)..(200): epsL
MLAKRFFDLALSVILLVALSPAMILTACLIRWKIGSPVLFRQTRPGLNGEPFTLYKFRTMTDERDAAGNLLSDEKRLTKTGRLIRKTSLDELPQLINVIKGDLSLVGPRPLLMEYIPLYTKRQWRRHEVKPGITGWAQINGRNKVTWEEKFELDVWYVDHRSFLLDLKILLLTVVKVLKSEGVSQDRHVTAEKFTGRRNA

SEQ ID NO: 146 - protein, 208 aa, Bacillus licheniformis
  MISC_FEATURE (1)..(208): epsM
MQNVVIIGAGGHGKVVRELVKERPDTELAGILDDRYAELHVENGLYRGPSAAAEELARLHPDAKFVLAVGQNSIRQQLYERIGLPLDRYAVLIHPSAVVSGSARIQNGAVVMASSVIQADADVGIHAIVNTGAIVEHDNRIGDYVHLSPGTVLTGGVTVMEGAHLGAGTAVIPGKTVGRWSVTGAGAAVIHDIPDNCTAVGVPARMIK

SEQ ID NO: 147 - protein, 381 aa, Bacillus licheniformis
  MISC_FEATURE (1)..(381): epsN
MSQNKRIYLSPPHMSGDEERYVAEAFRTNWIAPLGPLVDTFEEKLAAYAGTSGAAAVSSGTAAIHLALKLLGVGKGDTVFCSSFTFVASANPIIYEQAEPVFIDSERDTWNMSPEALERALDEAERARNLPKAVIVVNLYGQSAKMDEIMAICDRFAVPVIEDAAESLGSVYKGRKSGTFGRFGIYSFNGNKIITTSGGGMLVSDDEDALKKARFLATQAREPAIHYQHEKAGYNYRMSNVLAGIGIAQLAVLDDRVHARRAVFERYKEALSGIEGIEFMPEAGMSNRWLTTLTLDTAKIQTTPADIIEQLANENIEARPLWKPLHRQPLFKGAAFYPHDDQGSVCCDLFQRGLCLPSGSSMTRKEQDRVIQIVADRIKYK

SEQ ID NO: 148 - protein, 325 aa, Bacillus licheniformis
  MISC_FEATURE (1)..(325): epsO
MAITYSMDSLKHKLAEILDVIPRHSSVVYLDYPLYGNVGDLLIMKGTEAFFEAYGIKVRERWNAENFIPGRRIPKDAIIVCQGGGNFGDLYPHFQQFRERVVEHYPDNRIVILPQSIYYEHEENIIKTRGILAAHPDLHLFTREKASFDFAVKRFEEVKNIKMMPDMAHQLWPIAAPAEKPSESVLRLIRTDKEANSSLQKAGEPDTYDWNVILSEGDKRGIKRLQTINVLNKKAGNPLPIASYWKRFSDSLVDKSIRFFSRYESVVTSRLHGHILSCLLGKENVVIDNSYGKNANYYNTWMKDIPNTKLIQNHQTEAEKPPVHV

SEQ ID NO: 149 - protein, 242 aa, Bacillus licheniformis
  MISC_FEATURE (1)..(242): tapA
MFRLSRTRRCRKRAGKNIIVFQTLLIIYLLIGLGSQLSQHTNASFHDVETYSMTMKAASTFPQDEKQWDGSDLKLQKQTKTKGTVCAPLTLFAEYKNRGSQIADSEWKWELHKLGHSKKPLEDGTVIDKGVFKKEAESSIYRIESKRAAKGGLYAFKLIRPEGHPDVKKGKPFIWSNVMELQDCNEETPKPPKKASAKPKQTEQTQTTQKSEQDIEETPKDSESVDKENRKEDNSLESGEAS

SEQ ID NO: 150 - protein, 194 aa, Bacillus licheniformis
  MISC_FEATURE (1)..(194): sipW
MKHVMKWISNFLYVIIFTIIIAAVIVVISTKSSGGEPQLFGYQLKTVLSGSMEPEFKTGSVIAVQKVENPGSLKKGDIITFMQDENTMVTHRIIGITKNKSNLMFKTKGDNNQNPDSDPVLAENVVAKYSGITVPYAGYLLDFASKPIGTAILLIVPGLLLILYAVITVSAALREIDQKAKAIEAAGKDQSVSM

SEQ ID NO: 151 - protein, 264 aa, Bacillus licheniformis
  MISC_FEATURE (1)..(264): tasA
MGTKKKLGLGVASAALGLALVGGGTWAAFNDIETTQATYAAGTLDLNAKDTSARVNLSNLKPGDKFTKDFEFKNDGSLAIKEVLMQVGYSNFVDGNAKNGGKSTAEDFLKQFKVSVLTVGVEGGNGYPKNIILDEANLYDLYNMSAKKDKNAYEKVKKAIEPEFLHDNGKINVATINGKTAPEYDGIPKDPYDFDKVQLVIEFVNDKTTDASGRMVQNKYQGDSVQLDFSFEATQWNGLTIDGKKHADEKGYVKENERAHSEDK

SEQ ID NO: 152 - protein, 38 aa, Bacillus licheniformis
  MISC_FEATURE (1)..(38): phrG
MKKLFIVAAIAAVVCSGWFAAETHWASGDMQVAEKMVG

SEQ ID NO: 153 - protein, 366 aa, Bacillus licheniformis
  MISC_FEATURE (1)..(366): rapG
MNKIAAEEVANILNTWYRAIRRNDAEQSIRIFEEVKPMLAEMEEDQEVLIYYSLLELRHKIMLYDTRGKKIEQQEELTNGGSAASHMTSYYYYLFSGAYEVYKKNYEQAISFYKIAEKKLAHVHDEIEVAQFHDKVGKLYYYLGQNIVSLNHTRQAMEIFKGHGDHDMNLVSTYITMAGNYTEMGKYTEAEEYLTEAIHTVRKAGDCFKEMQLLHNFALLYAAMDNSEKSIQFLEIVLDDQAYAASDYYFNAVFLMIKELFKVGDHKRAAAFYKEGKERSKSAANKIFDAKIDILYAAYAGDGEQAVKDCKDNIEILFQTKQYDSARELSLLTANVYRSKSLYKEAAHFFLEAIKAEEKMKKVEGM

SEQ ID NO: 154 - protein, 89 aa, Bacillus licheniformis
  MISC_FEATURE (1)..(154): remA
MTIKLINIGFGNIISANRLISIVSPESAPIKRMIQDARDRGMLIDATYGRRTRAVVIMDSDHIILSAVQPETVAQRLSVKEEIMDEGQG

SEQ ID NO: 155 - protein, 49 aa, Bacillus licheniformis
  MISC_FEATURE (1)..(49): slrA
MNTQTKHKLDKDWVILMKEAKKLGLQIEDVKEFLRSQPIQSDAEKQIKQ

SEQ ID NO: 156 - DNA, 141 bp, Bacillus subtilis
  misc_feature (1)..(141): DNA encoding degQ
atggaaaagaaacttgaagaagtaaaacaattgttattccgactcgaacttgatattaaagaaacgacagattcattacgaaacattaacaaaagcattgatcaactcgataaatacaattatgcaatgaaaatttcgtga

SEQ ID NO: 157 - DNA, 183 bp, Bacillus subtilis
  misc_feature (1)..(183): DNA encoding degR
atggatgataaagacttgaagttgatccttcacaaaacatttatagaaatatacagtgatttagaagaactggccgatatcgcgaaaaaaggaaaaccatcaatggaaaagtatgttgaagagattgaacagaggtgtaaacaaaacattttggcgattgaaatccagatgaaaatcaaatag

SEQ ID NO: 158 - DNA, 1158 bp, Bacillus subtilis
  misc_feature (1)..(1158): DNA encoding degS
atgaataaaacaaagatggattccaaagtgctggattctattttgatgaagatgctgaaaaccgttgacgggagcaaggacgaggtttttcaaatcggggagcagtcacgccagcagtatgaacagctggtcgaagaactgaaacaaattaaacagcaggtgtatgaagtgattgagcttggcgataaacttgaagtgcaaactcgccatgcgagaaaccgtttatccgaggtcagccgtaattttcatagattcagtgaagaggaaatccgcaatgcttatgaaaaagcccataagctgcaggtagaattgacgatgatccagcagcgtgagaagcaattgcgcgaacggcgggacgatttggagcgcagattgctagggcttcaggaaatcattgagcggtcagaatcattagtaagccaaattacagttgtgctcaactacttgaatcaggatttgcgcgaagttggactgcttcttgctgatgctcaggcaaaacaggatttcggcttaagaattattgaggcgcaggaagaagagcgaaaaagagtctcaagagaaatccatgacggacccgctcaaatgctggcgaatgttatgatgagatcggaattaatcgagcggattttccgtgaccggggcgcagaggacggattccaagaaattaaaaatctccgccaaaatgttcggaatgccctttacgaagtgagaaggattatatatgatttaagaccgatggcccttgatgacctaggcctgattccaactttaagaaaatatctatatacaaccgaggaatataacgggaaggtcaaaatacattttcagtgcattggagaaacagaggatcagaggctagcgcctcagtttgaggttgcgctcttcaggctcgcacaggaagctgtgtctaatgcgctaaagcattctgaatctgaagaaattacagtcaaagttgagatcacaaaggattttgtgattttaatgataaaagataacggtaaagggttcgacctgaaggaagcgaaagagaagaaaaacaaatcattcggcttgctgggcatgaaagaaagagtagatttattggaaggaacgatgacaatagattcgaaaataggtcttgggacatttattatgattaaggttccgttatctctttga

SEQ ID NO: 159 - DNA, 690 bp, Bacillus subtilis
  misc_feature (1)..(690): DNA encoding degU
gtgactaaagtaaacattgttattatcgacgaccatcagttatttcgtgaaggtgttaaacggatattggattttgaacctacctttgaagtggtagccgaaggtgatgacggggacgaagcggctcgtattgttgagcactatcatcctgatgttgtgatcatggatatcaatatgccaaacgtaaatggtgtggaagctacaaaacagcttgtagagctgtatcctgaatctaaagtaattattctatcaattcacgatgacgaaaattatgtaacacatgccctgaaaacaggtgcaagaggttatctgctgaaagagatggatgctgatacattaattgaagcggttaaagtagtggctgagggcggatcttacctccatccgaaggttactcacaacctcgttaacgaattccgccgccttgcaacaagcggagtttctgcacaccctcaacatgaggtttaccctgaaatccgcagaccattacatattttaactaggcgggaatgtgaagtgctgcagatgcttgcagacggaaaaagcaaccgcggtattggtgaatcattgtttatcagtgagaaaaccgttaaaaaccatgtcagcaatattttacaaaaaatgaatgtaaacgaccggacgcaagccgttgtggtcgccattaaaaatggctgggtagaaatgagatag

SEQ ID NO: 160 - protein, 46 aa, Bacillus subtilis
  MISC_FEATURE (1)..(46): DegQ
MEKKLEEVKQLLFRLELDIKETTDSLRNINKSIDQLDKYNYAMKIS

SEQ ID NO: 161 - protein, 60 aa, Bacillus subtilis
  MISC_FEATURE (1)..(60): DegR
MDDKDLKLILHKTFIEIYSDLEELADIAKKGKPSMEKYVEEIEQRCKQNILAIEIQMKIK

SEQ ID NO: 162 - protein, 385 aa, Bacillus subtilis
  MISC_FEATURE (1)..(385): DegS
MNKTKMDSKVLDSILMKMLKTVDGSKDEVFQIGEQSRQQYEQLVEELKQIKQQVYEVIELGDKLEVQTRHARNRLSEVSRNFHRFSEEEIRNAYEKAHKLQVELTMIQQREKQLRERRDDLERRLLGLQEIIERSESLVSQITVVLNYLNQDLREVGLLLADAQAKQDFGLRIIEAQEEERKRVSREIHDGPAQMLANVMMRSELIERIFRDRGAEDGFQEIKNLRQNVRNALYEVRRIIYDLRPMALDDLGLIPTLRKYLYTTEEYNGKVKIHFQCIGETEDQRLAPQFEVALFRLAQEAVSNALKHSESEEITVKVEITKDFVILMIKDNGKGFDLKEAKEKKNKSFGLLGMKERVDLLEGTMTIDSKIGLGTFIMIKVPLSL

SEQ ID NO: 163 - protein, 229 aa, Bacillus subtilis
  MISC_FEATURE (1)..(229): DegU
MTKVNIVIIDDHQLFREGVKRILDFEPTFEVVAEGDDGDEAARIVEHYHPDVVIMDINMPNVNGVEATKQLVELYPESKVIILSIHDDENYVTHALKTGARGYLLKEMDADTLIEAVKVVAEGGSYLHPKVTHNLVNEFRRLATSGVSAHPQHEVYPEIRRPLHILTRRECEVLQMLADGKSNRGIGESLFISEKTVKNHVSNILQKMNVNDRTQAVVVAIKNGWVEMR

SEQ ID NO: 164 - protein, 385 aa, Bacillus subtilis
  MISC_FEATURE (1)..(385): DegS_S76D
MNKTKMDSKVLDSILMKMLKTVDGSKDEVFQIGEQSRQQYEQLVEELKQIKQQVYEVIELGDKLEVQTRHARNRLDEVSRNFHRFSEEEIRNAYEKAHKLQVELTMIQQREKQLRERRDDLERRLLGLQEIIERSESLVSQITVVLNYLNQDLREVGLLLADAQAKQDFGLRIIEAQEEERKRVSREIHDGPAQMLANVMMRSELIERIFRDRGAEDGFQEIKNLRQNVRNALYEVRRIIYDLRPMALDDLGLIPTLRKYLYTTEEYNGKVKIHFQCIGETEDQRLAPQFEVALFRLAQEAVSNALKHSESEEITVKVEITKDFVILMIKDNGKGFDLKEAKEKKNKSFGLLGMKERVDLLEGTMTIDSKIGLGTFIMIKVPLSL

SEQ ID NO: 165 - protein, 229 aa, Bacillus subtilis
  MISC_FEATURE (1)..(229): DegU_H12L
VTKVNIVIIDDLQLFREGVKRILDFEPTFEVVAEGDDGDEAARIVEHYHPDVVIMDINMPNVNGVEATKQLVELYPESKVIILSIHDDENYVTHALKTGARGYLLKEMDADTLIEAVKVVAEGGSYLHPKVTHNLVNEFRRLATSGVSAHPQHEVYPEIRRPLHILTRRECEVLQMLADGKSNRGIGESLFISEKTVKNHVSNILQKMNVNDRTQAVVVAIKNGWVEMR

SEQ ID NO: 166 - DNA, 141 bp, Bacillus licheniformis
  misc_feature (1)..(141): DNA encoding degQ
gtggaaaagcaacaaattgaagaattaaaacaactgctttggcggctagagaatgaaatcagagaaacaaaggactccttgcgcaagattaacaaaagcattgatcaatacgataagtacacatatctaaaaacctcgtaa

SEQ ID NO: 167 - DNA, 183 bp, Bacillus licheniformis
  misc_feature (1)..(183): DNA encoding degR
gtggaaagcaatgaactgcaaaccgttcttcacaagacatttgtggaggtatataaagatcttgaacagctcgttgatatcgctaaaaaggggcgtccatctctcgagaagaacattgaggaaatcgagcaaaggctgaagcaaaatatactggcgatcgaaattcagctgaaaattaaatag

SEQ ID NO: 168 - DNA, 1158 bp, Bacillus licheniformis
  misc_feature (1)..(1158): DNA encoding degS
gtgagcgtttccaaaatggactccaaagttttagattcaatcattatgaagatgttaaaaacggttgacgggagcaaggatgaagtctttcaaatcggagagcagtcccgccagcaatacgaaggcttggtagaagagctgaaacagattaaacagcaggtcaacgaagtcatcgatcttggagacaggctggaagtgcatgcccgccatgcgcggaaccgcttgtcagaggtcagcagaaactttcataaattcagtgaagaagagattcgcgaagcttatgaaaaagcccacaaactgcaggttgaactgacaatgatccagcagcgggaaaagcagctgagagagaagcgtgatgatctggaacggcgccttttggggcttcaggaaatcatcgagcgttcagaagggcttgtcagtcagattaccgtcgtcttaaactacttaaatcaggatctgcgccaggtcggcgttctccttgaagatgcgcaggccaagcaggatttcggactgcggattatcgaagcccaggaggaagaaagaaaaagggtctcaagggaaattcacgacggtcctgcgcaaatgctggccaacgttatgatgagatctgaactgatcgaacgcatcttcagagacaaaggaacagaagaaggctttcaggaaattaaaaacctccggcaaaacgtcagaaatgctctttatgaagtcagaagaatcatttatgatttaaggccgatggctttagacgatttgggattgataccgacgctcagaaaatacttgaacacgatcgaagattatcatggaaaagcaaagattcatttccaatgcatcggagaatccgaagaaagaagaatagcaccgcggtttgaggttgcactattccggcttgcacaggaagcggtgacaaacgccttaaaacactccgaatcaactgaaattcatgttaaagtagaagtgacaaaagattttgtgacgctgattatcaaagacaatggaaacggctttgacttaaaagaagtaaaaggcaagaagaacaaatctttcggtctgctaggtatgaaagaaagagtcgatttgctcgaaggctcaatgacaatcgattcgaaaataggtcttgggacatttatattgattaaagttccactgtctttgtaa

SEQ ID NO: 169 - DNA, 690 bp, Bacillus licheniformis
  misc_feature (1)..(690): DNA encoding degU
gtgactaaagtaaatattgtaattattgacgatcatcagttattccgtgaaggtgtcaaacggattttggatttcgaacctacctttgaggtagtggccgaaggagacgacggagatgaagcggctcgcattgtcgagcactaccatcctgatgttgttatcatggatattaatatgccgaatgtgaacggagtagaagcgacaaaacaactggtcgacttgtatccggaatcaaaggttattattttatccatccatgatgacgaaaactatgttacacatgcattaaaaacaggagcccggggctatctgctgaaagaaatggatgccgatacgctgatcgaagccgtgaaagtagtagctgaaggcggatcttatctgcatcctaaggttacacacaatcttgtgaatgaattccgccgtcttgcaacaagcggtgtatcatctcacgctcagcatgaggtgtatccggaaatccggagacctcttcacattctcacaagaagggaatgcgaggtactgcagatgctggcggatggaaaaagcaaccgcggaatcggcgaatcattatttatcagtgaaaaaacggttaaaaaccatgtcagcaacatccttcaaaaaatgaatgtaaacgacagaacgcaggctgttgttgtagccattaaaaacggctgggtagaaatgagataa

SEQ ID NO: 170 - protein, 46 aa, Bacillus licheniformis
  MISC_FEATURE (1)..(46): DegQ
MEKQQIEELKQLLWRLENEIRETKDSLRKINKSIDQYDKYTYLKTS

SEQ ID NO: 171 - protein, 60 aa, Bacillus licheniformis
  MISC_FEATURE (1)..(60): DegR
MESNELQTVLHKTFVEVYKDLEQLVDIAKKGRPSLEKNIEEIEQRLKQNILAIEIQLKIK

SEQ ID NO: 172 - protein, 385 aa, Bacillus licheniformis
  MISC_FEATURE (1)..(385): DegS
MSVSKMDSKVLDSIIMKMLKTVDGSKDEVFQIGEQSRQQYEGLVEELKQIKQQVNEVIDLGDRLEVHARHARNRLSEVSRNFHKFSEEEIREAYEKAHKLQVELTMIQQREKQLREKRDDLERRLLGLQEIIERSEGLVSQITVVLNYLNQDLRQVGVLLEDAQAKQDFGLRIIEAQEEERKRVSREIHDGPAQMLANVMMRSELIERIFRDKGTEEGFQEIKNLRQNVRNALYEVRRIIYDLRPMALDDLGLIPTLRKYLNTIEDYHGKAKIHFQCIGESEERRIAPRFEVALFRLAQEAVTNALKHSESTEIHVKVEVTKDFVTLIIKDNGNGFDLKEVKGKKNKSFGLLGMKERVDLLEGSMTIDSKIGLGTFILIKVPLSL

SEQ ID NO: 173 - protein, 229 aa, Bacillus licheniformis
  MISC_FEATURE (1)..(229): DegU
MTKVNIVIIDDHQLFREGVKRILDFEPTFEVVAEGDDGDEAARIVEHYHPDVVIMDINMPNVNGVEATKQLVDLYPESKVIILSIHDDENYVTHALKTGARGYLLKEMDADTLIEAVKVVAEGGSYLHPKVTHNLVNEFRRLATSGVSSHAQHEVYPEIRRPLHILTRRECEVLQMLADGKSNRGIGESLFISEKTVKNHVSNILQKMNVNDRTQAVVVAIKNGWVEMR

SEQ ID NO: 174 - protein, 385 aa, Bacillus licheniformis
  MISC_FEATURE (1)..(385): DegS_S76D
MSVSKMDSKVLDSIIMKMLKTVDGSKDEVFQIGEQSRQQYEGLVEELKQIKQQVNEVIDLGDRLEVHARHARNRLDEVSRNFHKFSEEEIREAYEKAHKLQVELTMIQQREKQLREKRDDLERRLLGLQEIIERSEGLVSQITVVLNYLNQDLRQVGVLLEDAQAKQDFGLRIIEAQEEERKRVSREIHDGPAQMLANVMMRSELIERIFRDKGTEEGFQEIKNLRQNVRNALYEVRRIIYDLRPMALDDLGLIPTLRKYLNTIEDYHGKAKIHFQCIGESEERRIAPRFEVALFRLAQEAVTNALKHSESTEIHVKVEVTKDFVTLIIKDNGNGFDLKEVKGKKNKSFGLLGMKERVDLLEGSMTIDSKIGLGTFILIKVPLSL

SEQ ID NO: 175 - protein, 229 aa, Bacillus licheniformis
  MISC_FEATURE (1)..(229): DegU_H12L
MTKVNIVIIDDLQLFREGVKRILDFEPTFEVVAEGDDGDEAARIVEHYHPDVVIMDINMPNVNGVEATKQLVDLYPESKVIILSIHDDENYVTHALKTGARGYLLKEMDADTLIEAVKVVAEGGSYLHPKVTHNLVNEFRRLATSGVSSHAQHEVYPEIRRPLHILTRRECEVLQMLADGKSNRGIGESLFISEKTVKNHVSNILQKMNVNDRTQAVVVAIKNGWVEMR

SEQ ID NO: 176 - protein, 46 aa, Bacillus pumilus
  MISC_FEATURE (1)..(46): DegQ Bacillus pumilus strain SH-B9
MEKYEIEELKQLLWKLENEIRETTASLHNINKSIDQYDKYEYVKIS

SEQ ID NO: 177 - protein, 46 aa, Bacillus velezensis sp.
  MISC_FEATURE (1)..(46): DegQ Bacillus velezensis FZB42
MENKLEEVKQLLFRLENDIRETTDSLRNINKSIDQLDKFSYAMKIS

SEQ ID NO: 178 - protein, 46 aa, Bacillus amyloliquefaciens
  MISC_FEATURE (1)..(46): DegQ Bacillus amyloliquefaciens XH7
MEKKLEEVKQLLFRLENDIRETTDSLRNINKSIDQLDKFSYAMKIS

SEQ ID NO: 179 - DNA, 221 bp, Bacillus subtilis
  misc_feature (1)..(221): Promoter 5-prime region to degQ gene of Bacillus subtilis 168
atataaacgctctttcgcatagaaagatagtcccgttcttgttgtgattcctttgagtataaggacctattgagatttgcggtgtcacgcaggacttttttgcatacttttcggtgaaaaatgagccgaaagcagacacactattagtaacagatcaaatacctaggactcgttcaccatacacaattcattgatctttcaaaaaaaggagtgtggaaacg

SEQ ID NO: 180 - DNA, 221 bp, Bacillus subtilis
  misc_feature (1)..(221): Promoter 5-prime region to degQ gene of Bacillus subtilis strain NCIB 3610
atataaacgctctttcgcatagaaagatagtcccgttcttgttgtgattcctttgagtataaggacctattgagatttgcggtgtcacgcaggacttttttgcatacttttcggtgaaaaatgagccgaaagcagatacactattagtaacagatcaaatacctaggactcgttcaccatacacaattcattgatctttcaaaaaaaggagtgtggaaacg

SEQ ID NO: 181 - DNA, 277 bp, Bacillus licheniformis
  misc_feature (1)..(277): Promoter 5-prime region to degQ gene of Bacillus licheniformis strain ATCC 14580 = DSM 13
acttgcgatgataaaggggtttatatgcgatctttatttgactccttgaaaaacaaacaaaaattttatgtttcattaggatttgagaactaatcgcaagttgtgtaaaatgggtgttatggtctatttaaagtttgcggtgtaacgcatgaatttatatgcaacttttcggtgaaaaagaaaccaaatccctttaaacttgtattaacagatcaaatacctatgactcgttcactatacacaaattgattgatcttccaaaaggagtgtggaaccg

SEQ ID NO: 182 - DNA, 505 bp, Bacillus pumilus
  misc_feature (1)..(505): Promoter 5-prime region to degQ gene of Bacillus pumilus strain SH-B9
atggtcacctcttctctcattgacgtgaaattggacggatatctgttctaaatgtatgtagaaaatcagaataaagaatcaaaatcatctttctatctttataaatccaacataaaaataggtacttttgcggtcaatttcggtgaatgtcgccaaaaaattgcggtgtccacaaaggggcctgtcgtctgacctataacacgcaaaaccgaggttacaggagggatcgtatgttccgttgtgagagaagtgtgacgaatgatgcctattcttgtcgcaaaaatgacggagttcctttgaaattaagataaaagtatgatttgaaaagtcagaaaatgatgaaaaaagacctattgaattttgcggtatcacgcacacattttgtgcatacttttcggtgaaaaaatactcaattacatttacactgttagtaacagatcaaatacttatgacccaatcaccatacacaaatacattgatcttccaaaaaaggagagtggaatcg

SEQ ID NO: 183 - DNA, 464 bp, Bacillus velezensis sp.
  misc_feature (1)..(464): Promoter 5-prime region to degQ gene of Bacillus velezensis FZB42
acggcggcctccttcctgagaaaacattcgttatatatgagtataggcggcgtgataacgagttatgacatgcaaaagaccacaatgcgggggttgcggtcttttcggtgtttgtcggtggttatgcgacgctgttcgcccattctgttttgaaattgcgacggcagggactatagtccctgccggtttgtcggaaaaccgttaaaaaaacagcagaaccgccagattggtctgcttcatcctaaacgtctgcctttctggtagttatatagtcctgttcgctaaatgcttcattcgggactatgggattaccccggtttgcggtgtcacgcagatacttttacacatacttttcggtgaaaaatcccgcaaaaacgtttacactattagtaacagatcaaatacctaggactcgttcaccatacacaattcattgatctttcaaaaaaaggagtgtggaaacg

SEQ ID NO: 184 - DNA, 464 bp, Bacillus amyloliquefaciens
  misc_feature (1)..(464): Promoter 5-prime region to degQ gene of Bacillus amyloliquefaciens XH7
gcggcggcctccttctgagataacattcgttatacatgagtataggcggcgtgataacgagttatgacatgcaaaaagaccacaatgcgggtgttgcggtcttttcggtgtttgtcggtggttatgcgacgctgttcgcccattctcttttgaaattgcgacgtcagggactatagtccttagcggtttgtcggaaaaccgttaaaaaaccagcagaaccaccagattgatctgcttcatcccaaacgtctgcctttatggtagttatatagtcctgttcgccaaatgctctgttcgggactatgggattaccgtggtttgcggtgtcacgcagatacttttacacatacttttcggtgaaaaatcccgcaaaaacgtttacactattagtaacagatcaaatacctaggactcgttcaccatacacaattcattgatctttcaaaaaaaggagtgtggaaacg

SEQ ID NO: 185 - protein, 385 aa, Bacillus subtilis
  MISC_FEATURE (1)..(385): DegS_S218E
MNKTKMDSKVLDSILMKMLKTVDGSKDEVFQIGEQSRQQYEQLVEELKQIKQQVYEVIELGDKLEVQTRHARNRLSEVSRNFHRFSEEEIRNAYEKAHKLQVELTMIQQREKQLRERRDDLERRLLGLQEIIERSESLVSQITVVLNYLNQDLREVGLLLADAQAKQDFGLRIIEAQEEERKRVSREIHDGPAQMLANVMMRSELIERIFRDRGAEDEFQEIKNLRQNVRNALYEVRRIIYDLRPMALDDLGLIPTLRKYLYTTEEYNGKVKIHFQCIGETEDQRLAPQFEVALFRLAQEAVSNALKHSESEEITVKVEITKDFVILMIKDNGKGFDLKEAKEKKNKSFGLLGMKERVDLLEGTMTIDSKIGLGTFIMIKVPLSL

SEQ ID NO: 186 - protein, 385 aa, Bacillus licheniformis
  MISC_FEATURE (1)..(385): DegS_S218E
MSVSKMDSKVLDSIIMKMLKTVDGSKDEVFQIGEQSRQQYEGLVEELKQIKQQVNEVIDLGDRLEVHARHARNRLSEVSRNFHKFSEEEIREAYEKAHKLQVELTMIQQREKQLREKRDDLERRLLGLQEIIERSEGLVSQITVVLNYLNQDLRQVGVLLEDAQAKQDFGLRIIEAQEEERKRVSREIHDGPAQMLANVMMRSELIERIFRDKGTEEEFQEIKNLRQNVRNALYEVRRIIYDLRPMALDDLGLIPTLRKYLNTIEDYHGKAKIHFQCIGESEERRIAPRFEVALFRLAQEAVTNALKHSESTEIHVKVEVTKDFVTLIIKDNGNGFDLKEVKGKKNKSFGLLGMKERVDLLEGSMTIDSKIGLGTFILIKVPLSL

SEQ ID NO: 187 - DNA, 381 bp, Bacillus licheniformis
  misc_feature (1)..(381): B. licheniformis DSM641; 5-prime region to aprE gene comprising promoter aprE
atctttcacccgtttctgtatgcgatatattgcatattttaatagatgatcgacaaggccgcaacctccttcggcaaaaaatgatctcataaaataaatgaatagtattttcataaaatgaatcagatggagcaatctcctgtcattcgcggccctcgggacctctttccctgccaggctgaagcggtctattcatactttcgaactgaacatttttctaaaacagttattaataaccaaaaaattttaaattggtcctccaaaaaaataggcctaccatataattcattttttttctataataaattaacagaataattggaatagattatattatccttctatttaaattattctgaataaagaggaggagagtgagta

SEQ ID NO: 188 - DNA, 227 bp, Bacillus licheniformis
  misc_feature (1)..(227): B. licheniformis DSM641; Promoter aprE trunc. DSM641
ctcgggacctctttccctgccaggctgaagcggtctattcatactttcgaactgaacatttttctaaaacagttattaataaccaaaaaattttaaattggtcctccaaaaaaataggcctaccatataattcattttttttctataataaattaacagaataattggaatagattatattatccttctatttaaattattctgaataaagaggaggagagtgagta

SEQ ID NO: 189 - DNA, 375 bp, Bacillus licheniformis
  misc_feature (1)..(375): B. licheniformis DSM641; Promoter aprE fl- DSM641
cacccgtttctgtatgcgatatattgcatattttaatagatgatcgacaaggccgcaacctccttcggcaaaaaatgatctcataaaataaatgaatagtattttcataaaatgaatcagatggagcaatctcctgtcattcgcggccctcgggacctctttccctgccaggctgaagcggtctattcatactttcgaactgaacatttttctaaaacagttattaataaccaaaaaattttaaattggtcctccaaaaaaataggcctaccatataattcattttttttctataataaattaacagaataattggaatagattatattatccttctatttaaattattctgaataaagaggaggagagtgagta

SEQ ID NO: 190 - DNA, 380 bp, Bacillus licheniformis
  misc_feature (1)..(380): B. licheniformis ATCC14580; 5-prime region to aprE gene comprising promoter aprE (database Acc. CP000002)
atcttacacccgtttctgtatgcgatatattgcatattttaatagatgatcgactaggccgcaacctccttcggcaaaaaatgatctcataaaataaatgaatagtattttcataaaatgaatcagacgaagcaatctcctgtcattcacggaccccgggacctctttccctgccaggttgaagcggtctattcatactttcgaaccgaatatttttctaaaacagttattaataaccaataaatttaaattggccgttcaaaaaaatgggtctaccatataattcattttttttctataataaattaacagaataattagaatagagtatattattcttctatttcaattattctgaataaaacggaggagagtgagta

SEQ ID NO: 191 - DNA, 226 bp, Bacillus licheniformis
  misc_feature (1)..(226): B. licheniformis ATCC14580; Promoter aprE trunc. DSM13 (database Acc. CP000002)
cccgggacctctttccctgccaggttgaagcggtctattcatactttcgaaccgaatatttttctaaaacagttattaataaccaataaatttaaattggccgttcaaaaaaatgggtctaccatataattcattttttttctataataaattaacagaataattagaatagagtatattattcttctatttcaattattctgaataaaacggaggagagtgagta

SEQ ID NO: 192 - DNA, 374 bp, Bacillus licheniformis
  misc_feature (1)..(374): B. licheniformis ATCC14580; Promoter aprE fl- DSM13 (database Acc. CP000002)
cacccgtttctgtatgcgatatattgcatattttaatagatgatcgactaggccgcaacctccttcggcaaaaaatgatctcataaaataaatgaatagtattttcataaaatgaatcagacgaagcaatctcctgtcattcacggaccccgggacctctttccctgccaggttgaagcggtctattcatactttcgaaccgaatatttttctaaaacagttattaataaccaataaatttaaattggccgttcaaaaaaatgggtctaccatataattcattttttttctataataaattaacagaataattagaatagagtatattattcttctatttcaattattctgaataaaacggaggagagtgagta

SEQ ID NO: 193 - DNA, 434 bp, Bacillus subtilis
  misc_feature (1)..(434): B. subtilis subsp. 168; 5-prime region to aprE gene comprising promoter aprE (database Acc. AL009126)
cttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaaaga

SEQ ID NO: 194 - DNA, 222 bp, Bacillus subtilis
  misc_feature (1)..(222): B. subtilis subsp. 168; Promoter aprE -222 (database Acc. AL009126)
gcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaaaga

SEQ ID NO: 195 - DNA, 175 bp, Bacillus subtilis
  misc_feature (1)..(175): B. subtilis subsp. 168; Promoter bpr -175 (database Acc. AL009126)
tctcttcacactttccttcttataaagtctttttccctattgcttccttcgcttagtaacaaaacagataattagacccatttatttttgtgacatttttatcattttcatatatatggaaattgaatgacatgaaacgacaatatctgtaattcagattgtctacagttaatat

SEQ ID NO: 196 - DNA, 259 bp, Bacillus subtilis
  misc_feature (1)..(259): B. subtilis subsp. 168; 5-prime region to bpr gene comprising promoter bpr (database Acc. AL009126)
tctcttcacactttccttcttataaagtctttttccctattgcttccttcgcttagtaacaaaacagataattagacccatttatttttgtgacatttttatcattttcatatatatggaaattgaatgacatgaaacgacaatatctgtaattcagattgtctacagttaatatacagcgatgttctgacaaaccattcattattaaaaggagggacgacactttttttaaaaagcatgttgaaaaagggggatgaaa

SEQ ID NO: 197 - DNA, 191 bp, Bacillus licheniformis
  misc_feature (1)..(191): B. licheniformisATCC14580; 5-prime region to bpr gene comprising promoter bpr (database Acc. CP000002)
taaaaaaggacaaaacctttaagcggttttgtcctttttttatctaattttaaacaaggccttgacaaataccgacaaaataattaaccagtttttactatatttaagttacatcaaagtcagacaaggcgtccacaaggagggggggacaggaaaattcagctaatttcataaaaaagggggatgttcat

SEQ ID NO: 198 - DNA, 344 bp, Bacillus subtilis
  misc_feature (1)..(344): B. subtilis subsp. 168; 5-prime region to sacB gene comprising promoter sacB (database Acc. AL009126)
taaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacg

SEQ ID NO: 199 - DNA, 350 bp, Bacillus licheniformis
  misc_feature (1)..(350): B. licheniformis ATCC14580; 5-prime region to sacB gene comprising promoter sacB (database Acc. CP000002)
acgattcccgcttatacagactatagattcatataaaaaaggtgtctttccgcttaaaatcggtgtcatttgcataaaaatgtataggaaaagaggaacttagaccagttgagtcccagagatgagtatattagaatgatggtaattcaatatcgtcgggattgttactgtctaagcaggcaagacctaaaatgtgtgaagggcgaaatctatcttttgcctatatgaacttgcacattttaggtcttttttctgttcttcaggacaatgccgcagttaggagggatgattgggaacgttcatgttgtcagaagcaatctattacatattgaaaaagggaggaaatattg

SEQ ID NO: 200 - DNA, 700 bp, Bacillus subtilis
  misc_feature (1)..(700): B. subtilis subsp. 168; 5-prime region to ywsC gene comprising promoter ywsC (database Acc. AL009126)
aaatctaattgtaaattgtatatataattgaaaaatttaaggttttcactctttttttgacaaagtcttcacaaatatggatgaagatttcttgaacaaatattaagttttattaaaattgtttcaaagtcatgcgtttagacctgtaaatggggagaaacagaaaagactatcatcccagaaaatgtttaattatgttagagaattcggactcgtatgtttagcgttttcatctcgaacctccttagtcattttggaatgggcctcatattgcttattccatgcagaaaggatgacattatgaccttgtccttaagaaacaggataaaattattttgtgttcataagaaccggaaacacgcatgatagcaggaaatgcttgataacaaaggctgaaaaaaagattgatttgaaatgcatattaaatacaaacagcagggtgtgactatacgtccagaaggatatcaggagaaaaatgaaagcgcattctctttgatttttttaaaaagtgataaggtataagtcctgctgttttccaaaaatagaaaacagtttgtaggtataaaatctctttcaaaagagaagtttggcttagtcgattagggaagattatgttacataatgccgattgagaattcatagtgattctatatactgatgaatgaatttacaacaatatagaaggagatgtcgaaaagca

SEQ ID NO: 201 - DNA, 700 bp, Bacillus licheniformis
  misc_feature (1)..(700): B. licheniformis ATCC14580; 5-prime region to ywsC gene comprising promoter ywsC (database Acc. CP000002)
tttagaccgggctgggatatcgagacatatataggggcgtttaatggcgaatacagtaacaatgagaatagtaagaaaaattaaaaatgttaaagtttgatgaattatcattgaaaaaaattaatggctttttaaatcctaggattttaacctaaaatctgaagaaataaggtggatcgaacgactcacaaaatatttggatttgtcaatgaatcccgctttatgctaaaagagattttcattttttgatagatggtctgattgtcataggacggatttgttttgaagagggaacattggtgactttttaacctgttcgaaaagagcgaaaatactaaaagaaaagagagatcccggctgacagcccatttaaaggggattgcggacgggggaaaaaagagatcctgaatccatccttcaacctttcatctgaaatagggagaaaagtacaaaaatcataatgtcgaattttgaaagcgcatacttaaaacgctgacaaaaatctgataggaattaagaactttcgatttccaaaaatatcaataaaaagataggcattaatgactcgggcgaggtgatctttgtcacggaaaatttcgtcgtcttctgttacataatgccgattgtgatttcatagtgaacctatatactgatgaatgaatttacatatcagattccaagaaggagatgtagacaaaca

SEQ ID NO: 202 - DNA, 382 bp, Bacillus pumilus
  misc_feature (1)..(382): 5-prime region to aprE1 gene comprising promoter aprE1
ctgttatataaacaggttcttttaaatgacaaaaacaatgataaaataatatttttttatatcgaaattcgaaatagctgctagacgtttctacctattttaaggcttttcgggtatcgaatatttctccgataatggatcataagaaaaatagcatacttcctttttaatagataatcgctgaaacagtagaataaacatattttaccactatttccaagtgacttaattccccaattttcgctaggactttcacaaaaattcaggtctactcttatttgcctacttcccttaaactgaatatacagaataatcaaacgtctcattcttatagactacggatgattattctgaaataagaaaaaagggatgtggattgtgc

SEQ ID NO: 203 - DNA, 303 bp, Bacillus pumilus
  misc_feature (1)..(303): 5-prime region to aprE2 gene comprising promoter aprE2
cgagaacatcttgaaaggcagcacagccgaaacccggaattgtcgagagcgaacatgccgcactttagtacacatgaaagaaagacaaaaacaaaacaagaagccttgcagcacatgataggaaaacataaaagaaggaatgcttatgatcactatcaagaggatcataagcatttttattttgcttttttgtgacaactagcttaaataaatggaaaaatttaaaaaataggacaaacgatcgtttctttttgtctaaaaatgtagttaaatgacaaaaagatgaaagggagagtgggtatt

SEQ ID NO: 204 - DNA, 246 bp, Bacillus subtilis
  misc_feature (1)..(246): remB
ttgtatattcatttaggtgatgactttgtggtttcaacacgagatattgtcggcatttttgactttaaagccaacatgtcgcctattgttgaagaatttctgaaaaaacagaaacacaaggtggtgccttccgtaaacggcacgcccaaatctatcgtagtcacggttcagaatatatattactctcccttatcttccagcacattaaaaaaacgtgcgcaatttatgtttgaaatagattcttag

SEQ ID NO: 205 - protein, 81 aa, Bacillus subtilis
  MISC_FEATURE (1)..(81): remB
MYIHLGDDFVVSTRDIVGIFDFKANMSPIVEEFLKKQKHKVVPSVNGTPKSIVVTVQNIYYSPLSSSTLKKRAQFMFEIDS

SEQ ID NO: 206 - DNA, 243 bp, Bacillus licheniformis
  misc_feature (1)..(243): remB
ttgtatatccacttaggtgacgattttgtcgtctcaacgcgtgaaattgtggctatttttgattacaaggcaaagacatcgccgattgttgaggagtttttaagcaagcaaaaacagcggattgtctcttctaacagcacgccgaagtcaattgttgtcacattacaatcgatttatttttctcctttagcctcaggcacgttgaaaaaacgggcgcaatccaagccggaaatcgattcataa

SEQ ID NO: 207 - protein, 80 aa, Bacillus licheniformis
  MISC_FEATURE (1)..(80): remB
MYIHLGDDFVVSTREIVAIFDYKAKTSPIVEEFLSKQKQRIVSSNSTPKSIVVTLQSIYFSPLASGTLKKRAQSKPEIDS

SEQ ID NO: 208 - DNA, 59 bp, Artificial Sequence
  Sequence in Figure 2
catacttttcggtgaaaaatgagccgaaagcagacacactattagtaacagatcaaata

SEQ ID NO: 209 - DNA, 59 bp, Artificial Sequence
  Sequence in Figure 2
catacttttcggtgaaaaatgagccgaaagcagatacactattagtaacagatcaaata

SEQ ID NO: 210 - DNA, 51 bp, Artificial Sequence
  Sequence in Figure 2
tcggtgaaaaagaaaccaaatccctttaaacttgtattaacagatcaaata

SEQ ID NO: 211 - DNA, 59 bp, Artificial Sequence
  Sequence in Figure 2
catacttttcggtgaaaaaatactcaattacatttacactgttagtaacagatcaaata

SEQ ID NO: 212 - DNA, 59 bp, Artificial Sequence
  Sequence in Figure 2
catacttttcggtgaaaaatcccgcaaaaacgtttacactattagtaacagatcaaata

SEQ ID NO: 213 - DNA, 59 bp, Artificial Sequence; identical to SEQ ID NO: 212
  Sequence in Figure 2

SEQ ID NO: 214 - DNA, 381 bp, Bacillus licheniformis
  misc_feature (1)..(381): CDS of forD
atgaaaaatcatttgtatgagaaaaaaaagaggaaacctttgactcggacaattaaagcgacgctcgccgtgttgacaatgtccatcgctttggtgggaggcgctacggtgccttcatttgcatgggtgaatccgggttatcactaccagtacccatcggaaggtggtacatggaggtatggattcgtaaacgccgggctccgttcagagtacaaccacccgacaaaggtccacggctcgacagtgcaaaagctcatcgatggaaaagtggataaaacgaatagaagtattgatacggctgcgggccgctactctaatgcctatgtcggagccataaactcacctggtcttaagggtcgttactactatcgcaccaactaa

SEQ ID NO: 215 - protein, 126 aa, Bacillus licheniformis
  MISC_FEATURE (1)..(126): Protein of ForD (Formosin)
MKNHLYEKKKRKPLTRTIKATLAVLTMSIALVGGATVPSFAWVNPGYHYQYPSEGGTWRYGFVNAGLRSEYNHPTKVHGSTVQKLIDGKVDKTNRSIDTAAGRYSNAYVGAINSPGLKGRYYYRTN

SEQ ID NO: 216 - DNA, 1055 bp, Artificial Sequence
  Homology regions of Fusion of 5-prime and 3-prime regions of forD gene with flanking BsaI sites
ggtctcgacccggatcccattcggagtatttctgaatgatagagccacacggtccacgttctcactggctaaccggatcaaatgatcttcaggagtcagcataatacatccagttcaggtagataagatttgaatttggtgacttgcttttgttcttcttctttcattttctgactaatccaaactggaaaaagcaggtcttttaacagattaggaggtttctgacatgcaccattcggtcactaaccgaatgcagtaaaggacactgtggtgcttgccagccattagggtattgaggaggtgatcaaaatgctaggtgacagtatttcgtcgaagtggacaagtcgtgaccaaatgacctcggatcgagggttggtcatggaggaaaaaattgatgtctggtgacaaagaggagtcatgatcatggcaccgccaacgagggaaaaaactcttcccgcatcgacacggtatgtgggcggtgacaaactaacttatagagtaaatttattagtcgaatgaaagctcctccatgaaatataatcaaagggaaaacggttgctgtcaacggggctagcatggcaagacccagaaaagttctgggagatcccgctttgcataagcgtattatagtggatgacgcgggctttgttgtttacacttcttgcacctgctgacggcaatcatccctatctatgaaatcgagatttcagcaggccgttattttcgagagagttaaatctatattcattgtttttattttggtaaggacataccggattttaggtttggattaccggtcgagttagcttgtcttttcgcccactaccgtgtcgatgcgggagcaatttaccagaagcacttaccgattgatagttttttattccggtgattgcaaagtttcataaactctgagaattcaataggggtaataccccgctttgaggggcgcggcattttatgcgccccgagtatttattcttaaaatttttaaattaatgtatctatataaaaaggagatgctttcggtgtactgccaaaccatggctcaggagacc

SEQ ID NO: 217 - DNA, 1022 bp, Artificial Sequence
  Homology region of the fusion of 5-prime and 3-prime regions of amyB gene with flanking BsaI sites
ggtctcgacccacaaggctgtcaaacgaaaaagcgtatcaggagattaacgacacgcaagaaatgatcgaaaaaatcagcggacacctgcctgtacacttgcgtcctccatacggcgggatcaatgattccgtccgctcgctttccaatctgaaggtttcattgtgggatgttgatccggaagattggaagtacaaaaataagcaaaagattgtcaatcatgtcatgagccatgcgggagacggaaaaatcgtcttaatgcacgatatttatgcaacgtccgcagatgctgctgaagagattattaaaaagctgaaagcaaaaggctatcaattggtaactgtatctcagcttgaagaagtgaagaagcagagaggctattgaataaatgagtagaaagcgccatatcggcgcttttcttttggaagaaaatatagggaaaatggtatttgttaaaaattcggaatatttatacaatatcatatgtttcacattgaaaggggaggagaatctagaagagcagagaggacggatttcctgaaggaaatccgtttttttattttgcccgtcttataaatttctttgattacattttataattaattttaacaaagtgtcatcagccctcaggaaggacttgctgacagtttgaatcgcataggtaaggcggggatgaaatggcaacgttatctgatgtagcaaagaaagcaaatgtgtcgaaaatgacggtatcgcgggtgatcaatcatcctgagactgtgacggatgaattgaaaaagcttgttcattccgcaatgaaggagctcaattatataccgaactatgcagcaagagcgctcgttcaaaacagaacacaggtcgtcaagctgctcatactggaagaaatggatacaacagaaccttattatatgaatctgttaacgggaatcagccgcgagctggaccgtcatcattatgctttgcagcttgtcacaaggaaatctctcaatatcggccagtgcgacggcattatctcaggagacc

SEQ ID NO: 218 - DNA, 794 bp, Artificial Sequence; identical to SEQ ID NO: 55
  Homology regions of gene synthesis HomA + HomB remA R18W, P29S